SEQUENCE LISTING (158 sequences)

SEQ ID NO: 1 - protein, 115 aa, synthetic construct
DVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSS

SEQ ID NO: 2 - protein, 325 aa, synthetic construct
DVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 111 aa, synthetic construct
DIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIK

SEQ ID NO: 4 - protein, 218 aa, synthetic construct
DIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 115 aa, synthetic construct
QVQLQESGPGLVKPSQTLSLTCTVSGGSISSDSAWNWIRQHPGKGLEWIGYISYSGSTSYNPSLKSRVTISRDTSKNQFSLKLSSVTAADTAVYYCVRGLRFAYWGQGTLVTVSS

SEQ ID NO: 6 - protein, 445 aa, synthetic construct
QVQLQESGPGLVKPSQTLSLTCTVSGGSISSDSAWNWIRQHPGKGLEWIGYISYSGSTSYNPSLKSRVTISRDTSKNQFSLKLSSVTAADTAVYYCVRGLRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 111 aa, synthetic construct
EIVLTQSPATLSLSPGERATLSCRASESVDSYVNSFLHWYQQKPGQAPRLLIYRASNLQSGIPARFSGSGSRTDFTLTISSLEPEDVAVYYCQQSNEDPYTFGQGTKLEIK

SEQ ID NO: 8 - protein, 218 aa, synthetic construct
EIVLTQSPATLSLSPGERATLSCRASESVDSYVNSFLHWYQQKPGQAPRLLIYRASNLQSGIPARFSGSGSRTDFTLTISSLEPEDVAVYYCQQSNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 115 aa, synthetic construct
QVQLQESGPGLVKPSETLSLTCTVSGGSISSDSAWNWIRQPPGKGLEWIGYISYSGSTSYNPSLKSRVTISRDTSKNQFSLKLSSVTAADTAVYYCVRGLRFAYWGQGTLVTVSS

SEQ ID NO: 10 - protein, 445 aa, synthetic construct
QVQLQESGPGLVKPSETLSLTCTVSGGSISSDSAWNWIRQPPGKGLEWIGYISYSGSTSYNPSLKSRVTISRDTSKNQFSLKLSSVTAADTAVYYCVRGLRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 111 aa, synthetic construct; identical to SEQ ID NO: 7

SEQ ID NO: 12 - protein, 218 aa, synthetic construct; identical to SEQ ID NO: 8

SEQ ID NO: 13 - protein, 115 aa, synthetic construct
QLQLQESGPGLVKPSETLSLTCTVSGGSISSDSAWNWIRQPPGKGLEWIGYISYSGSTSYNPSLKSRVTISRDTSKNQFSLKLSSVTAADTAVYYCVRGLRFAYWGQGTLVTVSS

SEQ ID NO: 14 - protein, 445 aa, synthetic construct
QLQLQESGPGLVKPSETLSLTCTVSGGSISSDSAWNWIRQPPGKGLEWIGYISYSGSTSYNPSLKSRVTISRDTSKNQFSLKLSSVTAADTAVYYCVRGLRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 111 aa, synthetic construct; identical to SEQ ID NO: 7

SEQ ID NO: 16 - protein, 218 aa, synthetic construct; identical to SEQ ID NO: 8

SEQ ID NO: 17 - protein, 115 aa, synthetic construct
QVQLQESGPGLVKPSETLSLTCTVSGYSITSDSAWNWIRQPPGKGLEWIGYISYSGSTSYNPSLKSRVTISRDTSKNQFSLKLSSVTAADTAVYYCVRGLRFAYWGQGTLVTVSS

SEQ ID NO: 18 - protein, 445 aa, synthetic construct
QVQLQESGPGLVKPSETLSLTCTVSGYSITSDSAWNWIRQPPGKGLEWIGYISYSGSTSYNPSLKSRVTISRDTSKNQFSLKLSSVTAADTAVYYCVRGLRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 111 aa, synthetic construct; identical to SEQ ID NO: 7

SEQ ID NO: 20 - protein, 218 aa, synthetic construct; identical to SEQ ID NO: 8

SEQ ID NO: 21 - protein, 115 aa, synthetic construct
QVQLQESGPGLVKPSQTLSLTCTVSGYSITSDSAWNWIRQPPGKGLEWIGYISYSGSTSYNPSLKSRVTISRDTSKNQFSLKLSSVTAADTAVYYCVRGLRFAYWGQGTLVTVSS

SEQ ID NO: 22 - protein, 445 aa, synthetic construct
QVQLQESGPGLVKPSQTLSLTCTVSGYSITSDSAWNWIRQPPGKGLEWIGYISYSGSTSYNPSLKSRVTISRDTSKNQFSLKLSSVTAADTAVYYCVRGLRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 111 aa, synthetic construct; identical to SEQ ID NO: 7

SEQ ID NO: 24 - protein, 218 aa, synthetic construct; identical to SEQ ID NO: 8

SEQ ID NO: 25 - protein, 115 aa, synthetic construct; identical to SEQ ID NO: 5

SEQ ID NO: 26 - protein, 445 aa, synthetic construct; identical to SEQ ID NO: 6

SEQ ID NO: 27 - protein, 111 aa, synthetic construct
DIVLTQSPDSLAVSLGERATINCRASESVDSYVNSFLHWYQQKPGQPPKLLIYRASNLQSGVPDRFSGSGSRTDFTLTISSLQAEDVAVYYCQQSNEDPYTFGQGTKLEIK

SEQ ID NO: 28 - protein, 218 aa, synthetic construct
DIVLTQSPDSLAVSLGERATINCRASESVDSYVNSFLHWYQQKPGQPPKLLIYRASNLQSGVPDRFSGSGSRTDFTLTISSLQAEDVAVYYCQQSNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 115 aa, synthetic construct; identical to SEQ ID NO: 9

SEQ ID NO: 30 - protein, 445 aa, synthetic construct; identical to SEQ ID NO: 10

SEQ ID NO: 31 - protein, 111 aa, synthetic construct; identical to SEQ ID NO: 27

SEQ ID NO: 32 - protein, 218 aa, synthetic construct; identical to SEQ ID NO: 28

SEQ ID NO: 33 - protein, 115 aa, synthetic construct; identical to SEQ ID NO: 13

SEQ ID NO: 34 - protein, 445 aa, synthetic construct; identical to SEQ ID NO: 14

SEQ ID NO: 35 - protein, 111 aa, synthetic construct; identical to SEQ ID NO: 27

SEQ ID NO: 36 - protein, 218 aa, synthetic construct; identical to SEQ ID NO: 28

SEQ ID NO: 37 - protein, 115 aa, synthetic construct; identical to SEQ ID NO: 17

SEQ ID NO: 38 - protein, 445 aa, synthetic construct; identical to SEQ ID NO: 18

SEQ ID NO: 39 - protein, 111 aa, synthetic construct; identical to SEQ ID NO: 27

SEQ ID NO: 40 - protein, 218 aa, synthetic construct; identical to SEQ ID NO: 28

SEQ ID NO: 41 - protein, 115 aa, synthetic construct; identical to SEQ ID NO: 21

SEQ ID NO: 42 - protein, 445 aa, synthetic construct; identical to SEQ ID NO: 22

SEQ ID NO: 43 - protein, 111 aa, synthetic construct; identical to SEQ ID NO: 27

SEQ ID NO: 44 - protein, 218 aa, synthetic construct; identical to SEQ ID NO: 28

SEQ ID NO: 45 - protein, 115 aa, synthetic construct; identical to SEQ ID NO: 5

SEQ ID NO: 46 - protein, 445 aa, synthetic construct; identical to SEQ ID NO: 6

SEQ ID NO: 47 - protein, 111 aa, synthetic construct
DIQLTQSPSSLSASVGDRVTITCRASESVDSYVNSFLHWYQQKPGKAPKLLIYRASNLQSGVPSRFSGSGSRTDFTLTISSLQPEDVATYYCQQSNEDPYTFGQGTKLEIK

SEQ ID NO: 48 - protein, 218 aa, synthetic construct
DIQLTQSPSSLSASVGDRVTITCRASESVDSYVNSFLHWYQQKPGKAPKLLIYRASNLQSGVPSRFSGSGSRTDFTLTISSLQPEDVATYYCQQSNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 115 aa, synthetic construct; identical to SEQ ID NO: 9

SEQ ID NO: 50 - protein, 445 aa, synthetic construct; identical to SEQ ID NO: 10

SEQ ID NO: 51 - protein, 111 aa, synthetic construct; identical to SEQ ID NO: 47

SEQ ID NO: 52 - protein, 218 aa, synthetic construct; identical to SEQ ID NO: 48

SEQ ID NO: 53 - protein, 115 aa, synthetic construct; identical to SEQ ID NO: 13

SEQ ID NO: 54 - protein, 445 aa, synthetic construct; identical to SEQ ID NO: 14

SEQ ID NO: 55 - protein, 111 aa, synthetic construct; identical to SEQ ID NO: 47

SEQ ID NO: 56 - protein, 218 aa, synthetic construct; identical to SEQ ID NO: 48

SEQ ID NO: 57 - protein, 115 aa, synthetic construct; identical to SEQ ID NO: 17

SEQ ID NO: 58 - protein, 445 aa, synthetic construct; identical to SEQ ID NO: 18

SEQ ID NO: 59 - protein, 111 aa, synthetic construct; identical to SEQ ID NO: 47

SEQ ID NO: 60 - protein, 218 aa, synthetic construct; identical to SEQ ID NO: 48

SEQ ID NO: 61 - protein, 115 aa, synthetic construct; identical to SEQ ID NO: 21

SEQ ID NO: 62 - protein, 445 aa, synthetic construct; identical to SEQ ID NO: 22

SEQ ID NO: 63 - protein, 111 aa, synthetic construct; identical to SEQ ID NO: 47

SEQ ID NO: 64 - protein, 218 aa, synthetic construct; identical to SEQ ID NO: 48

SEQ ID NO: 65 - DNA, 345 bp, synthetic construct
  source 1..345: mol_type = other DNA
gatgtgcagctgcagcagagcggacctggattggtgaagccttcccagtccctgagcctgacctgtaccgtgaccggatattctattacttccgactccgcttggaactggattaggcagttccccggcaacaggctggaatggatgggatatatcagctactccggctccacctcctacaacccctccttgaagtcccggatctccatcacccgcgatacctccaagaaccagttcttcctgcagctcaactctgtgaccaccgaggacaccgccacctactactgcgtgaggggactgcgattcgcctactggggacagggtaccctcgttaccgtgtctagc

SEQ ID NO: 66 - DNA, 1335 bp, synthetic construct
  source 1..1335: mol_type = other DNA
gatgtgcagctgcagcagagcggacctggattggtgaagccttcccagtccctgagcctgacctgtaccgtgaccggatattctattacttccgactccgcttggaactggattaggcagttccccggcaacaggctggaatggatgggatatatcagctactccggctccacctcctacaacccctccttgaagtcccggatctccatcacccgcgatacctccaagaaccagttcttcctgcagctcaactctgtgaccaccgaggacaccgccacctactactgcgtgaggggactgcgattcgcctactggggacagggtaccctcgttaccgtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 67 - DNA, 333 bp, synthetic construct
  source 1..333: mol_type = other DNA
gatatcgtgctgacccagtcccccgcttccttggctgtttccctgggacagagggccaccatctcctgtagggcttccgagtccgtggactcctacgtgaacagctttttgcactggtaccagcagaagcccggccagcctcctaagttgctcatctacagggcttccaacctgcagtccggcattccagcccgtttttccgggtccggttccagaaccgacttcaccctcaccatcaaccccgtcgaggcagacgacgtcgctacttactactgccagcagtccaacgaagatccttataccttcggaggcggcaccaagctcgaaatcaaa

SEQ ID NO: 68 - DNA, 654 bp, synthetic construct
  source 1..654: mol_type = other DNA
gatatcgtgctgacccagtcccccgcttccttggctgtttccctgggacagagggccaccatctcctgtagggcttccgagtccgtggactcctacgtgaacagctttttgcactggtaccagcagaagcccggccagcctcctaagttgctcatctacagggcttccaacctgcagtccggcattccagcccgtttttccgggtccggttccagaaccgacttcaccctcaccatcaaccccgtcgaggcagacgacgtcgctacttactactgccagcagtccaacgaagatccttataccttcggaggcggcaccaagctcgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 69 - DNA, 345 bp, synthetic construct
  source 1..345: mol_type = other DNA
caggttcagctccaggagtcaggcccaggactcgttaaaccctcccagaccctctccctcacctgcacagtctccggtggaagcatctcctccgactccgcatggaactggatcagacagcaccccggcaaaggcctcgaatggattggctatatctcctactccggctccacttcctacaatccctccctcaagtccagagtgaccatctcccgcgacacctcaaagaaccagttctccctcaaactgagcagcgtgaccgccgctgacaccgctgtttactactgcgttcgtggcctccgcttcgcctactggggacagggaacactcgtgaccgtgtcctcc

SEQ ID NO: 70 - DNA, 1335 bp, synthetic construct
  source 1..1335: mol_type = other DNA
caggttcagctccaggagtcaggcccaggactcgttaaaccctcccagaccctctccctcacctgcacagtctccggtggaagcatctcctccgactccgcatggaactggatcagacagcaccccggcaaaggcctcgaatggattggctatatctcctactccggctccacttcctacaatccctccctcaagtccagagtgaccatctcccgcgacacctcaaagaaccagttctccctcaaactgagcagcgtgaccgccgctgacaccgctgtttactactgcgttcgtggcctccgcttcgcctactggggacagggaacactcgtgaccgtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 71 - DNA, 333 bp, synthetic construct
  source 1..333: mol_type = other DNA
gaaatcgtgctgacccagtcccccgctaccttgagtctgtcccctggagagagagccaccctgtcttgtagggctagtgagagcgtggactcctacgtgaactcctttctgcactggtaccagcagaagccaggccaggctcctcgattgttgatctacagagccagcaacttgcagtccgggattcctgccaggttttccggctccggttccagaaccgactttaccctcaccatctcctccctggagcccgaagatgtcgccgtttactactgccagcagtccaacgaggacccctacaccttcggtcagggcactaagctcgaaatcaag

SEQ ID NO: 72 - DNA, 654 bp, synthetic construct
  source 1..654: mol_type = other DNA
gaaatcgtgctgacccagtcccccgctaccttgagtctgtcccctggagagagagccaccctgtcttgtagggctagtgagagcgtggactcctacgtgaactcctttctgcactggtaccagcagaagccaggccaggctcctcgattgttgatctacagagccagcaacttgcagtccgggattcctgccaggttttccggctccggttccagaaccgactttaccctcaccatctcctccctggagcccgaagatgtcgccgtttactactgccagcagtccaacgaggacccctacaccttcggtcagggcactaagctcgaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 73 - DNA, 345 bp, synthetic construct
  source 1..345: mol_type = other DNA
caggttcagctccaggagtcaggcccaggactcgttaaaccctccgagacactctccctgacatgcacagtctccggcggaagcatctcctccgactcagcttggaactggatcaggcagccccccggaaagggtttggagtggattggctatatctcctactccggctccacctcctacaacccaagcctgaaaagccgagtgaccatctctagggacacctccaagaaccagttctccctcaaactgagcagcgtgaccgccgctgacaccgctgtttactactgcgtccgcggcctgaggttcgcttactggggacagggaaccctcgtcaccgtgtctagc

SEQ ID NO: 74 - DNA, 1335 bp, synthetic construct
  source 1..1335: mol_type = other DNA
caggttcagctccaggagtcaggcccaggactcgttaaaccctccgagacactctccctgacatgcacagtctccggcggaagcatctcctccgactcagcttggaactggatcaggcagccccccggaaagggtttggagtggattggctatatctcctactccggctccacctcctacaacccaagcctgaaaagccgagtgaccatctctagggacacctccaagaaccagttctccctcaaactgagcagcgtgaccgccgctgacaccgctgtttactactgcgtccgcggcctgaggttcgcttactggggacagggaaccctcgtcaccgtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 75 - DNA, 333 bp, synthetic construct; identical to SEQ ID NO: 71
  source 1..333: mol_type = other DNA

SEQ ID NO: 76 - DNA, 654 bp, synthetic construct; identical to SEQ ID NO: 72
  source 1..654: mol_type = other DNA

SEQ ID NO: 77 - DNA, 345 bp, synthetic construct
  source 1..345: mol_type = other DNA
cagttgcagctccaggagtccggtccaggactcgttaaaccctccgagacactctccctcacctgcacagtctccggcggaagtatctcctccgactccgcttggaactggatcaggcagcctcccggtaaggggttggagtggatcggatatatctcctacagcggctccacctcctacaatcccagtctgaagtccagagtgaccatctcccgcgacacctccaagaaccagttctccctcaaactgtccagcgtgaccgccgcagacaccgctgtttactactgcgtgcgcggcctgagatttgcctactggggacagggcaccctcgtgacagtgtctagc

SEQ ID NO: 78 - DNA, 1335 bp, synthetic construct
  source 1..1335: mol_type = other DNA
cagttgcagctccaggagtccggtccaggactcgttaaaccctccgagacactctccctcacctgcacagtctccggcggaagtatctcctccgactccgcttggaactggatcaggcagcctcccggtaaggggttggagtggatcggatatatctcctacagcggctccacctcctacaatcccagtctgaagtccagagtgaccatctcccgcgacacctccaagaaccagttctccctcaaactgtccagcgtgaccgccgcagacaccgctgtttactactgcgtgcgcggcctgagatttgcctactggggacagggcaccctcgtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 79 - DNA, 333 bp, synthetic construct; identical to SEQ ID NO: 71
  source 1..333: mol_type = other DNA

SEQ ID NO: 80 - DNA, 654 bp, synthetic construct; identical to SEQ ID NO: 72
  source 1..654: mol_type = other DNA

SEQ ID NO: 81 - DNA, 345 bp, synthetic construct
  source 1..345: mol_type = other DNA
caggttcagctccaggagtcaggcccaggactcgttaaaccctccgagacactctccctgacatgcacagtctccggctactccatcacatcagactccgcctggaactggatcagacagcccccaggaaaggggctcgaatggattggctatatctcctactccggctctacctcctacaacccctccctgaagtcccgcgtcaccatctctcgcgacacctctaagaaccagttctccctcaaactgtcctccgtgaccgccgctgacaccgctgtttactactgcgtgcgcggcctgagatttgcctactggggacagggcaccctcgtgacagtgtctagc

SEQ ID NO: 82 - DNA, 1335 bp, synthetic construct
  source 1..1335: mol_type = other DNA
caggttcagctccaggagtcaggcccaggactcgttaaaccctccgagacactctccctgacatgcacagtctccggctactccatcacatcagactccgcctggaactggatcagacagcccccaggaaaggggctcgaatggattggctatatctcctactccggctctacctcctacaacccctccctgaagtcccgcgtcaccatctctcgcgacacctctaagaaccagttctccctcaaactgtcctccgtgaccgccgctgacaccgctgtttactactgcgtgcgcggcctgagatttgcctactggggacagggcaccctcgtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 83 - DNA, 333 bp, synthetic construct; identical to SEQ ID NO: 71
  source 1..333: mol_type = other DNA

SEQ ID NO: 84 - DNA, 654 bp, synthetic construct; identical to SEQ ID NO: 72
  source 1..654: mol_type = other DNA

SEQ ID NO: 85 - DNA, 345 bp, synthetic construct
  source 1..345: mol_type = other DNA
caggttcagctccaggagtcaggcccaggactcgttaaaccctcccagaccctctccctcacctgcacagtctccggttactccatcacctccgacagcgcatggaactggatcagacagccccccggaaaaggcctcgaatggattggctatatctcctactccggctccacctcctacaacccctccttgaagtcccgcgtgaccatcagccgggacacatcaaagaaccagttctccctcaagctgtctagcgtgactgccgccgacaccgctgtttactactgcgtgcggggcctgagatttgcctactggggacagggcaccctggtgacagtgtctagc

SEQ ID NO: 86 - DNA, 1335 bp, synthetic construct
  source 1..1335: mol_type = other DNA
caggttcagctccaggagtcaggcccaggactcgttaaaccctcccagaccctctccctcacctgcacagtctccggttactccatcacctccgacagcgcatggaactggatcagacagccccccggaaaaggcctcgaatggattggctatatctcctactccggctccacctcctacaacccctccttgaagtcccgcgtgaccatcagccgggacacatcaaagaaccagttctccctcaagctgtctagcgtgactgccgccgacaccgctgtttactactgcgtgcggggcctgagatttgcctactggggacagggcaccctggtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - DNA, 333 bp, synthetic construct; identical to SEQ ID NO: 71
  source 1..333: mol_type = other DNA

SEQ ID NO: 88 - DNA, 654 bp, synthetic construct; identical to SEQ ID NO: 72
  source 1..654: mol_type = other DNA

SEQ ID NO: 89 - DNA, 345 bp, synthetic construct; identical to SEQ ID NO: 69
  source 1..345: mol_type = other DNA

SEQ ID NO: 90 - DNA, 1335 bp, synthetic construct; identical to SEQ ID NO: 70
  source 1..1335: mol_type = other DNA

SEQ ID NO: 91 - DNA, 333 bp, synthetic construct
  source 1..333: mol_type = other DNA
gatatcgtgctgacccagtcccccgactccttggctgtttccctgggagagagggccaccatcaactgtagggcctccgagtccgtggactcctacgttaacagctttctgcattggtaccagcagaagcccggccagcctcctaagttgctcatttacagggcttccaacctccagtccggcgtgcctgatcgtttctctggctccggttcccgcaccgatttcaccttgaccatctcctccctgcaggctgaggacgtcgctgtttactactgccagcagtccaacgaagatccttatacctttggacagggcaccaaactcgaaattaag

SEQ ID NO: 92 - DNA, 654 bp, synthetic construct
  source 1..654: mol_type = other DNA
gatatcgtgctgacccagtcccccgactccttggctgtttccctgggagagagggccaccatcaactgtagggcctccgagtccgtggactcctacgttaacagctttctgcattggtaccagcagaagcccggccagcctcctaagttgctcatttacagggcttccaacctccagtccggcgtgcctgatcgtttctctggctccggttcccgcaccgatttcaccttgaccatctcctccctgcaggctgaggacgtcgctgtttactactgccagcagtccaacgaagatccttatacctttggacagggcaccaaactcgaaattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 93 - DNA, 345 bp, synthetic construct; identical to SEQ ID NO: 73
  source 1..345: mol_type = other DNA

SEQ ID NO: 94 - DNA, 1335 bp, synthetic construct; identical to SEQ ID NO: 74
  source 1..1335: mol_type = other DNA

SEQ ID NO: 95 - DNA, 333 bp, synthetic construct; identical to SEQ ID NO: 91
  source 1..333: mol_type = other DNA

SEQ ID NO: 96 - DNA, 654 bp, synthetic construct; identical to SEQ ID NO: 92
  source 1..654: mol_type = other DNA

SEQ ID NO: 97 - DNA, 345 bp, synthetic construct; identical to SEQ ID NO: 77
  source 1..345: mol_type = other DNA

SEQ ID NO: 98 - DNA, 1335 bp, synthetic construct; identical to SEQ ID NO: 78
  source 1..1335: mol_type = other DNA

SEQ ID NO: 99 - DNA, 333 bp, synthetic construct; identical to SEQ ID NO: 91
  source 1..333: mol_type = other DNA

SEQ ID NO: 100 - DNA, 654 bp, synthetic construct; identical to SEQ ID NO: 92
  source 1..654: mol_type = other DNA

SEQ ID NO: 101 - DNA, 345 bp, synthetic construct; identical to SEQ ID NO: 81
  source 1..345: mol_type = other DNA

SEQ ID NO: 102 - DNA, 1335 bp, synthetic construct; identical to SEQ ID NO: 82
  source 1..1335: mol_type = other DNA

SEQ ID NO: 103 - DNA, 333 bp, synthetic construct; identical to SEQ ID NO: 91
  source 1..333: mol_type = other DNA

SEQ ID NO: 104 - DNA, 654 bp, synthetic construct; identical to SEQ ID NO: 92
  source 1..654: mol_type = other DNA

SEQ ID NO: 105 - DNA, 345 bp, synthetic construct; identical to SEQ ID NO: 85
  source 1..345: mol_type = other DNA

SEQ ID NO: 106 - DNA, 1335 bp, synthetic construct; identical to SEQ ID NO: 86
  source 1..1335: mol_type = other DNA

SEQ ID NO: 107 - DNA, 345 bp, synthetic construct; identical to SEQ ID NO: 69
  source 1..345: mol_type = other DNA

SEQ ID NO: 108 - DNA, 654 bp, synthetic construct; identical to SEQ ID NO: 92
  source 1..654: mol_type = other DNA

SEQ ID NO: 109 - DNA, 345 bp, synthetic construct; identical to SEQ ID NO: 69
  source 1..345: mol_type = other DNA

SEQ ID NO: 110 - DNA, 1335 bp, synthetic construct; identical to SEQ ID NO: 70
  source 1..1335: mol_type = other DNA

SEQ ID NO: 111 - DNA, 333 bp, synthetic construct
  source 1..333: mol_type = other DNA
gatatccagctgacccagagcccctcctccttgtctgcatccgtgggagacagagtgaccattacctgccgggcctccgagtctgtggactcttacgtgaacagcttcttgcactggtaccagcagaaacccggcaaagcccccaagctgctcatttacagggcctccaacctgcagtccggcgttccttctcgcttttccgggtccggttccaggaccgatttcaccctcaccatctcctccctgcagcccgaagatgttgctacctactactgccagcagtccaacgaggacccctacaccttcggccagggtactaagctggagatcaag

SEQ ID NO: 112 - DNA, 654 bp, synthetic construct
  source 1..654: mol_type = other DNA
gatatccagctgacccagagcccctcctccttgtctgcatccgtgggagacagagtgaccattacctgccgggcctccgagtctgtggactcttacgtgaacagcttcttgcactggtaccagcagaaacccggcaaagcccccaagctgctcatttacagggcctccaacctgcagtccggcgttccttctcgcttttccgggtccggttccaggaccgatttcaccctcaccatctcctccctgcagcccgaagatgttgctacctactactgccagcagtccaacgaggacccctacaccttcggccagggtactaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 113 - DNA, 345 bp, synthetic construct; identical to SEQ ID NO: 73
  source 1..345: mol_type = other DNA

SEQ ID NO: 114 - DNA, 1335 bp, synthetic construct; identical to SEQ ID NO: 74
  source 1..1335: mol_type = other DNA

SEQ ID NO: 115 - DNA, 333 bp, synthetic construct; identical to SEQ ID NO: 111
  source 1..333: mol_type = other DNA

SEQ ID NO: 116 - DNA, 654 bp, synthetic construct; identical to SEQ ID NO: 112
  source 1..654: mol_type = other DNA

SEQ ID NO: 117 - DNA, 345 bp, synthetic construct; identical to SEQ ID NO: 77
  source 1..345: mol_type = other DNA

SEQ ID NO: 118 - DNA, 1335 bp, synthetic construct; identical to SEQ ID NO: 78
  source 1..1335: mol_type = other DNA

SEQ ID NO: 119 - DNA, 333 bp, synthetic construct; identical to SEQ ID NO: 111
  source 1..333: mol_type = other DNA

SEQ ID NO: 120 - DNA, 654 bp, synthetic construct; identical to SEQ ID NO: 112
  source 1..654: mol_type = other DNA

SEQ ID NO: 121 - DNA, 345 bp, synthetic construct; identical to SEQ ID NO: 81
  source 1..345: mol_type = other DNA

SEQ ID NO: 122 - DNA, 1335 bp, synthetic construct; identical to SEQ ID NO: 82
  source 1..1335: mol_type = other DNA

SEQ ID NO: 123 - DNA, 333 bp, synthetic construct; identical to SEQ ID NO: 111
  source 1..333: mol_type = other DNA

SEQ ID NO: 124 - DNA, 654 bp, synthetic construct; identical to SEQ ID NO: 112
  source 1..654: mol_type = other DNA

SEQ ID NO: 125 - DNA, 345 bp, synthetic construct; identical to SEQ ID NO: 85
  source 1..345: mol_type = other DNA

SEQ ID NO: 126 - DNA, 1335 bp, synthetic construct; identical to SEQ ID NO: 86
  source 1..1335: mol_type = other DNA

SEQ ID NO: 127 - DNA, 333 bp, synthetic construct; identical to SEQ ID NO: 111
  source 1..333: mol_type = other DNA

SEQ ID NO: 128 - DNA, 654 bp, synthetic construct; identical to SEQ ID NO: 112
  source 1..654: mol_type = other DNA

SEQ ID NO: 129 - protein, 18 aa, synthetic construct
MHSSALLCCLVLLTGVRA

SEQ ID NO: 130 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 9 aa, synthetic construct
GYSITSDSA

SEQ ID NO: 132 - protein, 16 aa, synthetic construct
YISYSGSTSYNPSLKS

SEQ ID NO: 133 - protein, 8 aa, synthetic construct
VRGLRFAY

SEQ ID NO: 134 - protein, 10 aa, synthetic construct
ESVDSYVNSF

SEQ ID NO: 135 - protein, 7 aa, synthetic construct
RASNLQS

SEQ ID NO: 136 - protein, 9 aa, synthetic construct
QQSNEDPYT

SEQ ID NO: 137 - protein, 114 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARYFDYWGQGTLVTVSS

SEQ ID NO: 138 - protein, 112 aa, synthetic construct
QVQLQESGPGLVKPSETLSLTCTVSGGSISSHYWSWIRQPPGKGLEWIGYIYYSGSTNYNPSLKSRVTISVDTSKNQFSLKLSSVTAADTAVYYCARYFDYWGQGTLVTVSS

SEQ ID NO: 139 - protein, 114 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARYFDYWGQGTLVTVSS

SEQ ID NO: 140 - protein, 112 aa, synthetic construct
QVQLQESGPGLVKPSETLSLTCTVSGGSISSYYWSWIRQPPGKGLEWIGYIYTSGSTNYNPSLKSRVTISVDTSKNQFSLKLSSVTAADTAVYYCARYFDYWGQGTLVTVSS

SEQ ID NO: 141 - protein, 114 aa, synthetic construct
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCARYFDYWGQGTLVTVSS

SEQ ID NO: 142 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPYTFGQGTKLEIK

SEQ ID NO: 143 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 144 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 145 - protein, 111 aa, synthetic construct
DIVLTQSPASLAVSLGQRATISCRASESVDSYVDSFLHWYQQKPGQPPKLLIYRASNLQSGIPARFSGSGSRTDFTLTINPVEADDVATYYCQQSNEDPYTFGGGTKLEIK

SEQ ID NO: 146 - protein, 111 aa, synthetic construct
DIVLTQSPASLAVSLGQRATISCRASESVDSYVSSFLHWYQQKPGQPPKLLIYRASNLQSGIPARFSGSGSRTDFTLTINPVEADDVATYYCQQSNEDPYTFGGGTKLEIK

SEQ ID NO: 147 - protein, 111 aa, synthetic construct
DIVLTQSPASLAVSLGQRATISCRASESVDSYVTSFLHWYQQKPGQPPKLLIYRASNLQSGIPARFSGSGSRTDFTLTINPVEADDVATYYCQQSNEDPYTFGGGTKLEIK

SEQ ID NO: 148 - protein, 111 aa, synthetic construct
DIVLTQSPASLAVSLGQRATISCRASESVDSYVNAFLHWYQQKPGQPPKLLIYRASNLQSGIPARFSGSGSRTDFTLTINPVEADDVATYYCQQSNEDPYTFGGGTKLEIK

SEQ ID NO: 149 - protein, 111 aa, synthetic construct
DIVLTQSPASLAVSLGQRATISCRASESVDSYVQSFLHWYQQKPGQPPKLLIYRASNLQSGIPARFSGSGSRTDFTLTINPVEADDVATYYCQQSNEDPYTFGGGTKLEIK

SEQ ID NO: 150 - protein, 107 aa, synthetic construct [sequence omitted]

SEQ ID NO: 151 - protein, 111 aa, synthetic construct
DIQLTQSPSSLSASVGDRVTITCRASESVDSYVDSFLHWYQQKPGKAPKLLIYRASNLQSGVPSRFSGSGSRTDFTLTISSLQPEDVATYYCQQSNEDPYTFGQGTKLEIK

SEQ ID NO: 152 - protein, 111 aa, synthetic construct
DIQLTQSPSSLSASVGDRVTITCRASESVDSYVSSFLHWYQQKPGKAPKLLIYRASNLQSGVPSRFSGSGSRTDFTLTISSLQPEDVATYYCQQSNEDPYTFGQGTKLEIK

SEQ ID NO: 153 - protein, 111 aa, synthetic construct
DIQLTQSPSSLSASVGDRVTITCRASESVDSYVTSFLHWYQQKPGKAPKLLIYRASNLQSGVPSRFSGSGSRTDFTLTISSLQPEDVATYYCQQSNEDPYTFGQGTKLEIK

SEQ ID NO: 154 - protein, 111 aa, synthetic construct
DIQLTQSPSSLSASVGDRVTITCRASESVDSYVNAFLHWYQQKPGKAPKLLIYRASNLQSGVPSRFSGSGSRTDFTLTISSLQPEDVATYYCQQSNEDPYTFGQGTKLEIK

SEQ ID NO: 155 - protein, 111 aa, synthetic construct
DIQLTQSPSSLSASVGDRVTITCRASESVDSYVQSFLHWYQQKPGKAPKLLIYRASNLQSGVPSRFSGSGSRTDFTLTISSLQPEDVATYYCQQSNEDPYTFGQGTKLEIK

SEQ ID NO: 156 - DNA, 92 bp, synthetic construct
  source 1..92: mol_type = other DNA
gcggccgcaaactacaagacagacttgcaaaagaaggcatgcacagctcagcactgctctgttgcctggtcctcctgactggggtgagggcc

SEQ ID NO: 157 - DNA, 10 bp, synthetic construct
  source 1..10: mol_type = other DNA
tgattctaga

SEQ ID NO: 158 - protein, 9 aa, synthetic construct
GGSISSDSA

(19) United States
(12) Patent Application Publication
(10) Pub. No.: US 2025/0011456 A1
(43) Pub. Date: Jan. 9, 2025

(54) ANTIBODIES AGAINST CD14 AND USES THEREOF

(71) Applicant: Villanelle Life Science Inc, Houston, TX (US)

(72) Inventors: Xuanye CAO, Houston, TX (US); Siyu XIAO, Houston, TX (US)

(21) Appl. No.: 18/762,549

(22) Filed: Jul. 2, 2024

Related U.S. Application Data
(60) Provisional application No. 63/512,570, filed on Jul. 7, 2023.

(51) Int. Cl. C07K 16/28; A61K 39/00; A61K 45/06; A61P 35/00

(52) U.S. Cl. CPC C07K 16/2896; A61K 45/06; A61P 35/00; A61K 2039/505; C07K 2317/24; C07K 2317/565; C07K 2317/92

(57) ABSTRACT

Embodiments described herein generally relate to antibodies against CD14, and uses of such antibodies, in particular their use in the treatment of cancers. Also described herein are nucleic acid molecules including nucleotide sequences encoding the antibodies, vectors including the nucleic acid molecules, and host cells including the nucleic acid molecules or the vectors. Also described herein are compositions, pharmaceutical compositions, and uses of such compositions and pharmaceutical compositions.

### CROSS-REFERENCE TO RELATED APPLICATIONS

This application claims benefit of and priority to U.S. Provisional Patent Application No. 63/512,570, filed on Jul. 7, 2023, which is incorporated herein by reference in its entirety.

### REFERENCE TO A SEQUENCE LISTING

This application contains references to amino acid and nucleic acid sequences which have been submitted as the sequence listing text file entitled “SEQ ID NOs 1-158”, file size 196 KiloBytes (KB), created Jul. 1, 2024, which is hereby incorporated by reference in its entirety.

### FIELD

Embodiments described herein generally relate to antibodies against CD14, and uses of such antibodies, in particular, their use in the treatment of cancers.

### BACKGROUND

Cluster of Differentiation 14 (CD14), a lipopolysaccharide-binding protein, belongs to a family of leukocyte differentiation antigens expressed on the surface of leukocytes. CD14 has been found to play a role in various diseases including sepsis, hematological diseases, coronary heart disease, cancer, diabetes, rheumatoid arthritis, and Alzheimer's disease, among other diseases. A widely studied aspect of CD14 is its relationship with inflammation. In the context of inflammation, researchers have proposed its role in processes of lipopolysaccharide (LPS)-mediated cellular responses. Here, LPS first binds to LPS binding protein (LBP), forming a LPS-LBP complex. This complex then binds to CD14, generating an LPS-LBP-CD14 complex, and initiating further signal transduction and the secretion of inflammatory factors.

CD14 has a high degree of homology between different species, with studies showing that human and mouse CD14 share up to 65% homology. In the human body, CD14 exists in two forms: membrane-bound CD14 (mCD14) and soluble CD14 (sCD14). Both have similar functions, but have different structures and are found in different locations. mCD14 is located on the surface of monocytes and is a 55 kiloDalton (kDa) glycoprotein that is anchored to the cell surface by glycosylphosphatidylinositol (GPI). It is a non-transmembrane protein that cannot transmit signals into the cell itself. sCD14 is found in human serum and urine and cannot attach to cell surfaces due to its lack of a hydrophobic C-terminal region. sCD14 is generated by various mechanisms including bypassing the addition of GPI during formation, cleavage of mCD14 by phospholipase D, and direct protein hydrolysis from the cell surface. In human plasma, sCD14 exists in various forms with molecular weights ranging from 40 kDa to 53 kDa. In various pathological conditions, such as psoriasis and systemic lupus erythematosus, levels of SCD14 increase during chronic inflammatory responses. Accordingly, CD14 is a target for anti-inflammatory drug development and a diagnostic marker for various diseases. Additionally, CD14 interacts with other pathogenic components of both microbial and non-microbial origins, mediating the development of other diseases. As a result, inhibiting the expression of CD14 in the body can be utilized for combating various diseases, including autoimmune disorders and cancers.

There is a need for antibodies against CD14 and for uses of such antibodies.

### SUMMARY

Embodiments described herein generally relate to antibodies against CD14, and uses of such antibodies, in particular, their use in the treatment of cancers. The present disclosure provides novel antibodies targeting CD14 or antigen binding fragments thereof. A variety of functional assays described herein demonstrate the potent anti-tumor effect of the antibodies. Conventional technologies have failed when it comes to the treatment of various cancers such as bladder cancer and recurrent bladder cancer.

In an embodiment, the present disclosure provides an antibody specifically binding to CD14, or an antigen binding fragment thereof, including a light chain variable region (VL) and a heavy chain variable region (VH), wherein the VH includes HCDRs 1-3 having amino acid sequences as set forth in SEQ ID NOs: 131-133, respectively, and the VL includes LCDRs 1-3 having amino acid sequences as set forth in SEQ ID NOs: 134-136 respectively.

Implementations may include one or more of the following. The VH can include an amino acid sequence as set forth in one or more of SEQ ID NOs: 1, 17, 21, 37, 41, 57, or 61, or an amino acid sequence having at least 80% sequence identity to one or more of SEQ ID NOs: 1, 17, 21, 37, 41, 57, or 61; and the VL includes an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63. The antibody can be of an isotype that includes one or more of IgG, IgA, IgM, IgE, or IgD. The antibody can be of a subtype that includes one or more of IgG1, IgG2, or IgG3. The antigen binding fragment can include one or more of Fab, Fab′, F(ab′)₂, Fv, scFv, or ds-scFv. The antibody can be a monoclonal antibody. The antibody can include a heavy chain including an amino acid sequence as set forth in one or more of SEQ ID NOs: 2, 18, 22, 38, 42, 58, or 62, or an amino acid sequence having at least 80% sequence identity to one or more of SEQ ID NOs: 2, 18, 22, 38, 42, 58, or 62; and a light chain including an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

In another embodiment, the present disclosure provides an antibody specifically binding to CD14, or an antigen binding fragment thereof, comprising: a heavy chain variable region (VH) comprising HCDRs 1-3 having amino acid sequences as set forth in SEQ ID NOs: 158, 132, and 133, respectively; and a light chain variable region (VL) comprising LCDRs 1-3 having amino acid sequences set forth in SEQ ID NOs: 134-136, respectively.

Implementations may include one or more of the following. The VH can include an amino acid sequence as set forth in one or more of SEQ ID NOs: 5, 9, 13, 25, 29, 33, 45, 49, or 53, or an amino acid sequence having at least 80% sequence identity to one or more of SEQ ID NOs: 5, 9, 13, 25, 29, 33, 45, 49, or 53. The VL can include an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63. The antibody can be of an isotype that includes one or more of IgG, IgA, IgM, IgE, or IgD. The antibody can be of a subtype that includes one or more of IgG1, IgG2, or IgG3. The antigen binding fragment can include one or more of Fab, Fab′, F(ab′)₂, Fv, scFv, or ds-scFv. The antibody can be a monoclonal antibody. The antibody can include a heavy chain including an amino acid sequence as set forth in one or more of SEQ ID NOs: 6, 10, 14, 26, 30, 34, 46, 50, or 54, or an amino acid sequence having at least 80% sequence identity to one or more of SEQ ID NOs: 6, 10, 14, 26, 30, 34, 46, 50, or 54; and a light chain including an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

In another embodiment, the present disclosure provides a nucleic acid molecule including a nucleotide sequence encoding an antibody or an antigen binding fragment thereof described herein.

In another embodiment, the present disclosure provides a vector including the nucleic acid molecule described herein.

In another embodiment, the present disclosure provides a host cell including the nucleic acid molecule described herein or the vector described herein.

In another embodiment, the present disclosure provides a pharmaceutical composition including (i) an antibody or an antigen binding fragment thereof described herein, and (ii) a pharmaceutically acceptable carrier or excipient.

In another embodiment, the present disclosure provides a pharmaceutical composition including (i) an antibody or an antigen binding fragment thereof described herein, and (ii) a second therapeutic agent. The second therapeutic agent can include one or more of an antibody, a chemotherapeutic agent, or a small molecule drug. The small molecule drug can include a small molecule cytotoxic agent. The small molecule cytotoxic agent can include cisplatin.

In another embodiment, the present disclosure provides a method of treating a cancer in a subject (for example, a patient) including administering to the subject an effective amount of an antibody or an antigen binding fragment thereof described herein, a pharmaceutical composition described herein, or combinations thereof.

Implementations may include one or more of the following. The cancer can be a CD14 positive cancer. The cancer can include one or more of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, or combinations thereof. The cancer can include bladder cancer, recurrent bladder cancer, or combinations thereof. The method can further include administering to the subject a second therapeutic agent. The second therapeutic agent can include one or more of an antibody, a chemotherapeutic agent, or a small molecule drug.

In another embodiment, the present disclosure provides use of an antibody or an antigen binding fragment thereof described herein or a pharmaceutical composition described herein in the manufacture of a medicament for treating a cancer in a subject. Implementations may include one or more of the following. The cancer can be a CD14 positive cancer. The cancer can include one or more of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, or combinations thereof. The cancer can include bladder cancer, recurrent bladder cancer, or combinations thereof.

In another embodiment, the present disclosure provides an antibody or an antigen binding fragment thereof described herein or a pharmaceutical composition described herein for use in treating a cancer in a subject. Implementations may include one or more of the following. The cancer can be a CD14 positive cancer. The cancer can include one or more of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, or combinations thereof. The cancer can include bladder cancer, recurrent bladder cancer, or combinations thereof.

In another embodiment, the present disclosure provides a combination therapy including (i) an antibody or an antigen binding fragment thereof described herein, and (ii) optionally a second therapeutic agent. Implementations can include one or more of the following. The therapy can be administered over a 28-day cycle. In some embodiments, the second therapeutic agent can include one or more of an antibody, a chemotherapeutic agent, a small molecule drug, or combinations thereof. The combination therapy can include a dosing regimen that includes: (a) administering an antibody or an antigen binding fragment thereof described herein every 3 or 4 days on days 1-28 of a first 28-day cycle; and (b) administering the second therapeutic agent every 3 or 4 days on days 1-28 of a first 28-day cycle. The small molecule drug can be a small molecule cytotoxic agent. The antibody or the antigen binding fragment thereof described herein can be administered at an amount of 5 mg/kg to about 40 mg/kg, such as from about 5 mg/kg to about 25 mg/kg, such as from about 5 mg/kg to about 15 mg/kg. Administration of the antibody or the antigen binding fragment thereof can be performed by intravenous (IV) infusion. The second therapeutic agent can be administered at an amount of 1 mg/kg to about 40 mg/kg, such as from about 1 mg/kg to about to 15 mg/kg, such as from about 1 mg/kg to about 10 mg/kg such as from about 3 mg/kg to about 6 mg/kg. Administration of the second therapeutic agent can be performed by IV infusion.

In another embodiment, the present disclosure provides a method of treating a cancer in a subject including administering to the subject an effective amount of a combination therapy including (i) an antibody or an antigen binding fragment thereof described herein, and (ii) a second therapeutic agent. Implementations can include one or more of the following. The second therapeutic agent can include one or more of an antibody, a chemotherapeutic agent, and a small molecule drug. The cancer can include a CD14 positive cancer. The cancer can include one or more of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, or combinations thereof.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIGS. 1A-1C show example pathological images illustrating instances of recurrent bladder cancer tumors that exhibit a high expression level of CD14 according to at least one embodiment of the present disclosure.

FIG. 2 shows immunohistochemistry staining of CD14 expression on patient-derived xenograft (PDX) slides according to at least one embodiment of the present disclosure.

FIG. 3 shows data for tumor volume under different treatment regimens according to at least one embodiment of the present disclosure.

FIG. 4 shows data for tumor weight after 28 days of different treatment regimens according to at least one embodiment of the present disclosure.

FIG. 5 shows images of tumors collected from PDX mice treated with different treatment regimens according to at least one embodiment of the present disclosure.

FIG. 6A shows images of tumors collected from PDX mice treated with different concentrations of Anti-CD14-IgG1 according to at least one embodiment of the present disclosure.

FIG. 6B shows data for tumor volume of PDX mice treated with various concentrations of Anti-CD14-IgG1 according to at least one embodiment of the present disclosure.

FIG. 6C shows data for tumor weight of PDX mice treated with various concentrations of Anti-CD14-IgG1 according to at least one embodiment of the present disclosure.

FIGS. 7A-7P show exemplary dynamic binding curves as a function of residence time with the antigen fully saturated according to at least one embodiment of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments described herein generally relate to antibodies against CD14, and uses of such antibodies, in particular, their use in the treatment of cancers. The present disclosure provides novel antibodies targeting CD14 or antigen binding fragments thereof. A variety of functional assays described herein demonstrate the potent anti-tumor effect of the antibodies. Conventional technologies have failed when it comes to the treatment of various cancers such as bladder cancer and recurrent bladder cancer.

The use of headings is for purposes of convenience only and does not limit the scope of the present disclosure. Embodiments described herein can be combined with other embodiments. For example, features and elements of one described embodiment may be combined with other embodiments within the scope of the present disclosure.

Unless indicated or defined otherwise, terms used have their usual meaning in the art, which is clear to those skilled in the art.

As used herein, the term “antibody” refers to an immunoglobulin molecule which has the ability to specifically bind to a specific antigen. An antibody often includes a variable region and a constant region in each of a heavy chain and a light chain. The variable regions of the heavy and light chains of antibodies contain a binding domain that interacts with an antigen. The constant regions of antibodies may mediate the binding of the immunoglobulin to host tissues or factors, including various cells of the immune system (for example, effector cells) and components of the complement system such as C1q, the first component in the classical pathway of complement activation. Accordingly, most antibodies include a heavy chain variable region (VH) and a light chain variable region (VL) that together form the portion of the antibody that binds to the antigen.

A “light chain variable region” (VL) or “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 1989, 342, 878-883); a composite of Chothia Kabat CDR in which CDR-HI is a composite of Chothia and Kabat CDRs; the AbM definition used by Oxford Molecular's antibody modeling software; the CONTACT definition of Martin et al. (world wide web bioinfo.org.uk/abs); and the IMGT definition IMGT (IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33: D593-597 (2005); Lefranc, M.-P. et al., Nucleic Acids Res., 37: D1006-1012 (2009); Lefranc, M.-P. et al., Nucleic Acids Res., 43: D413-422 (2015)). The present disclosure uses CDRs defined according to the Kabat numbering system.

As used herein, the term “antibody” should be understood in its broadest meaning, and includes monoclonal antibodies (including full-length monoclonal antibodies), polyclonal antibodies, antibody fragments, and multi-specific antibodies containing at least two different antigen binding regions (for example, bispecific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

As used herein, the term “antigen binding fragment” of an antibody refers to one or more fragments of an antibody that retain the ability to specifically bind to an antigen (for example, a CD14 protein). It has been shown that the antigen binding function of an antibody can be performed by fragments of a full-length antibody. Examples of antigen binding fragments encompassed within the term “antigen binding portion” of an antibody include (i) a Fab fragment, a monovalent fragment that includes VL, VH, CL and CHI domains; (ii) a F(ab′)₂ fragment, a bivalent fragment comprising two Fab fragments linked by a disulfide bridge at the hinge region; (iii) a Fab′ fragment, which is essentially an Fab with part of the hinge region (Fundamental Immunology, W. E. Paul ed., 3rd edition, 1993); (iv) a Fd fragment that includes VH and CH1 domains; (v) a Fd′ fragment having VH and CH1 domains and one or more cysteine residues at the C-terminus of the CH1 domain; (vi) a Fv fragment that includes VL and VH domains of a single arm of an antibody, (vii) a dAb fragment (E. Ward et al., Nature 341, 544-546 (1989)), which consists of a VH domain; (viii) an isolated complementarity determining region (CDR); and (ix) a nanobody, a heavy chain variable region containing a single variable domain and two constant domains. Further, although the two domains of the Fv fragment, VL and VH are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen binding fragment” of an antibody. Further, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity. These antigen binding fragments can be obtained using conventional techniques known to those with skill in the art, and the fragments are screened for utility in the same manner as are intact antibodies.

As used herein, the term “binding” or “specifically binding” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant and an antigen-binding site on the antibody: the lesser the value of the KD, the stronger the binding strength between an antigenic determinant and the antibody. Alternatively, the affinity can also be expressed as the affinity constant (KA), which is 1/KD. Avidity is the measure of the strength of binding between an antibody and the pertinent antigen. Avidity is related to both the affinity between an antigenic determinant and its antigen binding site on the antibody and the number of pertinent binding sites present on the antibody. Typically, an antibody will bind to an antigen with a dissociation constant (KD) of 10⁻⁵ M to 10⁻¹² M or less, such as 10⁻⁷ M to 10⁻¹² M or less, for example 10⁻⁸ M to 10⁻¹² M, and/or with a binding affinity of at least 10⁷ M⁻¹, such as 10⁸ M⁻¹, for example, at least 10⁹ M⁻¹, such as at least 10¹² M⁻¹. Any K_{D} value greater than 10⁻⁴ M is generally considered to indicate non-specific binding. Specific binding of an antibody to an antigen or antigenic determinant can be determined in any suitable manner known, including, for example, Scatchard analysis and/or competitive binding assays, such as sandwich competition assays, radioimmunoassays (RIA), enzyme immunoassays (EIA) and variants thereof known in the art.

As used herein, the term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes 3 or more, and more usually, at least 5 amino acids or 8-10 amino acids in a spatial conformation. The epitope defines the smallest binding site of an antibody and therefore is the specific target of the antibody or antigen binding fragment thereof.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence described in SEQ ID NO: Y. The degree of sequence identity between two or more nucleotide sequences may be calculated using a known computer algorithm for sequence alignment such as NCBI BLAST, using standard settings. In determining the degree of sequence identity between two amino acid sequences, a skilled artisan can consider “conservative” amino acid substitutions, which can be described as amino acid substitutions in which an amino acid residue is replaced with another amino acid residue of similar chemical structure and which has little or essentially no influence on the function, activity, or other biological properties of the polypeptide. Such conservative amino acid substitutions are known, for example from WO 2004/037999, WO 1998/049185, WO 2001/009300, and GB 2 357 768-A; and certain types and/or combinations of such substitutions may be selected on the basis of the pertinent teachings from WO 2004/037999 and WO 1998/049185, as well as from the further references cited therein.

Such conservative substitutions can be substitutions in which one amino acid within the following groups (a)-(c) is substituted by another amino acid residue within the same group: (a) small aliphatic, nonpolar or slightly polar residues: alanine (Ala), serine (Ser), threonine (Thr), proline (Pro), and glycine (Gly); (b) polar, negatively charged residues and their (uncharged) amides: aspartic acid (Asp), asparagine (Asn), glutamic acid (Glu), and glutamine (Gln); (c) polar, positively charged residues: histidine (His), arginine (Arg), and lycine (Lys); (d) large aliphatic, nonpolar residues: methionine (Met), leucine (Leu), isoleucine (Ile), valine (Val), and cysteine (Cys); and (c) aromatic residues: phenylalanine (Phe), tyrosine (Tyr), and tryptophan (Trp).

Particularly suitable conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp; and/or Phe into Val, into Ile, or into Leu.

Any suitable amino acid substitutions applied to polypeptides described herein can also be based on the analysis of the frequencies of amino acid variations between homologous proteins of G. E. Schulz and R. H. Schirmer, Principles of Protein Structure, Springer, New York, 1978, on the analyses of structure forming potentials developed by P. Y. Chou and G. D. Fasman, Biochemistry, 1974, 13, 211, and Adv. Enzymol., A. Meister ed., J. Wiley and Sons, 1978, 47, 45-149 and on the analysis of hydrophobicity patterns in proteins developed by D. Eisenberg et al., Proc. Nat. Acad. Sci. USA, 1984, 81, 140-144; J. Kyte and R. F. Doolittle, J. Mol. Biol., 1982, 157, 105-132, and D. M. Engelman et al., Annu. Rev. Biophys. Biophys. Chem., 1986, 15, 321-53, each of which are incorporated herein in their entirety by reference.

For purposes of the present disclosure, the term “monoclonal antibody” refers to an antibody obtained from a substantially homogeneous antibody population. That is, each antibody(ies) constituting the population are the same, except for possible naturally occurring mutations in a small amount. Monoclonal antibodies are highly specific and are directed against a single antigen. As used herein, the term “monoclonal antibody” is not limited to antibodies produced by hybridoma technology, and should not be interpreted as requiring production of antibodies by any specific method.

For purposes of the present disclosure, the term “tumor associated antigen” refers to an antigen that is differentially expressed in cancer cells compared to normal cells, and therefore can be used to target cancer cells.

For purposes of the present disclosure, the term “CD14” refers to the human CD14 protein complex. CD14 is a monomeric glycoprotein having a single polypeptide chain. The term includes any suitable CD14 variant, isoform, and species homolog which are naturally expressed by cells, or are expressed on cells transfected with genes or cDNA encoding the aforementioned chains.

As used herein, the term “vector” refers to a nucleic acid molecule capable of transporting another nucleic acid to which it has been linked. As used herein, the term “host cell” refers to a cell into which an expression vector has been introduced.

As used herein, the term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans. For purposes of the present disclosure, the terms “treatment”, “treat”, “treating”, and similar terms refer to administering an agent, or carrying out a procedure, for the purposes of obtaining an effect. The effect can be prophylactic in terms of completely or partially preventing a disease or symptom thereof and/or can be therapeutic in terms of effecting a partial or complete cure for a disease and/or symptoms of the disease.

For purposes of the present disclosure, the term “treatment” can include treatment of a disease or disorder (for example) in a mammal, such as a human, and includes: (a) preventing the disease or a symptom of a disease from occurring in a subject which may be predisposed to the disease but has not yet been diagnosed as having it (for example, including diseases that may be associated with or caused by a primary disease); (b) inhibiting the disease, for example, arresting its development; and (c) relieving the disease, for example, causing regression of the disease. Treating can refer to any suitable indicia of success in the treatment, amelioration, or prevention of a cancer, including any suitable objective parameter or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective parameters, one or more subjective parameters, or combinations thereof; including the results of an examination by a physician. Accordingly, the term “treating” includes the administration of antibodies described herein, compositions described herein, combination therapies described herein, etc. to alleviate, to arrest, or inhibit development of the symptoms or conditions associated with diseases (for example, cancers).

For purposes of the present disclosure, the term “subject” refers to any suitable mammalian subject for whom diagnosis, treatment or therapy is desired. The terms “subject” and “patient” are used interchangeably unless the context indicates otherwise. For purposes of the present disclosure, the term “mammal” for purposes of treatment refers to any suitable animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, among others.

As used herein, the term “therapeutic effect” refers to the reduction, elimination, or prevention of the disease, symptoms of the disease, or side effects of the disease in the subject. As used herein, the term “effective amount” refers to the amount that, when administered to a subject for treating a disease, is sufficient to effect treatment for that disease.

As used herein, the term “small molecule” refers to compounds, whether naturally occurring or artificially created (e.g., via chemical synthesis) that have relatively low molecular weight and that are different from proteins, polypeptides, or nucleic acid molecules described herein. Typically, small molecules have a molecular weight of less than about 1,500 g/mol. Non-limiting examples include doxorubicin and cisplatin.

Embodiments of the present disclosure generally relate to an antibody or an antigen binding fragment thereof specifically binding to CD14. The antibody or the antigen binding fragment thereof can include a heavy chain variable region (VH) and a light chain variable region (VL). The VH can include an amino acid sequence as set forth in one or more of SEQ ID NOs: 1, 5, 9, 13, 17, 21, 25, 29, 33, 37, 41, 45, 49, 53, 57, or 61. The VL can include an amino acid sequence as set forth in one or more of SEQ ID NOs: 3, 7, 11, 15, 19, 23, 27, 31, 35, 39, 43, 47, 51, 55, 59, or 63.

In some embodiments, which may be combined with other embodiments, the VH can comprise or consist of an amino acid sequence having at least 80%, at least 85%, at least 90%, at least 95%, at least 98%, at least 99%, or 100% sequence identity to SEQ ID NOs: 1, 5, 9, 13, 17, 21, 25, 29, 33, 37, 41, 45, 49, 53, 57, or 61; and/or the VL can comprise or consist of an amino acid sequence having at least 80%, at least 85%, at least 90%, at least 95%, at least 98%, at least 99%, or 100% sequence identity to SEQ ID NOs: 3, 7, 11, 15, 19, 23, 27, 31, 35, 39, 43, 47, 51, 55, 59, or 63.

In some embodiments, which may be combined with other embodiments, CDR sequences are defined according to the Kabat numbering system. When CDR sequences are defined according to the Kabat numbering system, the VH of an antibody of the present disclosure can include HCDRs 1-3 having the amino acid sequences as set forth in SEQ ID NOs: 131-133, respectively; and the VL of an antibody of the present disclosure can include: LCDRs 1-3 having the amino acid sequences as set forth in SEQ ID NOs: 134-136, respectively.

In some embodiments, which may be combined with other embodiments, and when CDR sequences are defined according to the Kabat numbering system, the VH of an antibody of the present disclosure can include HCDRs 1-3 having the amino acid sequence as set forth in SEQ ID NOs: 158, 132, and 133, respectively; and the VL of an antibody of the present disclosure can include: LCDRs 1-3 having the amino acid sequences as set forth in SEQ ID NOs: 134-136, respectively.

The VH can comprise or consist of a functional variant of an amino acid sequence formed by insertion, deletion, substitution, or combinations thereof, of one or more amino acid(s) therein, provided that the functional variant retains the ability of binding to CD14. In some embodiments, which may be combined with other embodiments, the VH can comprise or consist of a functional variant of the amino acid sequence as set forth in one or more of SEQ ID NOs: 1, 5, 9, 13, 17, 21, 25, 29, 33, 37, 41, 45, 49, 53, 57, or 61 formed by insertion, deletion, substitution, or combinations thereof, of one or more amino acid(s) therein, provided that the functional variant retains the ability of binding to CD14.

The VL can comprise or consist of a functional variant of an amino acid sequence formed by insertion, deletion, substitution, or combinations thereof, of one or more amino acid(s) therein, provided that the functional variant retains the ability of binding to CD14. In some embodiments, which may be combined with other embodiments, the VL can comprise or consist of a functional variant of an amino acid sequence as set forth in one or more of SEQ ID NOs: 3, 7, 11, 15, 19, 23, 27, 31, 35, 39, 43, 47, 51, 55, 59, or 63 formed by insertion, deletion and/or substitution of one or more amino acid(s) therein, provided that the functional variant retains the ability of binding to CD14.

The functional variant can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence of the parent polypeptide.

For example, a functional variant of SEQ ID NO: 1 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1. This applies to functional variants of other SEQ ID NOs described herein. For example, a functional variant of SEQ ID NO: 1, 5, 9, 13, 17, 21, 25, 29, 33, 37, 41, 45, 49, 53, 57, or 61 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1, 5, 9, 13, 17, 21, 25, 29, 33, 37, 41, 45, 49, 53, 57, or 61, respectively.

As another example, a functional variant of SEQ ID NO: 3 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3. This applies to functional variants of other SEQ ID NOs described herein. For example, a functional variant of SEQ ID NO: 3, 7, 11, 15, 19, 23, 27, 31, 35, 39, 43, 47, 51, 55, 59, or 63 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3, 7, 11, 15, 19, 23, 27, 31, 35, 39, 43, 47, 51, 55, 59, or 63, respectively.

In some embodiments, which may be combined with other embodiments, a functional variant of SEQ ID NO: 1 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1 and formed by insertion, deletion and/or substitution of one or more amino acid(s) in SEQ ID NO: 1. This applies to functional variants of other SEQ ID NOs described herein. For example, a functional variant of SEQ ID NO: 1, 5, 9, 13, 17, 21, 25, 29, 33, 37, 41, 45, 49, 53, 57, or 61 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1, 5, 9, 13, 17, 21, 25, 29, 33, 37, 41, 45, 49, 53, 57, or 61, respectively, and formed by insertion, deletion and/or substitution of one or more amino acid(s) in SEQ ID NO: 1, 5, 9, 13, 17, 21, 25, 29, 33, 37, 41, 45, 49, 53, 57, or 61, respectively.

In some embodiments, which may be combined with other embodiments, the functional variant of SEQ ID NO: 3 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3 and formed by insertion, deletion and/or substitution of one or more amino acid(s) in SEQ ID NO: 3. This applies to functional variants of other SEQ ID NOs described herein. For example, a functional variant of SEQ ID NO: 3, 7, 11, 15, 19, 23, 27, 31, 35, 39, 43, 47, 51, 55, 59, or 63 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3, 7, 11, 15, 19, 23, 27, 31, 35, 39, 43, 47, 51, 55, 59, or 63, respectively, and formed by insertion, deletion and/or substitution of one or more amino acid(s) in SEQ ID NO: 3, 7, 11, 15, 19, 23, 27, 31, 35, 39, 43, 47, 51, 55, 59, or 63, respectively.

In the context of the functional variant, the number of inserted, deleted, and/or substituted amino acid can be no more than 40% of the total number of amino acids in the parent amino acid sequence, such as no more than 35%, such as 1-33%, such as 5-30%, such as 10-25%, such as 15-20%. For example, the number of inserted, deleted and/or substituted amino acids can be 1-20 amino acids, such as 1-10 amino acids, such as 1-7 amino acids, such as 1-5 amino acids, such as 1-2 amino acids. The number of inserted, deleted, and/or substituted amino acids can be 1, 2, 3, 4, 5, 6, or 7 amino acids. The insertion, deletion and/or substitution can be performed at framework (FR) regions, for example, at FR1, FR2, FR3, and/or FR4.

The substitution of one or more amino acid(s) can be a conservative substitution of one or more amino acid(s). Such conservative substitutions can be substitutions in which one amino acid within the following groups (a)-(e) is substituted by another amino acid residue within the same group: (a) small aliphatic, nonpolar or slightly polar residues: Ala, Ser, Thr, Pro, and Gly; (b) polar, negatively charged residues and their (uncharged) amides: Asp, Asn, Glu, and Gln; (c) polar, positively charged residues: His, Arg, and Lys; (d) large aliphatic, nonpolar residues: Met, Leu, He, Val, and Cys; and (e) aromatic residues: Phe, Tyr, and Trp.

Conservative substitutions can include one or more of the following: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp; and/or Phe into Val, into Ile, or into Leu.

In some embodiments, the VH comprises, consists essentially of, or consists of an amino acid sequence as set forth in SEQ ID NO: 1; and the VL comprises, consists essentially of, or consists of an amino acid sequence as set forth in SEQ ID NO: 3.

In some embodiments, the VH comprises, consists essentially of, or consists of an amino acid sequence as set forth in SEQ ID NO: 5; and the VL comprises, consists essentially of, or consists of an amino acid sequence as set forth in SEQ ID NO: 7.

In some embodiments, the VH comprises, consists essentially of, or consists of an amino acid sequence as set forth in SEQ ID NO: 9; and the VL comprises, consists essentially of, or consists of an amino acid sequence as set forth in SEQ ID NO: 11.

In some embodiments, the VH comprises, consists essentially of, or consists of an amino acid sequence as set forth in SEQ ID NO: 13; and the VL comprises, consists essentially of, or consists of an amino acid sequence as set forth in SEQ ID NO: 15.

In some embodiments, the VH comprises, consists essentially of, or consists of an amino acid sequence as set forth in SEQ ID NO: 17; and the VL comprises, consists essentially of, or consists of an amino acid sequence as set forth in SEQ ID NO: 19.

In some embodiments, the VH comprises, consists essentially of, or consists of an amino acid sequence as set forth in SEQ ID NO: 21; and the VL comprises, consists essentially of, or consists of an amino acid sequence as set forth in SEQ ID NO: 23.

In some embodiments, the VH comprises, consists essentially of, or consists of an amino acid sequence as set forth in SEQ ID NO: 25; and the VL comprises, consists essentially of, or consists of an amino acid sequence as set forth in SEQ ID NO: 27.

In some embodiments, the VH comprises, consists essentially of, or consists of an amino acid sequence as set forth in SEQ ID NO: 29; and the VL comprises, consists essentially of, or consists of an amino acid sequence as set forth in SEQ ID NO: 31.

In some embodiments, the VH comprises, consists essentially of, or consists of an amino acid sequence as set forth in SEQ ID NO: 33; and the VL comprises, consists essentially of, or consists of an amino acid sequence as set forth in SEQ ID NO: 35.

In some embodiments, the VH comprises, consists essentially of, or consists of an amino acid sequence as set forth in SEQ ID NO: 37; and the VL comprises, consists essentially of, or consists of an amino acid sequence as set forth in SEQ ID NO: 39.

In some embodiments, the VH comprises, consists essentially of, or consists of an amino acid sequence as set forth in SEQ ID NO: 41; and the VL comprises, consists essentially of, or consists of an amino acid sequence as set forth in SEQ ID NO: 43.

In some embodiments, the VH comprises, consists essentially of, or consists of an amino acid sequence as set forth in SEQ ID NO: 45; and the VL comprises, consists essentially of, or consists of an amino acid sequence as set forth in SEQ ID NO: 47.

In some embodiments, the VH comprises, consists essentially of, or consists of an amino acid sequence as set forth in SEQ ID NO: 49; and the VL comprises, consists essentially of, or consists of an amino acid sequence as set forth in SEQ ID NO: 51.

In some embodiments, the VH comprises, consists essentially of, or consists of an amino acid sequence as set forth in SEQ ID NO: 53; and the VL comprises, consists essentially of, or consists of an amino acid sequence as set forth in SEQ ID NO: 55.

In some embodiments, the VH comprises, consists essentially of, or consists of an amino acid sequence as set forth in SEQ ID NO: 57; and the VL comprises, consists essentially of, or consists of an amino acid sequence as set forth in SEQ ID NO: 59.

In some embodiments, the VH comprises, consists essentially of, or consists of an amino acid sequence as set forth in SEQ ID NO: 61; and the VL comprises, consists essentially of, or consists of an amino acid sequence as set forth in SEQ ID NO: 63.

Based on the amino acid sequence of heavy chain constant regions of the antibody, an immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (λ) chain or a kappa (κ) chain, based on the amino acid sequence of the light chain. The antibodies described herein can be of any suitable classes or suitable subtypes above.

In some embodiments, which may be combined with other embodiments, the antibody can be of an isotype selected from the group consisting of IgG, IgA, IgM, IgE and IgD. In some embodiments, the antibody can be of a subtype selected from the group consisting of IgG1, IgG2, and IgG3. In an illustrative, but non-limiting, embodiment, the antibody is an IgG1 antibody.

The antibody described herein can be an intact antibody or an antigen binding fragment thereof. The antigen binding fragment can be any suitable fragment(s) of the antibody that retain the ability to specifically bind to CD14. Illustrative, but non-limiting, examples of antigen binding fragments can include, but are not limited to, a Fab fragment; a F(ab′)2 fragment; a Fab′ fragment; a Fd fragment; a Fd′ fragment; a Fv fragment; a scFv fragment; a dAb fragment; an isolated complementarity determining region (CDR); a nanobody; a linear antibody comprising a pair of tandem Fd segments (VH-CH1-VH-CH1), or a modified version of any of the foregoing fragments, which retains antigen binding activity.

In some embodiments, which may be combined with other embodiments, an antigen binding fragment can be selected from the group consisting of Fab, Fab′, F(ab′)2, Fv, scFv, and ds-scFv. In an illustrative, but non-limiting, embodiment, the antigen binding fragment is Fab.

In some embodiments, which may be combined with other embodiments, the antibody can be a monoclonal antibody.

In at least one embodiment, which may be combined with other embodiments, antibodies (or an antigen binding fragment thereof) described herein can comprise a heavy chain constant region (CH1-Hinge-CH2-CH3), also referred to as CH. The heavy chain constant region can comprise, consist essentially of, or consist of an amino acid sequence as set forth in SEQ ID NO: 130. In some embodiments, the heavy chain constant region can comprise or consist of an amino acid sequence having at least 80%, at least 85%, at least 90%, at least 95%, at least 98%, at least 99%, or 100% sequence identity to one or more amino acid sequence as set forth in SEQ ID NO: 130.

In at least one embodiment, which may be combined with other embodiments, antibodies (or an antigen binding fragment thereof) described herein can comprise a light chain constant region (CL). The light chain constant region can comprise, consist essentially of, or consist of an amino acid sequence as set forth in SEQ ID NO: 150. In some embodiments, which may be combined with other embodiments, the light chain constant region can comprise or consist of an amino acid sequence having at least 80%, at least 85%, at least 90%, at least 95%, at least 98%, at least 99%, or 100% sequence identity to one or more amino acid sequence as set forth in SEQ ID NO: 150.

In some embodiments, which may be combined with other embodiments, antibodies (or an antigen binding fragment thereof) described herein can comprise or consist of a heavy chain and a light chain. The heavy chain can comprise or consist of an amino acid sequence as set forth in one or more of SEQ ID NOs: 2, 6, 10, 14, 18, 22, 26, 30, 34, 38, 42, 46, 50, 54, 58, or 62. The light chain can comprise or consist of an amino acid sequence as set forth in one or more of SEQ ID NOs: 4, 8, 12, 16, 20, 24, 28, 32, 36, 40, 44, 48, 52, 56, 60, or 64.

In some embodiments, which may be combined with other embodiments, the heavy chain can comprise or consist of an amino acid sequence having at least 80%, at least 85%, at least 90%, at least 95%, at least 98%, at least 99%, or 100% sequence identity to one or more amino acid sequences as set forth in SEQ ID NOs: 2, 6, 10, 14, 18, 22, 26, 30, 34, 38, 42, 46, 50, 54, 58, or 62; the light chain can comprise or consist of an amino acid sequence having at least 80%, at least 85%, at least 90%, at least 95%, at least 98%, at least 99%, or 100% sequence identity to one or more amino acid sequence as set forth in SEQ ID NOs: 4, 8, 12, 16, 20, 24, 28, 32, 36, 40, 44, 48, 52, 56, 60, or 64; or combinations thereof.

The heavy chain can comprise or consist of a functional variant of an amino acid sequence formed by insertion, deletion, substitution, or combinations thereof, of one or more amino acid(s) therein, provided that the functional variant retains the ability of binding to CD14. In some embodiments, which may be combined with other embodiments, the heavy chain comprises or consists of a functional variant of an amino acid sequence as set forth in one or more of SEQ ID NOs: 2, 6, 10, 14, 18, 22, 26, 30, 34, 38, 42, 46, 50, 54, 58, or 62 formed by insertion, deletion, and/or substitution of one or more amino acid(s) therein, provided that the functional variant retains the ability of binding to CD14.

The light chain can comprise or consist of a functional variant of an amino acid sequence formed by insertion, deletion, substitution, or combinations thereof, of one or more amino acid(s) therein, provided that the functional variant retains the ability of binding to CD14. In some embodiments, which may be combined with other embodiments, the light chain comprises or consists of a functional variant of an amino acid sequence as set forth in one or more of SEQ ID NOs: 4, 8, 12, 16, 20, 24, 28, 32, 36, 40, 44, 48, 52, 56, 60, or 64 formed by insertion, deletion, and/or substitution of one or more amino acid(s) therein, provided that the functional variant retains the ability of binding to CD14.

For example, the functional variant of the amino acid sequence as set forth in SEQ ID NO: 2 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 2. This applies to functional variants of other SEQ ID NOs described herein. For example, a functional variant of an amino acid sequence as set forth in one or more of SEQ ID NO: 2, 6, 10, 14, 18, 22, 26, 30, 34, 38, 42, 46, 50, 54, 58, or 62 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to an amino acid sequence as set forth in SEQ ID NO: 2, 6, 10, 14, 18, 22, 26, 30, 34, 38, 42, 46, 50, 54, 58, or 62, respectively.

As another example, the functional variant of the amino acid sequence as set forth in SEQ ID NO: 4 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 4. This applies to functional variants of other SEQ ID NOs described herein. For example, a functional variant of SEQ ID NO: 4, 8, 12, 16, 20, 24, 28, 32, 36, 40, 44, 48, 52, 56, 60, or 64 can comprise or consist of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 4, 8, 12, 16, 20, 24, 28, 32, 36, 40, 44, 48, 52, 56, 60, or 64, respectively.

In some embodiments, which may be combined with other embodiments, the number of inserted, deleted, and/or substituted amino acids is preferably can be no more than 40% of the total number of amino acids in the parent amino acid sequence, such as no more than 35%, such as 1-33%, such as 5-30%, such as 10-25%, such as 15-20%. For example, the number of inserted, deleted, and/or substituted amino acids can be 1-50 amino acids, such as 1-20 amino acids, such as 1-10 amino acids, such as 1-7 amino acids, such as 1-5 amino acids, such as 1-2 amino acids. In at least one embodiment, the number of inserted, deleted, and/or substituted amino acids can be 1, 2, 3, 4, 5, 6, or 7 amino acids.

In some embodiments, which may be combined with other embodiments, the insertion, deletion, and/or substitution can be performed at framework (FR) regions, for example, at FR1, FR2, FR3, FR4, or combinations thereof; and/or constant regions, for example, CL, CH1, CH2, CH3, or combinations thereof. In some embodiments, the insertion, deletion, and/or substitution can be performed at a CH region.

In some embodiments, which may be combined with other embodiments, the substitution of one or more amino acid(s) can be conservative substitution of one or more amino acid(s). Examples of conservative substitutions are as described above.

In some embodiments, the heavy chain comprises, consists essentially of, or consists of an amino acid sequence as set forth in SEQ ID NO: 2; and the light chain comprises, consists essentially of, or consists of an amino acid sequence as set forth in SEQ ID NO: 4.

In some embodiments, the heavy chain comprises, consists essentially of, or consists of an amino acid sequence as set forth in SEQ ID NO: 6; and the light chain comprises, consists essentially of, or consists of an amino acid sequence as set forth in SEQ ID NO: 8.

In some embodiments, the heavy chain comprises, consists essentially of, or consists of an amino acid sequence as set forth in SEQ ID NO: 10; and the light chain comprises, consists essentially of, or consists of an amino acid sequence as set forth in SEQ ID NO: 12.

In some embodiments, the heavy chain comprises, consists essentially of, or consists of an amino acid sequence as set forth in SEQ ID NO: 14; and the light chain comprises, consists essentially of, or consists of an amino acid sequence as set forth in SEQ ID NO: 16.

In some embodiments, the heavy chain comprises, consists essentially of, or consists of an amino acid sequence as set forth in SEQ ID NO: 18; and the light chain comprises, consists essentially of, or consists of an amino acid sequence as set forth in SEQ ID NO: 20.

In some embodiments, the heavy chain comprises, consists essentially of, or consists of an amino acid sequence as set forth in SEQ ID NO: 22; and the light chain comprises, consists essentially of, or consists of an amino acid sequence as set forth in SEQ ID NO: 24.

In some embodiments, the heavy chain comprises, consists essentially of, or consists of an amino acid sequence as set forth in SEQ ID NO: 26; and the light chain comprises, consists essentially of, or consists of an amino acid sequence as set forth in SEQ ID NO: 28.

In some embodiments, the heavy chain comprises, consists essentially of, or consists of an amino acid sequence as set forth in SEQ ID NO: 30; and the light chain comprises, consists essentially of, or consists of an amino acid sequence as set forth in SEQ ID NO: 32.

In some embodiments, the heavy chain comprises, consists essentially of, or consists of an amino acid sequence as set forth in SEQ ID NO: 34; and the light chain comprises, consists essentially of, or consists of an amino acid sequence as set forth in SEQ ID NO: 36.

In some embodiments, the heavy chain comprises, consists essentially of, or consists of an amino acid sequence as set forth in SEQ ID NO: 38; and the light chain comprises, consists essentially of, or consists of an amino acid sequence as set forth in SEQ ID NO: 40.

In some embodiments, the heavy chain comprises, consists essentially of, or consists of an amino acid sequence as set forth in SEQ ID NO: 42; and the light chain comprises, consists essentially of, or consists of an amino acid sequence as set forth in SEQ ID NO: 44.

In some embodiments, the heavy chain comprises, consists essentially of, or consists of an amino acid sequence as set forth in SEQ ID NO: 46; and the light chain comprises, consists essentially of, or consists of an amino acid sequence as set forth in SEQ ID NO: 48.

In some embodiments, the heavy chain comprises, consists essentially of, or consists of an amino acid sequence as set forth in SEQ ID NO: 50; and the light chain comprises, consists essentially of, or consists of an amino acid sequence as set forth in SEQ ID NO: 52.

In some embodiments, the heavy chain comprises, consists essentially of, or consists of an amino acid sequence as set forth in SEQ ID NO: 54; and the light chain comprises, consists essentially of, or consists of an amino acid sequence as set forth in SEQ ID NO: 56.

In some embodiments, the heavy chain comprises, consists essentially of, or consists of an amino acid sequence as set forth in SEQ ID NO: 58; and the light chain comprises, consists essentially of, or consists of an amino acid sequence as set forth in SEQ ID NO: 60.

In some embodiments, the heavy chain comprises, consists essentially of, or consists of an amino acid sequence as set forth in SEQ ID NO: 62; and the light chain comprises, consists essentially of, or consists of an amino acid sequence as set forth in SEQ ID NO: 64.

In some embodiments, which may be combined with other embodiments, an antibody described herein optionally includes an Fc region. The Fc region can be of any suitable isotype, including, but not limited to, IgG1, IgG2, IgG3, or IgG4, and can comprise one or more mutations or modifications. In at least one embodiment, the Fc region can be of IgG1 isotype or derived therefrom, optionally with one or more mutations or modifications. In at least one embodiment, the Fc region can be human IgG1 Fc.

The Fc region may be effector-function-deficient. For example, the Fc region may be of an IgG1 isotype, or a non-IgG1 type, for example, IgG2, IgG3 or IgG4, which has been mutated such that the ability to mediate effector functions, such as antibody-dependent cellular cytotoxicity (ADCC), has been reduced or even eliminated. Such mutations are, for example, described in W. F. Dall'Acqua et al., J. Immunol., 2006, 177(2), 1129-38; and M. Hezarch et al., J. Virol., 2001, 75(24), 12161-8.

In at least one embodiment, which may be combined with other embodiments, the Fc region comprises a mutation removing the acceptor site for Asn-linked glycosylation or is otherwise manipulated to change the glycosylation properties. For example, in an Fc region of IgG1, an N297A or N297Q mutation can be used to remove an Asn-linked glycosylation site. In some embodiments, which may be combined with other embodiments, the Fc region comprises an IgG1 sequence with an N297A mutation and/or an N297Q mutation. In at least one embodiment, which may be combined with other embodiments, the Fc region can be glyco-engineered to decrease fucose and increase ADCC, for example, by addition of compounds to the culture media during antibody production as described in, for example, U.S. 2009/0317869, or as described in P. H. C. van Berkel et al., Biotechnol. Bioeng., 2010, 105(2), 350-57, or by using FUT8 knockout cells, for example, as described in N. Yamane-Ohnuki et al., Biotechnol. Bioeng., 2004, 87(5), 614-22. ADCC may alternatively be optimized using the method described by P. Umana et al., Nature Biotechnol., 1999, 17(2), 176-80. In at least one embodiment, which may be combined with other embodiments, the Fc region has been engineered to enhance complement activation, for example, as described in A. Natsume et al., Cancer Sci., 2009, 100(12), 2411-2418.

In some embodiments, which may be combined with other embodiments, the Fc region can comprise modifications or mutations that can inhibit Fc homodimerization. In some embodiments, which may be combined with other embodiments, the Fc region comprises a variant of a human IgG1 Fc wild type sequence. The variant can comprise amino acid substitutions at positions T366 and Y407 of human IgG1 (Kabat numbering). For example, T366 can be substituted with L (Leucine). For example, Y407 can be substituted with I (Isoleucine), F (Phenylalanine), L (Leucine), M (Methionine), H (Histidine), K (Lysine), S (Serine), Q (Glutamine), T (Threonine), W (Tryptophan), A (Alanine), G (Glycine) or N (Asparagine). As another example, Y407 can be substituted with H. In at least one embodiment, which may be combined with other embodiments, T366 can be substituted with L, and Y407 can be substituted with H.

In some embodiments, which may be combined with other embodiments, the Fc region can be a monomeric human IgG1 Fc (for example, mFc such as mFc7.2) as described in WO 2018/144784, which is incorporated herein by reference in its entirety.

In some embodiments, which may be combined with other embodiments, the antibody comprises a first polypeptide chain comprising the VL of the antigen binding region and optionally a Fc region; and a second polypeptide chain comprising the VH of the antigen binding region and optionally a Fc region. The Fc region can be those as describe above.

In at least one embodiment, which may be combined with other embodiments, the first polypeptide chain can further comprise a light chain constant region (CL). In some embodiments, which may be combined with other embodiments, the first polypeptide chain can comprise a monomeric human IgG1 Fc (for example, mFc such as mFc7.2) as described herein. In some embodiments, which may be combined with other embodiments, the first polypeptide chain can comprise, from N-terminal to C-terminal: the VL of the antigen binding region, CL, and mFc (such as mFc7.2).

In some embodiments, which may be combined with other embodiments, the second polypeptide chain can further comprise a heavy chain constant region (CH), for example, CH1. In some embodiments, which may be combined with other embodiments, the first polypeptide chain comprises a monomeric human IgG1 Fc (for example, mFc such as mFc7.2) as described herein. In some embodiments, which may be combined with other embodiments, the second polypeptide chain comprises, from N-terminal to C-terminal: the VH of the antigen binding region, CH1, and mFc (such as mFc7.2).

In some embodiments, which may be combined with other embodiments, antibodies described herein can comprise: (i) two light chain regions with each comprising two domains (VL and CL); (ii) two heavy chain regions with each comprising a variable domain (VH) and a constant region (for example, CH1, CH2, CH3, etc.).

In some embodiments, which may be combined with other embodiments, antibodies described herein can comprise two antigen binding fragments (for example, Fab) each containing a VL, a VH, a CL, and a CH1 domain, as well as a crystallizable fragment (Fc) containing CH2 and CH3. In between the antigen binding fragments and the crystallizable fragment is a hinge region.

Antibodies and fragments thereof described herein can be modified for use by the addition, at the amino- and/or carboxyl-terminal ends, of a blocking agent. Such blocking agents can include, for example, additional related or unrelated peptide sequences that can be attached to the amino and/or carboxyl terminal residues of the peptide to be administered. For example, one or more non-naturally occurring amino acids, such as D-alanine, can be added to the termini. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Additionally, the peptide terminus can be modified, for example, by acetylation of the N-terminus and/or amidation of the C-terminus. Likewise, the peptides can be covalently or non-covalently coupled to pharmaceutically acceptable “carrier” proteins prior to use.

Antibodies (or fragment thereof) described herein can include a signal peptide. The signal peptide may include SEQ ID NO: 129 (MHSSALLCCLVLLTGVRA).

Embodiments of the present disclosure also relate to a nucleic acid molecule comprising a nucleotide sequence encoding the antibody or the antigen binding fragment thereof described herein. Embodiments described herein also provide a vector comprising a nucleic acid molecule (or nucleotide sequence) described herein.

Any suitable vector can be utilized. In some embodiments, which may be combined with other embodiments, the vector can be a viral vector. In some embodiments, the vector can be a retroviral vector, a DNA vector, an SFG vector, a murine leukemia virus vector, a plasmid, a RNA vector, a papovaviral vector, a vaccinia viral vector, a herpes simplex viral vector, an adenovirus associated vector (AAV), an adenoviral vector, a baculoviral vector, an Epstein Barr viral vector, a lentiviral vector, or suitable combinations thereof.

Illustrative, but non-limiting, vectors include, for example, pBY, pGAR, pBABE-puro, pBABE-neolargeTcDNA, pBABE-hygro-hTERT, pMKO.1 GFP, MSCV-IRES-GFP, pMSCV PIG (Puro IRES GFP empty plasmid), pMSCV-loxp-dsRed-loxp-cGFP-Puro-WPRE, MSCV IRES Luciferase, pMIG, MDH1-PGK-GFP 2.0, TtRMPVIR, pMSCV-IRES-mCherry FP, pRetroX GFP T2A Cre, pRXTN, pLncEXP, and pLXIN-Luc.

A recombinant expression vector can be any suitable recombinant expression vector. Suitable vectors include those that are designed for propagation and expansion, or for expression, or both, such as plasmids and viruses. For example, a vector may be selected from the pET series (Novagen, Madison, Wis.); the pBluescript series (Stratagene, LaJolla, Calif.); the pUC series (Fermentas Life Sciences, Glen Burnie, Md.); the pEX series (Clontech, Palo Alto, Calif.); and/or the pGEX series (Pharmacia Biotech, Uppsala, Sweden). In some embodiments, bacteriophage vectors such as λGT10, λGT11, λZapII (Stratagene), λEMBL4, and/or λNM1149 can be utilized.

Illustrative, but non-limiting, examples of suitable plant expression vectors useful for embodiments described herein can comprise pBY, pBI101, pBI101.2, pBI101.3, pBI121 (available from Clontech), and pBIN19 (available from ATCC). Illustrative, but non-limiting, examples of suitable animal expression vectors useful for embodiments described herein can comprise pcDNA, pEUK-C1, pMAM, and pMAMneo (available from Clontech).

Recombinant expression vectors can be prepared using standard recombinant DNA techniques described in, for example, J. Sambrook et al, “Molecular Cloning: A Laboratory Manual” (3rd ed.), 2001, Cold Spring Harbor Press; F. M. Ausubel et al, eds., “Current Protocols in Molecular Biology”, 1994, Green Publishing Associates and John Wiley & Sons, New York.

Constructs of expression vectors, which can be circular or linear, can be prepared to contain a replication system functional in a prokaryotic or eukaryotic host cell. Replication systems can be derived, for example, from ColE1, 2μ plasmid, λSV40, bovine papilloma virus, among others.

Embodiments of the present disclosure also include host cell comprising a nucleic acid molecule described herein or a vector described herein. Any suitable cell can be used as a host cell for the nucleic acid molecules or the vectors described herein.

In some embodiments, which may be combined with other embodiments, the cell can be a prokaryotic cell, fungal cell, yeast cell, or higher eukaryotic cells such as a mammalian cell. Illustrative, but non-limiting, suitable prokaryotic cells can include eubacteria, such as Gram-negative or Gram-positive organisms, for example, Enterobactehaceae such as Escherichia, for example, E. coli; Enterobacter; Erwinia; Klebsiella; Proteus; Salmonella, for example, Salmonella typhimurium; Serratia, for example, Serratia marcescans and Shigella; Bacilli such as B. subtilis and B. licheniformis; Pseudomonas such as P. aeruginosa; and Streptomyces. In some embodiments, which may be combined with other embodiments, the cell can be a human cell. In at least one embodiment, which may be combined with other embodiments, the cell can be an immune cell. In some embodiments, host cells can include, for example, Chinese hamster ovary (CHO) cells, such as CHO-S cells or CHO-K1 cells, and/or HEK293 cells, such as HEK293A, HEK293T, or HEK293FS cells.

In some embodiments, which may be combined with other embodiments, a method for preparing an antibody (or fragment thereof) of the present disclosure can generally include expressing, in a suitable host cell, host organism, or other suitable expression system, a nucleic acid molecule (or nucleotide sequence) that encodes the antibody (or fragment thereof). The method can further include isolating and/or purifying the antibody (or fragment thereof) obtained.

In another embodiment, which may be combined with other embodiments, a method for preparing an antibody (or fragment thereof) of the present disclosure can include cultivating and/or maintaining a host under conditions effective to express or produce at least one antibody (or fragment thereof). The method can further include isolating and/or purifying the antibody (or fragment thereof) obtained.

The nucleotide sequence that encodes an antibody (or fragment thereof) described herein can be in the form of a genetic construct. In some embodiments, which may be combined with other embodiments, a nucleic acid molecule (or nucleotide sequence) described herein can encode an amino acid sequence(s) as set forth in one or more SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 31, 32, 33, 34, 35, 36, 37, 38, 39, 41, 42, 43, 44, 45, 46, 47, 48, 49, 51, 52, 53, 54, 55, 56, 57, 58, 59, 61, 62, 63, or 64.

Nucleotide sequences described herein can be in the form of single or double stranded DNA or RNA, such as double stranded DNA. For example, the nucleotide sequences can be genomic DNA, cDNA, or synthetic DNA (such as DNA with a codon usage that has been specifically adapted for expression in the intended host cell or host organism). The nucleotide sequence can be in an essentially isolated form.

As described herein, the nucleic acid molecule (or nucleotide sequence) can be in the form of, be present in, and/or be part of a vector, such as for example a plasmid or other vector which may be in an essentially isolated form.

The nucleic acid molecule (or nucleotide sequence) can be prepared or obtained in a manner known, based on the information on the amino acid sequences for the antibodies (or fragments thereof). In some embodiments, which may be combined with other embodiments, analogs can be obtained by subjecting nucleotide sequences encoding antibodies (or domains thereof, for example, VL and/or VH domains) to, for example, site-directed mutagenesis, so at to provide a nucleic acid molecule (or nucleotide sequence) encoding said analog. In some embodiments, which may be combined with other embodiments, and to prepare a nucleic acid molecule (or nucleotide sequence) described herein, several nucleotide sequences such as at least one nucleotide sequence encoding an antibody (or fragment thereof) described herein and for a nucleic acid molecule (or nucleotide sequence) encoding one or more linkers can be linked together in any suitable manner.

Techniques for generating nucleic acid molecules (or nucleotide sequences) of the present disclosure will be clear to those skilled in the art. Suitable techniques can include, for example, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (for example, to create cassettes and/or regions that may easily be digested and/or ligated using suitable restriction enzymes), and/or the introduction of mutations by means of a PCR reaction using one or more “mismatched” primers, using for example a sequence of a naturally occurring form of CXCR2 as a template. These and other techniques will be clear to those skilled in the art, and reference is again made to the standard handbooks, such as Sambrook et al. and Ausubel et al., described herein, as well as in the Examples section.

The nucleic acid molecule (or nucleotide sequence) described herein can also be in the form of, be present in, and/or be part of a genetic construct. Such genetic constructs generally comprise at least one nucleic acid molecule (or nucleotide sequence) of the present disclosure that is optionally linked to one or more elements of genetic constructs, such as one or more suitable regulatory elements (for example, a suitable promoter(s), enhancer(s), terminator(s), etc.) and optionally further elements of genetic constructs referred to herein.

The genetic constructs of the present disclosure may be DNA or RNA, such as double-stranded DNA. Genetic constructs described herein can be in a form suitable for transformation of the intended host cell or host organism, in a form suitable for integration into the genomic DNA of the intended host cell, or in a form suitable for independent replication, maintenance, and/or inheritance in the intended host organism. For example, genetic constructs described herein can be in the form of a vector, such as for example a plasmid, cosmid, YAC, a viral vector, or transposon, among others. The vector can be an expression vector. The expression vector can be a vector that can provide for expression in vitro and/or in vivo (for example, in a suitable host cell, host organism, expression system, or combinations thereof).

In an illustrative, but non-limiting, example, a genetic construct described herein comprises: (i) at least one nucleic acid molecule (or nucleotide sequence) described herein; operably connected to (ii) one or more regulatory elements, such as a promoter and optionally a suitable terminator; and optionally (iii) one or more further elements of genetic constructs.

The terms “operably connected” and “operably linked” have their usual meaning in the art (as further described herein); and in which “further elements” present in the genetic constructs can, for example, be 3′- or 5′-UTR sequence, a leader sequence, a selection marker, expression marker/reporter gene, and/or elements that can facilitate or increase (the efficiency of) transformation or integration. These and other suitable elements for such genetic constructs will be clear to those skilled in the art, and can for instance depend upon the type of construct used, the intended host cell or host organism, the manner in which the nucleotide sequences of the present disclosure of interest are to be expressed (for example, via constitutive, transient or inducible expression), and/or the transformation technique to be used. For example, regulatory sequences, promoters, and terminators known per se for the expression and production of antibodies and antibody fragments (including, but not limited to, (single) domain antibodies and ScFv fragments) may be used in an essentially analogous manner.

“Operably linked” generally means that portions of the genetic construct are in a functional relationship with each other. For example, a promoter is considered “operably linked” to a coding sequence if the promoter is able to initiate or otherwise control/regulate the transcription and/or the expression of a coding sequence (in which said coding sequence should be understood as being under the control of said promotor). Generally, when two nucleotide sequences are operably linked, they will be in the same orientation and usually also in the same reading frame. They will usually also be essentially contiguous, although this may also not be required.

Nucleic acid molecules (or nucleotide sequences) described herein and/or genetic constructs described herein can be utilized to transform a host cell or host organism, for example, for expression and/or production of an antibody (or fragment thereof) described herein. Suitable hosts or host cells will be clear to those skilled in the art, and can for example be any suitable fungal, prokaryotic or eukaryotic cell or cell line or any suitable fungal, prokaryotic or eukaryotic organism.

Nucleic acids, nucleic acid molecules, nucleotide sequences, and genetic constructs described herein can form at least a portion of a recombinant nucleic acid construct.

In some embodiments, which may be combined with other embodiments, a nucleic acid molecule and/or a recombinant nucleic acid construct comprises, consists essentially of, or consists of:
(a) a nucleotide sequence as set forth in one or more of SEQ ID NOs: 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or a complement thereof;
(b) a nucleotide sequence that encodes an antibody (or fragment thereof) comprising an amino acid sequence as set forth in one or more of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 31, 32, 33, 34, 35, 36, 37, 38, 39, 41, 42, 43, 44, 45, 46, 47, 48, 49, 51, 52, 53, 54, 55, 56, 57, 58, 59, 61, 62, 63, 64, or a complement thereof;
(c) a nucleotide sequence having at least 70%, at least 80%, at least 90%, at least 95%, at least 98%, or at least 99% identity to the nucleotide sequence of any one of (a) or (b);
(d) a nucleotide sequence which anneals under stringent hybridization conditions to the nucleotide sequence of any one of (a) to (c), or a complement thereof;
(e) a nucleotide sequence that differs from the nucleotide sequences of any one of (a) to (d) above due to the degeneracy of the genetic code;
(f) a functional fragment of a nucleotide sequence of any one of (a) to (e); or
(g) any combination of the nucleotide sequences of (a)-(f).

In some embodiments, which may be combined with other embodiments, nucleic acid molecules or nucleotide sequences described herein can be a complement (which can be either a full complement or a partial complement) of a nucleic acid molecule or nucleotide sequence of the present disclosure. Two nucleotide sequences can be considered to be substantially complementary when the two sequences hybridize to each other under stringent conditions. In some embodiments, two nucleotide sequences considered to be substantially complementary hybridize to each other under highly stringent conditions. Stringent conditions refers to a melting temperature above 65° C., indicating the strength of the hybridization.

In some embodiments, which may be combined with other embodiments, nucleic acid molecules (or nucleotide sequences) of the present disclosure can be operatively linked and/or associated with a variety of promoters for expression in cells. Thus, nucleic acid molecules and/or a recombinant nucleic acid constructs described herein can further include one or more promoters operably linked to one or more nucleotide sequences.

The recombinant nucleic acid construct can be an expression cassette or can be included within an expression cassette. As used herein, “expression cassette” refers to a recombinant nucleic acid construct comprising a nucleotide sequence of interest (for example, one or more of (a)-(g) described above, wherein said nucleotide is nucleotide sequence can be operably associated with at least a control sequence (for example, a promoter). Accordingly, some embodiments of the present disclosure, which may be combined with other embodiments, provide expression cassettes designed to express the nucleotide sequences described herein in a cell.

An expression cassette comprising a nucleotide sequence can be chimeric, meaning that at least one of its components is heterologous with respect to at least one of its other components. An expression cassette can also be one that is naturally occurring but has been obtained in a recombinant form useful for heterologous expression. An expression cassette also can optionally include a transcriptional and/or translational termination region (i.e., termination region) that is functional in the cell in which the nucleotide sequence of interest is to be expressed.

A variety of transcriptional terminators are available for use in expression cassettes and are responsible for the termination of transcription beyond the heterologous nucleotide sequence of interest and correct mRNA polyadenylation. The termination region can be native to the transcriptional initiation region, can be native to the operably linked nucleotide sequence of interest, can be native to the host organism, or can be derived from another source (i.e., foreign or heterologous to the promoter, the nucleotide sequence of interest, the host organism, or any combination thereof). In addition, and in some embodiments, a coding sequence's native transcription terminator can be used.

An expression cassette can include a nucleotide sequence for a selectable marker, which can be used to select a transformed organism and/or cell. As used herein, “selectable marker” refers to a nucleotide sequence that when expressed imparts a distinct phenotype to the transformed organism or cell expressing the marker and thus allows such transformed organisms or cells to be distinguished from those that do not have the marker. Such a nucleotide sequence can encode either a selectable or screenable marker, depending on whether the marker confers a trait that can be selected for by chemical means, such as by using a selective agent (for example, an antibiotic, herbicide, or the like), or on whether the marker is simply a trait that one can identify through observation or testing, such as by screening. Of course, many examples of suitable selectable markers useful in various organisms are known in the art and can be used in the expression cassettes described herein.

In addition to expression cassettes, nucleic acid molecules and nucleotide sequences described herein can be used in connection with vectors. The term “vector” refers to a composition for transferring, delivering or introducing a nucleic acid (or nucleic acid molecules) into a cell. A vector includes a nucleic acid molecule comprising the nucleotide sequence(s) to be transferred, delivered, and/or introduced. Vectors for use in transformation of animals, plants, and other organisms are well known in the art. Illustrative, but non-limiting, examples of general classes of vectors include a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector can transform prokaryotic or eukaryotic host either by integration into the cellular genome or exist extrachromosomally (for example, an autonomous replicating plasmid with an origin of replication). Additionally included are shuttle vectors by which is meant a DNA vehicle capable, naturally or by design, of replication in two different host organisms, which can be selected from prokaryotic and eukaryotic organisms. In some embodiments, which may be combined with other embodiments, the nucleic acid molecule in the vector is under the control of, and operably linked to, an appropriate promoter or other regulatory elements for transcription in a host cell such as a microbial, for example, bacterial, or an animal or a plant cell. The vector can be a bi-functional expression vector which functions in multiple hosts. In the case of genomic DNA, this can contain its own promoter or other regulatory elements and in the case of cDNA this can be under the control of an appropriate promoter or other regulatory elements for expression in the host cell.

Nucleic acid molecules (or nucleotide sequences) described herein and/or genetic constructs described herein can include a restriction site for the enzyme Not1, a restriction site for the enzyme Xba1, or combinations thereof. In some embodiments, which may be combined with other embodiments, the restriction site for the enzyme Not1 comprises GCGGCCGC. In some embodiments, which may be combined with other embodiments, the restriction site for the enzyme Xba1 comprises TCTAGA.

In some embodiments, which may be combined with other embodiments, the nucleic acid molecule (or nucleotide sequences) comprise a promoter sequence operably linked to the polynucleotide. In some embodiments, which may be combined with other embodiments, the nucleic acid molecule is contained within a viral vector described above.

Embodiments of the present disclosure also generally relate to compositions such as pharmaceutical compositions. In some embodiments, which may be combined with other embodiments, pharmaceutical compositions described herein can be used for the treatment of a disease, such as cancer, in a subject. The subject may be a patient such as a mammal.

In some embodiments, which may be combined with other embodiments, the present disclosure provides a pharmaceutical composition comprising (i) an antibody or an antigen binding fragment thereof described herein, and (ii) a pharmaceutically acceptable carrier or excipient.

The pharmaceutical composition can be formulated with pharmaceutically acceptable carriers or diluents as well as any other known adjuvants and excipients in accordance with conventional techniques such as those described in Remington: The Science and Practice of Pharmacy, 19th Edition, Gennaro, Ed., Mack Publishing Co., Easton, Pa., 1995. In at least one embodiment, which may be combined with other embodiments, the pharmaceutical compositions can include a combination of multiple (for example, two or more) isolated antibodies of the present disclosure.

For purposes of the present disclosure, a “pharmaceutically acceptable carrier” includes any suitable solvent, dispersion medium, coating, antibacterial agent, antifungal agent, isotonic delaying agent, and absorption delaying agent, and the like that are physiologically compatible. In some embodiments, which may be combined with other embodiments, the carrier can be suitable for intravenous, intramuscular, subcutaneous, parenteral, spinal or epidermal administration (for example, by injection or infusion). Depending on the route of administration, the active compound, e.g., antibody or antigen binding fragment thereof, can be coated in a material to protect the compound from the action of acids and other natural conditions that may inactivate the compound.

Pharmaceutically acceptable salts can be used. Such salts are those that retain the desired biological activity of the parent compound and does not impart any undesired toxicological effects (see, for example, S. M. Berge, et al., J. Pharm. Sci., 1977, 66, 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, combinations thereof, and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, and procaine, combinations thereof, among others.

In some embodiments, which may be combined with other embodiments, the carrier or excipient for use with the composition described herein can include, but is not limited to, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, surfactant polyoxyethylene-sorbitan monooleate, or combinations thereof.

In some embodiments, which may be combined with other embodiments, the pharmaceutical composition further comprises a second therapeutic agent. In some embodiments, which may be combined with other embodiments, the second therapeutic agent can comprise, or consist of an antibody (or fragment thereof), a chemotherapeutic agent, a small molecule drug, or combinations thereof. The antibody (or fragment thereof) can be different from the antibody (or antigen binding fragments thereof) described herein.

In some embodiments, which may be combined with other embodiments, the second therapeutic agent can be selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, glucocorticoid, and combinations thereof.

In some embodiments, which may be combined with other embodiments, the second therapeutic agent can include, for example, cytotoxic agents, anti-metabolite agents (for example, folate antagonists, purine analogs, pyrimidine analogs, among others), topoisomerase inhibitors (for example, camptothecin derivatives, anthracenedione, anthracyclines, epipodophyllotoxins, quinoline alkaloids, among others), anti-microtubule agents (for example, taxanes, vinca alkaloids, among others), protein synthesis inhibitors (for example, cephalotaxine, camptothecin derivatives, quinoline alkaloids, among others), alkylating agents (for example, alkyl sulfonates, ethylenimines, nitrogen mustards, nitrosoureas, platinum derivatives, triazenes, among others), alkaloids, terpenoids, kinase inhibitors, or combinations thereof.

In some embodiments, which may be combined with other embodiments, the second therapeutic agent can include, for example, immunomodulators, radioactive compounds, enzymes (for example perforin), chemotherapeutic agents (for example, cisplatin), a toxin, or combinations thereof. In some embodiments, which may be combined with other embodiments, the second therapeutic agent can be maytansine, geldanamycin, tubulin inhibitors such as tubulin binding agents (for example, auristatins), or minor groove binding agents such as calicheamicin.

Suitable second therapeutic agents can also include, for example, small molecule cytotoxic agents, such as compounds with the ability to kill mammalian cells having a molecular weight of less than 1,500 Daltons, such as 700 Daltons. Such compounds can also contain toxic metals capable of having a cytotoxic effect. Furthermore, it is contemplated that these small molecule cytotoxic agents also include prodrugs. Prodrugs are compounds that decay or are converted under physiological conditions to release cytotoxic agents. Examples of such agents include cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetrexate glucuronate, auristatin E vincristine and doxorubicin; peptide cytotoxins, which are proteins or fragments thereof with the ability to kill mammalian cells, for example, ricin, diphtheria toxin, pseudomonas bacterial exotoxin A, DNase, and RNase; radio-nuclides, which are unstable isotopes of elements which decay with the concurrent emission of one or more of α or β particles, or γ rays, for example, yttrium-90, indium-111, iodine-131, rhenium-186, bismuth-210, bismuth-213, astatine-213, and actinium-225. Chelating agents may be used to facilitate the association of these radionuclides to the molecules, or multimers thereof. Combinations of the aforementioned are contemplated.

In some embodiments, which may be combined with other embodiments, a composition includes (i) an antibody or an antigen binding fragment thereof described herein, and (ii) a chemical moiety. The chemical moiety can be a detectable moiety, a therapeutic agent, an immune stimulatory molecule, or combinations thereof.

In some embodiments, which may be combined with other embodiments, the detectable moiety can comprise (or be selected from the group consisting of) biotin, streptavidin, an enzyme or catalytically active fragment thereof, a radionuclide, a nanoparticle, a paramagnetic metal ion, or a fluorescent, phosphorescent, chemiluminescent molecule, or combinations thereof. A detectable moiety for diagnostic purposes can include for instance, fluorescent labels, radiolabels, enzymes, nucleic acid probes, contrast reagents, or combinations thereof.

Illustrative, but non-limiting, therapeutic agents are described above.

In some embodiments, which may be combined with other embodiments, the immune stimulatory molecule can be an immune effector molecule which stimulates an immune response. For example, the immune stimulatory molecule can be a cytokine such as IL-2 and IFN-y, a chemokine such as IL-8, platelet factor 4, melanoma growth stimulatory protein, complement activators, viral/bacterial protein domains, viral/bacterial peptides, or combinations thereof.

In some embodiments, which may be combined with other embodiments, antibodies (or antigen binding fragments thereof) described herein may be administered in conjunction with radiotherapy and/or autologous peripheral stem cell or bone marrow transplantation. The antibodies (or antigen binding fragments thereof) can be administered in combination with one or more antibodies selected from anti-CD25 antibodies, anti-CD19 anti-bodies, anti-CD21 antibodies, anti-CD22 antibodies, anti-CD37 antibodies, anti-CD38 anti-bodies, anti-IL6R antibodies, anti-IL8 antibodies, anti-IL15 antibodies, anti-IL15R antibodies, anti-CD4 antibodies, anti-CD11a antibodies (for example, efalizumab), anti-alpha-4/beta-1 integrin (VLA4) antibodies (for example, natalizumab), CTLA4-Ig, or combinations thereof.

Actual dosage levels of the active ingredients in compositions and pharmaceutical compositions of the present disclosure can be varied so as to obtain an amount of the active ingredient which is effective to achieve the desired therapeutic response for a particular patient, composition, and mode of administration, without being toxic to the patient. The selected dosage level can depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” for tumor therapy can be measured by objective tumor responses which can either be complete or partial. A complete response (CR) is defined as no clinical, radiological or other evidence of disease. A partial response (PR) results from a reduction in aggregate tumor size of greater than 50%. Median time to progression is a measure that characterizes the durability of the objective tumor response. A “therapeutically effective dosage” for tumor therapy can also be measured by its ability to stabilize the progression of disease. The ability of a compound to inhibit cancer can be evaluated in an animal model system predictive of efficacy in human tumors. Alternatively, this property of a composition can be evaluated by examining the ability of the compound to inhibit cell growth or apoptosis by in vitro assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

Embodiments of the present disclosure also relate to therapies and combination therapies. The therapy can include administering to a patient (or subject) an amount of an antibody or an antigen binding fragment thereof described herein. The amount can be a single amount or an amount administered over a selected number of days. In some embodiments, which may be combined with other embodiments, the present disclosure provides a combination therapy comprising (i) an antibody or an antigen binding fragment thereof described herein, and (ii) a second therapeutic agent. The second therapeutic agent can be selected from an antibody, a chemotherapeutic agent, and a small molecule drug.

In some embodiments, which may be combined with other embodiments, the present disclosure provides a method of treating a cancer in a subject comprising administering to the subject an effective amount of a combination therapy comprising (i) an antibody or an antigen binding fragment thereof described herein, and (ii) optionally a second therapeutic agent.

Implementations of therapies and combination therapies can include one or more of the following. In some embodiments, which may be combined with other embodiments, the cancer is CD14 positive cancer. In some embodiments, which may be combined with other embodiments, the cancer can be selected from the group consisting of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, and combinations thereof. In some embodiments, which may be combined with other embodiments, the cancer comprises bladder cancer, recurrent bladder cancer, or combinations thereof. In some embodiments, which may be combined with other embodiments, the second therapeutic agent can be selected from an antibody, a chemotherapeutic agent, and a small molecule drug.

In some embodiments, which may be combined with other embodiments, the combination therapy, which may be administered to a subject, comprises a dosing regimen comprising:
(a) administering an antibody or an antigen binding fragment thereof described herein every 3 or 4 days on days 0, 4, 7, 11, 14, 18, 21, 25, and 28 of a first 28-day cycle; and
(b) optionally, administering the second therapeutic agent every 3 or 4 days on days 0, 4, 7, 11, 14, 18, 21, 25, and 28 of a first 28-day cycle.

The antibody or the antigen binding fragment thereof described herein can be administered at an amount of 5 mg/kg to about 40 mg/kg, such as from about 5 mg/kg to about 25 mg/kg, such as from about 5 mg/kg to about 15 mg/kg. These amounts, can be given at each treatment day or be a total amount administered over the 28-day cycle. In some embodiments, which may be combined with other embodiments, the second therapeutic agent can be administered at an amount of 1 mg/kg to about 40 mg/kg, such as from about 1 mg/kg to about to 15 mg/kg, such as from about 1 mg/kg to about 10 mg/kg such as from about 3 mg/kg to about 6 mg/kg. These amounts, can be given at each treatment day or be a total amount administered over the 28-day cycle.

Administration of the antibody or antigen binding fragment thereof and the optional second therapeutic agent can be performed by any suitable method such as intravenously, such as an intravenous (IV) infusion.

Embodiments described herein, which may be combined with other embodiments, can be utilized for a variety of applications and uses. One application includes their use in a method of treating cancer.

In some embodiments, which may be combined with other embodiments, a method of treating a cancer in a subject comprises administering to the subject an effective amount of an antibody or an antigen binding fragment thereof described herein, a pharmaceutical composition described herein, or combinations thereof.

In some embodiments of the method described herein, which may be combined with other embodiments, the cancer is a CD14 positive cancer. The cancer can be selected from the group consisting of bladder cancer, recurrent bladder cancer, leukemia, kidney cancer (e.g. renal cell carcinoma), lung cancer, gastric cancer, ovarian cancer, breast cancer, mastocytosis, pancreatic cancer, colon cancer, colorectal cancer, bile duct cancer, liver cancer, Wilms tumor, esophageal cancer, bladder cancer, prostate cancer, uterine cancer, cervical cancer, brain cancer, gastric cancer, cholangiocarcinoma, chondrosarcoma, thyroid cancer, skin cancer, glioma, neuroblastoma, lymphoma, myeloma, and combinations thereof.

In some embodiments, which may be combined with other embodiments, the cancer comprises bladder cancer, recurrent bladder cancer, or combinations thereof. The cancer can be selected from the group consisting of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, and combinations thereof.

In some embodiments, which may be combined with other embodiments, dosage administered to a subject may vary with the embodiment, the medicament employed, the method of administration, and the site and subject being treated. A dose should be sufficient to provide a therapeutic response. A clinician may determine the effective amount to be administered to a human or other subject in order to treat a medical condition. The precise amount to be therapeutically effective may depend upon various factors, for example, such as the activity of the antibody, and the route of administration.

A dose of the antibodies (or antigen binding fragment thereof) and/or compositions described herein may be administered to a mammal at one time or in a series of sub-doses administered over a suitable period of time, for example, on a daily, once every 2 days, once every 3 days, once every 4 days, once every five days, once every six days, semi-weekly, weekly, bi-weekly, semi-monthly, bi-monthly, semi-annual, or annual basis, as needed. A dosage unit comprising an effective amount of antibodies (or antigen binding fragment thereof) and/or compositions described herein can be administered in a single daily dose, or the total daily dosage may be administered in two, three, four, or more divided doses administered daily, as needed.

A suitable means of administration can be selected by a medical practitioner. Routes of administration can include parenteral, for example, administration by injection, transnasal administration, transpulmonary administration, or transcutaneous administration. Administration can be systemic or local by intravenous injection, intramuscular injection, intraperitoneal injection, subcutaneous injection. In some embodiments, which may be combined with other embodiments, the antibodies (or antigen binding fragment thereof) and/or compositions described herein are selected for parenteral delivery, for inhalation, or for delivery through the digestive tract, such as orally.

Dose and method of administration can vary depending on the weight, age, condition, and the like of the subject, and can be suitably selected.

In some embodiments, which may be combined with other embodiments, the method can further include administering to the subject a second therapeutic agent. In some embodiments, which may be combined with other embodiments, an antibody (or antigen binding fragment thereof) described herein or a composition described herein can be administered prior to, substantially simultaneously with, or after the administration of the second therapeutic agent.

The second therapeutic agent can be one or more of those second therapeutic agents described above, for example, those antibodies, chemotherapeutic agents, and small molecule drugs described above. In some embodiments, which may be combined with other embodiments, the second therapeutic agent is a chemotherapeutic agent. Such chemotherapeutic agents can include, for example, alkylating agents (for example, alkyl sulfonates, ethylenimines, nitrogen mustards, nitrosoureas, platinum derivatives, triazenes, etc.), cytotoxic agents, anti-metabolite agents (for example, folate antagonists, purine analogs, pyrimidine analogs, etc.), topoisomerase inhibitors (for example, camptothecin derivatives, anthracenedione, anthracyclines, epipodophyllotoxins, quinoline alkaloids, etc.), anti-microtubule agents (for example, taxanes, vinca alkaloids), protein synthesis inhibitors (for example, cephalotaxine, camptothecin derivatives, quinoline alkaloids), alkaloids, terpenoids, kinase inhibitors, or combinations thereof.

In at least one embodiment, which may be combined with other embodiments, the second therapeutic agent comprises or consists of an alkylating agent. In some embodiments, which may be combined with other embodiments, the second therapeutic agent comprises or consists of cisplatin.

In some embodiments, which may be combined with other embodiments, the present disclosure provides a pharmaceutical pack or kit comprising one or more containers filled with one or more of the ingredients of the pharmaceutical compositions described herein, such as an antibody or an antigen binding fragment thereof described herein. Optionally, associated with such container(s) can be a notice in the form prescribed by a governmental agency regulating the manufacture, use or sale of pharmaceuticals or biological products, which notice reflects approval by the agency of manufacture, use or sale for human administration.

In a specific but non-limiting embodiment, which may be combined with other embodiments, the kit comprises a first container containing an antibody or an antigen binding fragment thereof described herein. In a specific but non-limiting embodiment, which may be combined with other embodiments, the kit comprises a first container that is a vial containing an antibodies or an antigen binding fragment thereof as a lyophilized sterile powder under vacuum, and the kit further comprises a second container comprising a pharmaceutically acceptable fluid.

In some embodiments, which may be combined with other embodiments, the kit includes an injection device containing an antibody or an antigen binding fragment thereof described herein. In a specific but non-limiting embodiment, which may be combined with other embodiments, the injection device comprises the antibody in sterile solution. In a specific but non-limiting embodiment, the injection device is a syringe.

In yet another embodiment, which may be combined with other embodiments, the present disclosure provides use of an antibody (or an antigen binding fragment thereof) described herein or a pharmaceutical composition described herein in the manufacture of a medicament for treating a cancer in a subject. In some embodiments, the cancer is a CD14 positive cancer.

In yet another embodiment, which may be combined with other embodiments, the present disclosure provides an antibody (or the antigen binding fragment thereof) described herein or the pharmaceutical composition described herein for use in treating a cancer in a subject. In some embodiments, which may be combined with other embodiments, the cancer is a CD14 positive cancer. The CD14 positive cancer may be selected from the group consisting of bladder cancer, recurrent bladder cancer, leukemia, kidney cancer, lung cancer, gastric cancer, ovarian cancer, breast cancer, mastocytosis, pancreatic cancer, colon cancer, colorectal cancer, bile duct cancer, liver cancer, Wilms tumor, and combinations thereof. In some examples of the use described herein, the cancer can be bladder cancer, recurrent bladder cancer, or combinations thereof.

The following examples are put forth so as to provide those of ordinary skill in the art with a complete disclosure and description of how to make and use aspects of the present disclosure, and are not intended to limit the scope of embodiments of the present disclosure. Efforts have been made to ensure accuracy with respect to numbers used (for example, amounts, dimensions, etc.) but some experimental errors and deviations should be accounted for.

### EXAMPLES

### Example 1. CD14 is a Marker of Recurrent Bladder Cancer

### 1.1. Human Tumor Sample Collection

Tissue samples from subjects (patients) with bladder cancer who underwent laparoscopic radical cystectomy or partial cystectomy were selected, and full-layer bladder tumor tissues were collected. The tissues were cleaned with sterile normal saline immediately after surgery and stored in precooled MACS tissue storage solution at 4° C. for immunohistochemistry experiments.

### 1.2. CD14 Immunohistochemistry (IHC) Analysis

Paraffin section immunohistochemistry was used to demonstrate the presence and location of proteins in tissue sections. For these analyses, the reagents included: anhydrous ethanol, xylene, hydrogen peroxide (catalog no. 10011218), hydrochloric acid, and ammonia, each available from Sinopharm Chemical Reagent Co., Ltd; phosphate buffered saline (PBS) buffer solution (catalog no. G0002) available from Servicebio; and IHC reagent kit DAB chromogen available from DAKO (catalog no. K5007). Other reagents are provided in the protocol. For the analyses, the following equipment included: For these analyses, the experimental equipment included a dehydrator (model no. JJ-12J), embedding machine (model no. JB-P5), and frozen section machine (model no. JB-L5), each available from Wuhan Junjie Electronics Co., Ltd; slides (model no. 80312-3181) and coverslips (model no. 10212432C), each available from Jiangsu Shitai Experimental Equipment Co., Ltd; vortex mixer (model no. MX-F), and palm centrifuge (model no. D1008E) are available from Servicebio. The pathological microtome is available from Leica Instruments Shanghai Co., Ltd (model no. RM2016). Tissue spreading machine is available from Jinhua City, Zhejiang Province Kodi Instrument Equipment Co., Ltd (model no. KD-P). Oven is available from Shanghai Huitai Instrument Manufacturing Co., Ltd (model no. DHG-9140A). Microwave oven is available from Galanz Microwave Oven Electrical Appliance Co., Ltd (model no. P70D20TL-P4). Pipettes are available from Dragon (model no. KE0003087/KA0056573). Immunohistochemical pen is available from Gene tech (model no. GT1001). Other equipment is provided in the protocol.

The paraffin section immunohistochemistry included the following protocol:

Fixation: Fixation was performed using on poly-L-lysine-coated slides, using 4% paraformaldehyde as a fixative. The fixation time was 24 hours. After fixation, the tissue blocks were embedded in paraffin, then cut in a microtome to the desired thickness (approximately 5 microns) and affixed onto the slide. Once mounted, the slides were dried to remove water that may be trapped under the tissue sections.

Deparaffinization of paraffin sections to water: Tissue sections were sequentially placed in xylene I for 15 minutes, xylene II for 15 minutes, xylene III for 15 minutes, absolute ethanol I for 5 minutes, absolute ethanol II for 5 minutes, 85% alcohol for 5 minutes, 75% alcohol for 5 minutes, and then washed with distilled water.

Antigen retrieval: The tissue sections were placed in a pressure cooker containing citrate antigen retrieval buffer (pH 6.0) (Servicebio, catalog no. G1202). First, the retrieval solution was brought to a boil, then tissue sections were immersed in the retrieval solution, the pressure cooker was covered, and timed for 3 minutes after the steam started. After natural cooling, the slides were placed in phosphate buffered saline (PBS, pH 7.4) and shaken on a decolorizing shaker (Servicebio, model no. TSY-B) to wash 3 times, each time for 5 minutes.

Blocking endogenous peroxidase: The tissue sections were immersed in a 3% hydrogen peroxide solution (hydrogen peroxide:pure water=1:9) and incubated in the dark at room temperature for 25 minutes. The slides were then placed in PBS (pH 7.4) and shaken on the decolorizing shaker to wash 3 times, each time for 5 minutes.

Serum blocking: Bovine serum albumin (BSA, 3%, Servicebio, catalog no. G5001) was applied to evenly cover the tissue within the immunohistochemistry circle, and blocked at room temperature for 30 minutes.

Add primary antibody: The blocking solution was gently shaken off, the primary antibody (diluted in PBS) was applied to the sections, and then incubated overnight at 4° C. in a wet box. (A small amount of water was added to the wet box to prevent the antibody from evaporating). The primary antibody was CD14 Rabbit Polyclonal, available from Proteintech (catalog no. 17000-1-AP) (Dilution ratio: 1:1500).

Add secondary antibody: The slides were placed in PBS (pH 7.4) and shaken on the decolorizing shaker to wash 3 times, each time for 5 minutes. After slightly shaking dry, the secondary antibody (horseradish peroxidase (HRP) labeled) was applied within the circle to cover the tissue, and then incubated at room temperature for 50 minutes. The secondary antibody was HRP-Goat Anti-Rabbit, available from Servicebio (catalog no. GB23303) (Dilution ratio: 1:200).

3,3′-Diaminobenzidine (DAB) staining: The slides were placed in PBS (pH 7.4) and shaken on the decolorizing shaker to wash 3 times, each time for 5 minutes. After slightly shaking dry, a freshly prepared DAB staining solution was applied within the circle, the staining time was controlled under a microscope, the positive expression was brown-yellow, and rinsed the sections with tap water to stop the color development.

Re-stain the cell nucleus: The slide was re-stained with Harris hematoxylin (Servicebio catalog no. G1004) for about 3 minutes, washed with tap water, differentiated with 1% hydrochloric acid alcohol for a few seconds, rinsed with tap water, blued with ammonia water, and rinsed with running water.

Dehydration and sealing: The sections were sequentially placed in 75% alcohol for 5 minutes, 85% alcohol for 5 minutes, absolute ethanol I for 5 minutes, absolute ethanol II for 5 minutes, and xylene I for 5 minutes for dehydration and transparency. The sections were removed from the xylene and were allowed to dry slightly, then sealed with neutral gum (Servicebio, catalog no. G1403).

Microscopic inspection, image acquisition, and analysis were performed. Microscope and image acquisition were performed using a microscope (CIC, model no. XSP-C204, Chongqing, China).

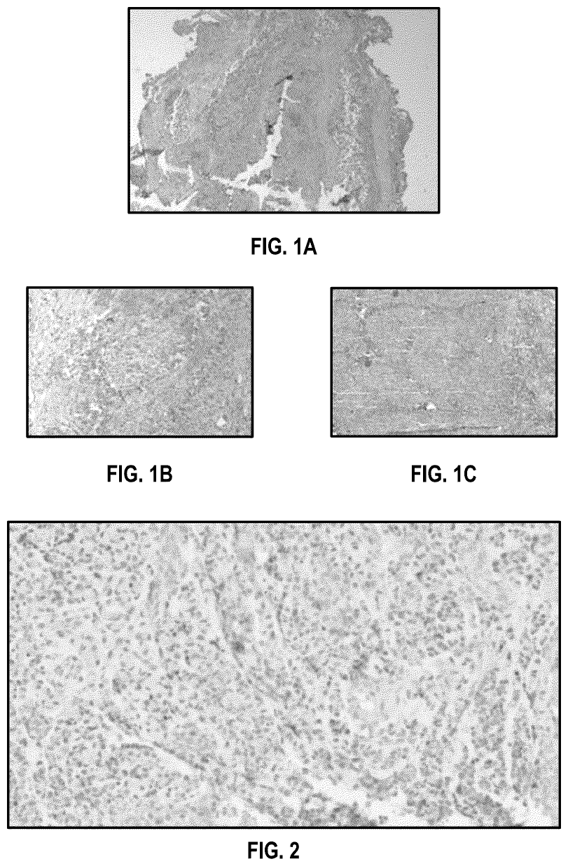

FIGS. 1A-1C show example pathological images illustrating instances of recurrent bladder cancer tumors that exhibit a high expression level of CD14. The sample used was from an example patient with recurrent bladder cancer. Overall, the images indicate strong CD14 positive expression in recurrent bladder cancer cells.

202 pathological staining samples from advanced recurrent malignant bladder cancer cases were tested. Fisher's exact test was utilized to show the difference of CD14 protein expression in recurrent and primary (new) bladder cancer patients. Selected results are shown in Table 1.

TABLE 1

| | A− | A+ | Total |
|---|---|---|---|
| Recurrent | 60 | 142 | 202 |
| New | 40 | 3 | 43 |
| Total | 100 | 145 | 245 |

In an examination of the 202 pathological staining samples from advanced recurrent malignant bladder cancer cases, it was established that 142 instances exhibited overexpression of CD14 in the cancer cells (greater than 5%). Conversely, among 43 primary (novel) cases, a high level of CD14 positive cancers were only observed in 3 instances. Based on Fisher's exact test, the tow-tailed P value was determined to be less than 0.0001. The association between rows (groups) and columns (outcomes) is considered to be statistically significant. Overall, Fisher's exact test underscored a significant disparity between the two groups, with a p-value of less than 0.0001.

### Example 2. Patient-Derived Tumor Xenograft (PDX) Establishment

### 2.1. Tumor Xenograft Establishment on Mouse

Primary tumors from patients can grow in immunodeficient mice and maintain a relatively complete tumor microenvironment. The morphology, structure, and molecular signaling of the transplanted tumors in immunodeficient mice remain unchanged, and their gene expression profiles, and mutation patterns are consistent with the primary tumors in patients. This allows for effective evaluation of drug effects and mechanisms of action in vivo.

After removing the primary tumor tissue from the patient, the tissue was temporarily placed in a complete culture medium containing RPMI1640 (Gibco Roswell Park Memorial Institute 1640 Media)+PS (penicillin and streptomycin)+10% FBS (Fetal bovine serum). The tissue was then transported to a sterile operating room within an SPF-grade (Specific Pathogen Free) animal facility. The tumor tissue was cut to an appropriate size (5 millimeters (mm) in diameter). Severely immunodeficient NSG (NOD scid gamma mouse) mice were anesthetized using an isoflurane respirator. The tumor was transplanted subcutaneously into the right rib area through surgery. When the tumor volume reached 1 cm³, it was passaged following the same procedure, and after expanding for three generations, the tumor became stable for use in formal experiments. During the experiment, passaging and expansion were performed as needed. When the tumor volume reached 100 mm³, the mice were divided into groups according to the experimental requirements and treated with drugs.

### 2.2. Tumor Xenograft CD14+ Immune Staining

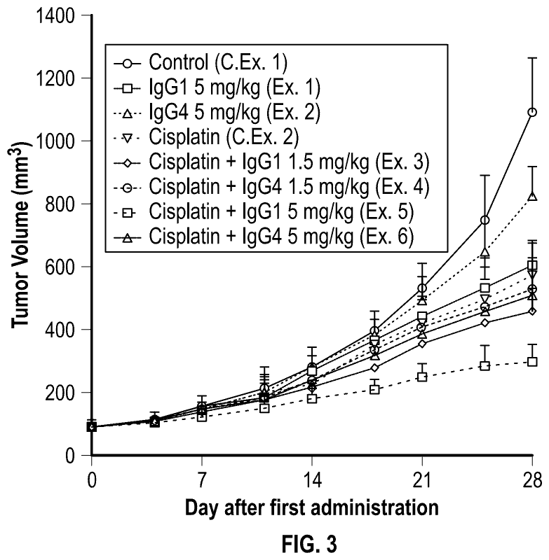

The immunostaining methods employed are analogous to those used in human studies. FIG. 2 is an image showing immunohistochemistry staining of CD14 expression on patient-derived xenograft (PDX) slides. The results indicated a high expression of the CD14 protein in the patient-derived xenograft PDX, demonstrating the successful establishment of the PDX model.

### 2.3. Study Approval

Protocols were approved by the Ethical Committee of Xuzhou Central Hospital (EC. XZXY-LI-20200708-024). Participants were provided written informed consent.

### Example 3. Design of IgG1

IgG1 and IgG4 are two subclasses of Immunoglobulin G (IgG), which is an abundant antibody isotype in human serum. Each subclass has distinct properties and functions. The IgG1 subclass makes up about 60-70% of the total IgG in human serum. IgG1 is effective at fixing complement, a process that enhances the ability of antibodies to clear pathogens or marked cells. The IgG1 subclass also has a high affinity for Fc gamma receptors (FcγRs) on immune cells, such as macrophages and natural killer (NK) cells. This high affinity allows IgG1 to effectively trigger antibody-dependent cellular cytotoxicity (ADCC) and phagocytosis. The IgG4 subclass accounts for about 4-7% of the total IgG in human serum. IgG4 has a structural feature that allows it to exchange Fab arms with other IgG4 molecules, resulting in bispecific antibodies with different antigen-binding specificities. This property can reduce the ability of IgG4 to form large immune complexes, which are typically associated with inflammatory responses. IgG4 has a weaker affinity for FcγRs and a limited capacity to fix complement compared to IgG1, making it less potent in triggering immune responses like ADCC and phagocytosis.

The first modified IgG1 antibody sequence (D6DD3D001) is described further below in Table 5-1. The heavy chain constant region (CH1-Hinge-CH2-CH3) sequences (also referred to as CH sequences) are identical in IgG1 and the subsequently designed antibodies. The heavy chain constant region (CH) of the first modified IgG1 antibody sequence (D6DD3D001) has an amino acid sequence as set forth in SEQ ID NO: 130, and is shown in Table 2.

The sequences of D6DD3D001-D6DD3D016 in the examples are according to the Kabat numbering system.

TABLE 2
D6DD3D001

| | |
|---|---|
| CH | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK (SEQ |
| | ID NO: 130) |

The light chain constant region (CL) sequences are identical in the IgG1 antibody (D6DD3D001) and the subsequently designed antibodies (D6DD3D002-D6DD3D016). The light chain constant region (CL) has an amino acid sequence as set forth in SEQ ID NO: 150, and is shown in Table 3.

TABLE 3
D6DD3D001

| | |
|---|---|
| CL | RTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGL |
| | SSPVTKSFNRGEC (SEQ ID NO: 150) |

### Example 4. Humanized Antibody Design

In clinical applications, murine antibodies have strong immunogenicity and can cause human anti-mouse antibody (HAMA) responses. This leads to a shortened half-life of the murine antibodies, weakened therapeutic effect, and therefore murine antibodies cannot be directly applied in clinical therapy. Humanization design can minimize the immunogenicity of the antibodies. Antibody humanization can follow two principles: one is to reduce or eliminate the immunogenicity of the antibody, and the other is to maintain or enhance the affinity specificity of the antibody. Following these two principles, the murine antibodies were humanized. For the light and heavy chains, three to six candidate humanization sequences were provided, respectively. These humanization candidate sequences were paired and grouped, and each group was transfected and expressed in cells to produce humanized antibodies. Then, the affinity was tested on the Biacore platform to screen out humanized antibodies that both have the affinity of the original murine antibody and have reduced immunogenicity.

### 4.1. Design Method

### 4.1.1. Determination of CDR Region

The antibody variable region (VH or VL, including the part composed of VDJ genes (variable, diversity, and joining)) is usually divided into the framework region (framework) and the complementarity determining region (CDR). The humanization design used an antibody structure simulation and performance analysis platform to predict the structure of the antibody through homology modeling of the antibody structure and analyze the entire CDR structure. Different CDR definition systems may result in variations in the determined CDR regions. Kabat numbering system was used to determine the amino acid sequences of heavy chain variable region (VH), light chain variable region (VL), the whole heavy chain (heavy chain), the whole light chain (light chain) of the Atibuclimab antibody. Table 4-1 shows the amino acid sequences of heavy chain variable region (VH), light chain variable region (VL), the whole heavy chain (heavy chain), the whole light chain (light chain), CDR sequences and the high-risk post-translational modification (PTM) site. As shown in Table 4-1, different example antibodies may have a different HCDR1 sequence. The other CDR regions (HCDR2, HCDR3, LCDR1, LCDR2, and LCDR3) of the humanized antibody sequences (D6DD3D002-D6DD3D0016) may be the same as that of the mouse-derived antibody (numbered D6DD3D001).

TABLE 4-1
D6DD3D001

| | |
|---|---|
| VH | DVQLQQSGPGLVKPSQSLSLTCTVTGYSITSDSAWNWI |
| | RQFPGNRLEWMGYISYSGSTSYNPSLKSRISITRDTSK |
| | NQFFLQLNSVTTEDTATYYCVRGLRFAYWGQGTLVTVSS |
| | (SEQ ID NO: 1) |
| HCDR1 | GYSITSDSA (SEQ ID NO: 131) |
| Alterna- | GGSISSDSA (SEQ ID NO: 158) |
| tive | |
| HCDR1 | |
| HCDR2 | YISYSGSTSYNPSLKS (SEQ ID NO: 132) |
| HCDR3 | VRGLRFAY (SEQ ID NO: 133) |
| VL | DIVLTQSPASLAVSLGQRATISCRASESVDSYVNSFLH |
| | WYQQKPGQPPKLLIYRASNLQSGIPARFSGSGSRTDFT |
| | LTINPVEADDVATYYCQQSNEDPYTFGGGTKLEIK |
| | (SEQ ID NO: 3) |
| LCDR1 | ESVDSYVNSF (SEQ ID NO: 134) |
| LCDR2 | RASNLQS (SEQ ID NO: 135) |
| LCDR3 | QQSNEDPYT (SEQ ID NO: 136) |
| High-risk | NS |
| PTM site | |

### 4.1.2. Determination of Germline

Using the mouse antibody sequence, homologous proteins of the mouse antibody V region were searched in several human framework libraries (such as IMGT, Kabat, Igblast, etc.). The antibody sequences from several databases were compared to find sequences with high homology. Then, human germline framework sequences with high usage frequency were selected from the highly homologous sequences as candidate templates, which ensures better druggability of the final molecule.

### 4.1.3. CDR Grafting and Back Mutation

The humanization design adopted the CDR grafting method, which essentially involved replacing the mouse framework region (FR) with the human germline framework region (Framework region, FR) selected in the previous step, while retaining the murine CDR. The FR region cannot be replaced arbitrarily, as the sequence and conformation of the FR region often have some impact on the spatial configuration and function of the CDR region, and sometimes participate in antibody binding, such as regions in the FR region that are spatially close to the CDR region, typically within 5 amino acids or 3 amino acids of the FR region amino acids, which may have intramolecular interactions with the CDR region.

Simple CDR grafting often reduces the affinity of antigen-antibody binding. In order to reduce the adverse effects of humanization on the spatial configuration, activity, and function of the antibody, back mutation of some amino acids in the human framework region can be performed, i.e., to change them back to the amino acids at the corresponding site in the mouse FR region. The structure files of mouse antibodies are obtained from the antibody database, and the 3D structures of the light and heavy chains can be viewed. The distances, van der Waals forces, electrostatic interaction forces, and other interactions between the amino acids in the CDR region and other framework regions of the mouse antibody can be calculated, thereby locating the amino acids in the framework region that have strong interactions with the CDR region and listing these amino acids as candidates for back mutation. During this process, the balance between affinity/function/developability and immunogenicity/humanization degree can be considered. By comprehensively considering back mutations of different priority levels, candidate molecules with fewer back mutations and affinity/physiological function/developability in line with project expectations can be screened.

### 4.2. Design Results

On the antibody structure simulation and optimization analysis platform, the heavy chain sequence of Atibuclimab antibody was inputted, and homologous proteins in the V region were searched. The results from several databases were compared to determine five human germline framework sequences as templates for the heavy chain of Atibuclimab, and three human germline framework sequences as templates for the light chain of Atibuclimab. At the same time, amino acid mutations at PTM sites were predicted, and five PTM site-deleted sequences were designed.

After CDR grafting, several mutated sites were selected from each template-generated humanized sequence. Based on different mutated sites, multiple sequence plans were designed using the same template as a basis.

Selected results of the plans are shown in Table 4-2. In Table 4-2, “m VH” refers to mouse antibody heavy chain variable region; “hVH” refers to human antibody heavy chain variable region; “mVL” refers to mouse-derived antibody light chain variable region; “hVL” refers to humanized antibody light chain variable region; “mVHh” refers to humanized version of mVH.

In plans 1-3 for the mVHh humanized design, the mutated amino acid sites include R (AA #72) and V (AA #97); in plans 4 and 5 for the mVHh humanized design, the mutated amino acid sites include Y (AA #27) and T (AA #30), R (AA #72), V (AA #97). In plan 1 for the mVL-Humanized design, the mutated amino acid sites include R (AA #72) and V (AA #87); in plan 2 for the mVL-Humanized design, the mutated amino acid sites include L (AA #4) and R (AA #72); in plan 3 for the mVL-Humanized design, the mutated amino acid sites include L (AA #4), R (AA #72), and V (AA #87). Amino acids at the PTM sites (amino acid nos. 34, 35) included: NS (for Atibuclimab_hVL3 and Atibuclimab-mVL); DS (for Atibuclimab-mVL-V1); SS (for Atibuclimab-mVL-V2); TS (for Atibuclimab-mVL-V3); NA (for Atibuclimab-mVL-V4); and QS (for Atibuclimab-mVL-V5).

TABLE 4-2
Atibuclimab antibody humanized plan

| | | |
|---|---|---|
| | | Back |
| Name | Sequence | mutation |

Atibuclimab antibody-mVHh_Humanized_design

| Atibuclimab- | | (SEQ ID NO: 1) | n/a |
|---|---|---|---|
| mVH | | | |
| Template 1: | | QVQLQESGPGLVKPSQTLSLTCTVSGGSISSGGYYW | n/a |
| >IGHV4-31 | | SWIRQHPGKGLEWIGYIYYSGSTYYNPSLKSRVTISV | |
| | | DTSKNQFSLKLSSVTAADTAVYYCARYFDYWGQGT | |
| | | LVTVSS (SEQ ID NO: 137) | |
| Plan | Atibuc- | (SEQ ID NO: 5) | 2 |
| 1 | limab- | | |
| | hVH1 | | |
| Template 2: | | QVQLQESGPGLVKPSETLSLTCTVSGGSISSHYWSWI | n/a |
| >IGHV4-59 | | RQPPGKGLEWIGYIYYSGSTNYNPSLKSRVTISVDTS | |
| | | KNQFSLKLSSVTAADTAVYYCARYFDYWGQGTLV | |
| | | TVSS (SEQ ID NO: 138) | |
| Plan | Atibuc- | (SEQ ID NO: 9) | 2 |
| 2 | limab_ | | |
| | hVH2 | | |
| Template 3: | | QLQLQESGPGLVKPSETLSLTCTVSGGSISSSSYYWG | n/a |
| >IGHV4-39 | | WIRQPPGKGLEWIGSIYYSGSTYYNPSLKSRVTISVD | |
| | | TSKNQFSLKLSSVTAADTAVYYCARYFDYWGQGTL | |
| | | VTVSS (SEQ ID NO: 139) | |
| Plan | Atibuc- | (SEQ ID NO: 13) | 2 |
| 3 | limab_ | | |
| | hVH3 | | |
| Template 4: | | QVQLQESGPGLVKPSETLSLTCTVSGGSISSYYWSWI | n/a |
| >IGHV4-4 | | RQPPGKGLEWIGYIYTSGSTNYNPSLKSRVTISVDTS | |
| | | KNQFSLKLSSVTAADTAVYYCARYFDYWGQGTLV | |
| | | TVSS (SEQ ID NO: 140) | |
| Plan | Atibuc- | (SEQ ID NO: 17) | 4 |
| 4 | limab_ | | |
| | hVH4 | | |
| Template 5: | | QVQLQESGPGLVKPSQTLSLTCTVSGGSISSGDYYW | n/a |
| >IGHV4-30- | | SWIRQPPGKGLEWIGYIYYSGSTYYNPSLKSRVTISV | |
| 4*01, | | DTSKNQFSLKLSSVTAADTAVYYCARYFDYWGQGT | |
| IGHJ4*01 | | LVTVSS (SEQ ID NO: 141) | |
| Plan | Atibuc- | (SEQ ID NO: 21) | 4 |
| 5 | limab_ | | |
| | hVH5 | | |

Atibuclimab antibody-mVL_Humanized_design

| Atibuclimab- | | (SEQ ID NO: 3) | n/a |
|---|---|---|---|
| mVL | | | |
| Template 1: | | EIVLTQSPATLSLSPGERATLSCRASQSVSSYLAWYQ | n/a |
| >IGKV3-11 | | QKPGQAPRLLIYDASNRATGIPARFSGSGSGTDFTLT | |
| | | ISSLEPEDFAVYYCQQRSNWPYTFGQGTKLEIK (SEQ | |
| | | ID NO: 142) | |
| Plan | Atibuc- | (SEQ ID NO: 7) | 2 |
| 1 | limab_ | | |
| | hVL1 | | |
| Template 2: | | DIVMTQSPDSLAVSLGERATINCKSSQSVLYSSNNK | n/a |
| >IGKV4-1 | | NYLAWYQQKPGQPPKLLIYWASTRESGVPDRESGS | |
| | | GSGTDFTLTISSLQAEDVAVYYCQQYYSTPYTFGQG | |
| | | TKLEIK (SEQ ID NO: 143) | |
| Plan | Atibuc- | (SEQ ID NO: 27) | 2 |
| 2 | limab_ | | |
| | hVL2 | | |
| Template 3: | | DIQMTQSPSSLSASVGDRVTITCRASQSISSYLNWYQ | n/a |
| >IGKV3-39 | | QKPGKAPKLLIYAASSLQSGVPSRFSGSGSGTDFTLT | |
| | | ISSLQPEDFATYYCQQSYSTPYTFGQGTKLEIK (SEQ | |
| | | ID NO: 144) | |
| Plan | Atibuc- | (SEQ ID NO: 47) | 3 |
| 3 | limab_ | | |
| | hVL3 | | |

| | | |
|---|---|---|
| Atibuclimab- | (SEQ ID NO: 3) | n/a |
| mVL | | |

Atibuclimab antibody-mVL PTM site removal design

| | | |
|---|---|---|
| Atibuclimab- | DIVLTQSPASLAVSLGQRATISCRASESVDSYVDSFL | n/a |
| mVL-V1 | HWYQQKPGQPPKLLIYRASNLQSGIPARFSGSGSRT | |
| | DFTLTINPVEADDVATYYCQQSNEDPYTFGGGTKL | |
| | EIK (SEQ ID NO: 145) | |
| Atibuclimab- | DIVLTQSPASLAVSLGQRATISCRASESVDSYVSSFL | n/a |
| mVL-V2 | HWYQQKPGQPPKLLIYRASNLQSGIPARFSGSGSRT | |
| | DFTLTINPVEADDVATYYCQQSNEDPYTFGGGTKL | |
| | EIK (SEQ ID NO: 146) | |
| Atibuclimab- | DIVLTQSPASLAVSLGQRATISCRASESVDSYVTSFL | n/a |
| mVL-V3 | HWYQQKPGQPPKLLIYRASNLQSGIPARFSGSGSRT | |
| | DFTLTINPVEADDVATYYCQQSNEDPYTFGGGTKL | |
| | EIK (SEQ ID NO: 147) | |
| Atibuclimab- | DIVLTQSPASLAVSLGQRATISCRASESVDSYVNAF | n/a |
| mVL-V4 | LHWYQQKPGQPPKLLIYRASNLQSGIPARFSGSGSR | |
| | TDFTLTINPVEADDVATYYCQQSNEDPYTFGGGTK | |
| | LEIK (SEQ ID NO: 148) | |
| Atibuclimab- | DIVLTQSPASLAVSLGQRATISCRASESVDSYVQSFL | n/a |
| mVL-V5 | HWYQQKPGQPPKLLIYRASNLQSGIPARFSGSGSRT | |
| | DFTLTINPVEADDVATYYCQQSNEDPYTFGGGTKL | |
| | EIK (SEQ ID NO: 149) | |

### Example 5. Expression of Humanized Antibodies

### 5.1. Experimental Materials

Experimental reagents and consumables included cell culture medium, electroporation buffer, CHO cells, 1 mL electroporation cups, shaking flasks of various specifications, and pipettes of various specifications. Main instruments and equipment included a shaker (Taicang Huamei), centrifuge (Baiyang), ultrapure water system (Millipore), constant temperature incubator (BLUE PARD), electroporator, and water bath.

### 5.2. Experimental Methods

### 5.2.1. Gene Construction

The genes of the humanized heavy and light chain antibodies were separately cloned into plasmids. The amino acid sequences of the heavy and light chains are shown in Table 5-1. In Table 5-1, VH refers to the heavy chain variable region, VH+CH refers to the heavy chain, VL refers to the light chain variable region, and VL+CL refers to the light chain.

TABLE 5-1
Antibody No. D6DD3D001 (HU1127-mVH/mVL)

| | |
|---|---|
| VH | DVQLQQSGPGLVKPSQSLSLTCTVTGYSITSDSAWNWIRQFPGNRLEWM |
| | GYISYSGSTSYNPSLKSRISITRDTSKNQFFLQLNSVTTEDTATYYCVRGL |
| | RFAYWGQGTLVTVSS (SEQ ID NO: 1) |
| VH | DVQLQQSGPGLVKPSQSLSLTCTVTGYSITSDSAWNWIRQFPGNRLEWM |
| + | GYISYSGSTSYNPSLKSRISITRDTSKNQFFLQLNSVTTEDTATYYCVRGL |
| CH | RFAYWGQGTLVTVSSPSVFLFPPKPKDTLMISRTPEVTCVVVDVSHEDPE |
| | VKFNWYVDGVEVHNAKTKPREEQYNSTYRVVSVLTVLHQDWLNGKE |
| | YKCKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKSRW |
| | QQGNVFSCSVMHEALHNHYTQKSLSLSPGK (SEQ ID NO: 2) |
| VL | DIVLTQSPASLAVSLGQRATISCRASESVDSYVNSFLHWYQQKPGQPPKL |
| | LIYRASNLQSGIPARFSGSGSRTDFTLTINPVEADDVATYYCQQSNEDPY |
| | TFGGGTKLEIK (SEQ ID NO: 3) |
| VL | DIVLTQSPASLAVSLGQRATISCRASESVDSYVNSFLHWYQQKPGQPPKL |
| + | LIYRASNLQSGIPARFSGSGSRTDFTLTINPVEADDVATYYCQQSNEDPY |
| CL | TFGGGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 4) |

Antibody No. D6DD3D002 (HU1127-hVH1/hVL1)

| | |
|---|---|
| VH | QVQLQESGPGLVKPSQTLSLTCTVSGGSISSDSAWNWIRQHPGKGLEWI |
| | GYISYSGSTSYNPSLKSRVTISRDTSKNQFSLKLSSVTAADTAVYYCVRG |
| | LRFAYWGQGTLVTVSS (SEQ ID NO: 5) |
| VH | QVQLQESGPGLVKPSQTLSLTCTVSGGSISSDSAWNWIRQHPGKGLEWI |
| + | GYISYSGSTSYNPSLKSRVTISRDTSKNQFSLKLSSVTAADTAVYYCVRG |
| CH | LRFAYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 6) |
| VL | EIVLTQSPATLSLSPGERATLSCRASESVDSYVNSFLHWYQQKPGQAPRL |
| | LIYRASNLQSGIPARFSGSGSRTDFTLTISSLEPEDVAVYYCQQSNEDPYT |
| | FGQGTKLEIK (SEQ ID NO: 7) |
| VL | EIVLTQSPATLSLSPGERATLSCRASESVDSYVNSFLHWYQQKPGQAPRL |
| + | LIYRASNLQSGIPARFSGSGSRTDFTLTISSLEPEDVAVYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 8) |

Antibody No. D6DD3D003 (HU1127-hVH2/hVL1)

| | |
|---|---|
| VH | QVQLQESGPGLVKPSETLSLTCTVSGGSISSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 9) |
| VH | QVQLQESGPGLVKPSETLSLTCTVSGGSISSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 10) |
| VL | EIVLTQSPATLSLSPGERATLSCRASESVDSYVNSFLHWYQQKPGQAPRL |
| | LIYRASNLQSGIPARFSGSGSRTDFTLTISSLEPEDVAVYYCQQSNEDPYT |
| | FGQGTKLEIK (SEQ ID NO: 11) |
| VL | EIVLTQSPATLSLSPGERATLSCRASESVDSYVNSFLHWYQQKPGQAPRL |
| + | LIYRASNLQSGIPARFSGSGSRTDFTLTISSLEPEDVAVYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 12) |

Antibody No. D6DD3D004 (HU1127-hVH3/hVL1)

| | |
|---|---|
| VH | QLQLQESGPGLVKPSETLSLTCTVSGGSISSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 13) |
| VH | QLQLQESGPGLVKPSETLSLTCTVSGGSISSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 14) |
| VL | EIVLTQSPATLSLSPGERATLSCRASESVDSYVNSFLHWYQQKPGQAPRL |
| | LIYRASNLQSGIPARFSGSGSRTDFTLTISSLEPEDVAVYYCQQSNEDPYT |
| | FGQGTKLEIK (SEQ ID NO: 15) |
| VL | EIVLTQSPATLSLSPGERATLSCRASESVDSYVNSFLHWYQQKPGQAPRL |
| + | LIYRASNLQSGIPARFSGSGSRTDFTLTISSLEPEDVAVYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 16) |

Antibody No. D6DD3D005 (HU1127-hVH4/hVL1)

| | |
|---|---|
| VH | QVQLQESGPGLVKPSETLSLTCTVSGYSITSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 17) |
| VH | QVQLQESGPGLVKPSETLSLTCTVSGYSITSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 18) |
| VL | EIVLTQSPATLSLSPGERATLSCRASESVDSYVNSFLHWYQQKPGQAPRL |
| | LIYRASNLQSGIPARFSGSGSRTDFTLTISSLEPEDVAVYYCQQSNEDPYT |
| | FGQGTKLEIK (SEQ ID NO: 19) |
| VL | EIVLTQSPATLSLSPGERATLSCRASESVDSYVNSFLHWYQQKPGQAPRL |
| + | LIYRASNLQSGIPARFSGSGSRTDFTLTISSLEPEDVAVYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 20) |

Antibody No. D6DD3D006 (HU1127-hVH5/hVL1)

| | |
|---|---|
| VH | QVQLQESGPGLVKPSQTLSLTCTVSGYSITSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 21) |
| VH | QVQLQESGPGLVKPSQTLSLTCTVSGYSITSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 22) |
| VL | EIVLTQSPATLSLSPGERATLSCRASESVDSYVNSFLHWYQQKPGQAPRL |
| | LIYRASNLQSGIPARFSGSGSRTDFTLTISSLEPEDVAVYYCQQSNEDPYT |
| | FGQGTKLEIK (SEQ ID NO: 23) |
| VL | EIVLTQSPATLSLSPGERATLSCRASESVDSYVNSFLHWYQQKPGQAPRL |
| + | LIYRASNLQSGIPARFSGSGSRTDFTLTISSLEPEDVAVYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 24) |

Antibody No. D6DD3D007 (HU1127-hVH1/hVL2)

| | |
|---|---|
| VH | QVQLQESGPGLVKPSQTLSLTCTVSGGSISSDSAWNWIRQHPGKGLEWI |
| | GYISYSGSTSYNPSLKSRVTISRDTSKNQFSLKLSSVTAADTAVYYCVRG |
| | LRFAYWGQGTLVTVSS (SEQ ID NO: 25) |
| VH | QVQLQESGPGLVKPSQTLSLTCTVSGGSISSDSAWNWIRQHPGKGLEWI |
| + | GYISYSGSTSYNPSLKSRVTISRDTSKNQFSLKLSSVTAADTAVYYCVRG |
| CH | LRFAYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 26) |
| VL | DIVLTQSPDSLAVSLGERATINCRASESVDSYVNSFLHWYQQKPGQPPKL |
| | LIYRASNLQSGVPDRFSGSGSRTDFTLTISSLQAEDVAVYYCQQSNEDPY |
| | TFGQGTKLEIK (SEQ ID NO: 27) |
| VL | DIVLTQSPDSLAVSLGERATINCRASESVDSYVNSFLHWYQQKPGQPPKL |
| + | LIYRASNLQSGVPDRESGSGSRTDFTLTISSLQAEDVAVYYCQQSNEDPY |
| CL | TFGQGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 28) |

Antibody No. D6DD3D008 (HU1127-hVH2/hVL2)

| | |
|---|---|
| VH | QVQLQESGPGLVKPSETLSLTCTVSGGSISSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 29) |
| VH | QVQLQESGPGLVKPSETLSLTCTVSGGSISSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 30) |
| VL | DIVLTQSPDSLAVSLGERATINCRASESVDSYVNSFLHWYQQKPGQPPKL |
| | LIYRASNLQSGVPDRFSGSGSRTDFTLTISSLQAEDVAVYYCQQSNEDPY |
| | TFGQGTKLEIK (SEQ ID NO: 31) |
| VL | DIVLTQSPDSLAVSLGERATINCRASESVDSYVNSFLHWYQQKPGQPPKL |
| + | LIYRASNLQSGVPDRFSGSGSRTDFTLTISSLQAEDVAVYYCQQSNEDPY |
| CL | TFGQGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 32) |

Antibody No. D6DD3D009 (HU1127-hVH3/hVL2)

| | |
|---|---|
| VH | QLQLQESGPGLVKPSETLSLTCTVSGGSISSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 33) |
| VH | QLQLQESGPGLVKPSETLSLTCTVSGGSISSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 34) |
| VL | DIVLTQSPDSLAVSLGERATINCRASESVDSYVNSFLHWYQQKPGQPPKL |
| | LIYRASNLQSGVPDRESGSGSRTDFTLTISSLQAEDVAVYYCQQSNEDPY |
| | TFGQGTKLEIK (SEQ ID NO: 35) |
| VL | DIVLTQSPDSLAVSLGERATINCRASESVDSYVNSFLHWYQQKPGQPPKL |
| + | LIYRASNLQSGVPDRFSGSGSRTDFTLTISSLQAEDVAVYYCQQSNEDPY |
| CL | TFGQGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 36) |

Antibody No. D6DD3D010 (HU1127-hVH4/hVL2)

| | |
|---|---|
| VH | QVQLQESGPGLVKPSETLSLTCTVSGYSITSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 37) |
| VH | QVQLQESGPGLVKPSETLSLTCTVSGYSITSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 38) |
| VL | DIVLTQSPDSLAVSLGERATINCRASESVDSYVNSFLHWYQQKPGQPPKL |
| | LIYRASNLQSGVPDRESGSGSRTDFTLTISSLQAEDVAVYYCQQSNEDPY |
| | TFGQGTKLEIK (SEQ ID NO: 39) |
| VL | DIVLTQSPDSLAVSLGERATINCRASESVDSYVNSFLHWYQQKPGQPPKL |
| + | LIYRASNLQSGVPDRFSGSGSRTDFTLTISSLQAEDVAVYYCQQSNEDPY |
| CL | TFGQGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 40) |

Antibody No. D6DD3D011 (HU1127-hVH5/hVL2)

| | |
|---|---|
| VH | QVQLQESGPGLVKPSQTLSLTCTVSGYSITSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 41) |
| VH | QVQLQESGPGLVKPSQTLSLTCTVSGYSITSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 42) |
| VL | DIVLTQSPDSLAVSLGERATINCRASESVDSYVNSFLHWYQQKPGQPPKL |
| | LIYRASNLQSGVPDRFSGSGSRTDFTLTISSLQAEDVAVYYCQQSNEDPY |
| | TFGQGTKLEIK (SEQ ID NO: 43) |
| VL | DIVLTQSPDSLAVSLGERATINCRASESVDSYVNSFLHWYQQKPGQPPKL |
| + | LIYRASNLQSGVPDRFSGSGSRTDFTLTISSLQAEDVAVYYCQQSNEDPY |
| CL | TFGQGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVYAC |
| | EVTHQGLSSPVTKSFNRGEC (SEQ ID NO: 44) |

Antibody No. D6DD3D012 (HU1127-hVH1/hVL3)

| | |
|---|---|
| VH | QVQLQESGPGLVKPSQTLSLTCTVSGGSISSDSAWNWIRQHPGKGLEWI |
| | GYISYSGSTSYNPSLKSRVTISRDTSKNQFSLKLSSVTAADTAVYYCVRG |
| | LRFAYWGQGTLVTVSS (SEQ ID NO: 45) |
| VH | QVQLQESGPGLVKPSQTLSLTCTVSGGSISSDSAWNWIRQHPGKGLEWI |
| + | GYISYSGSTSYNPSLKSRVTISRDTSKNQFSLKLSSVTAADTAVYYCVRG |
| CH | LRFAYWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 46) |
| VL | DIQLTQSPSSLSASVGDRVTITCRASESVDSYVNSFLHWYQQKPGKAPKL |
| | LIYRASNLQSGVPSRFSGSGSRTDFTLTISSLQPEDVATYYCQQSNEDPYT |
| | FGQGTKLEIK (SEQ ID NO: 47) |
| VL | DIQLTQSPSSLSASVGDRVTITCRASESVDSYVNSFLHWYQQKPGKAPKL |
| + | LIYRASNLQSGVPSRFSGSGSRTDFTLTISSLQPEDVATYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 48) |

Antibody No. D6DD3D013 (HU1127-hVH2/hVL3

| | |
|---|---|
| VH | QVQLQESGPGLVKPSETLSLTCTVSGGSISSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 49) |
| VH | QVQLQESGPGLVKPSETLSLTCTVSGGSISSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 50) |
| VL | DIQLTQSPSSLSASVGDRVTITCRASESVDSYVNSFLHWYQQKPGKAPKL |
| | LIYRASNLQSGVPSRFSGSGSRTDFTLTISSLQPEDVATYYCQQSNEDPYT |
| | FGQGTKLEIK (SEQ ID NO: 51) |
| VL | DIQLTQSPSSLSASVGDRVTITCRASESVDSYVNSFLHWYQQKPGKAPKL |
| + | LIYRASNLQSGVPSRFSGSGSRTDFTLTISSLQPEDVATYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 52) |

Antibody No. D6DD3D014 (HU1127-hVH3/hVL3)

| | |
|---|---|
| VH | QLQLQESGPGLVKPSETLSLTCTVSGGSISSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 53) |
| VH | QLQLQESGPGLVKPSETLSLTCTVSGGSISSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 54) |
| VL | DIQLTQSPSSLSASVGDRVTITCRASESVDSYVNSFLHWYQQKPGKAPKL |
| | LIYRASNLQSGVPSRFSGSGSRTDFTLTISSLQPEDVATYYCQQSNEDPYT |
| | FGQGTKLEIK (SEQ ID NO: 55) |
| VL | DIQLTQSPSSLSASVGDRVTITCRASESVDSYVNSFLHWYQQKPGKAPKL |
| + | LIYRASNLQSGVPSRFSGSGSRTDFTLTISSLQPEDVATYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 56) |

Antibody No. D6DD3D015 (HU1127-hVH4/hVL3)

| | |
|---|---|
| VH | QVQLQESGPGLVKPSETLSLTCTVSGYSITSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 57) |
| VH | QVQLQESGPGLVKPSETLSLTCTVSGYSITSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 58) |
| VL | DIQLTQSPSSLSASVGDRVTITCRASESVDSYVNSFLHWYQQKPGKAPKL |
| | LIYRASNLQSGVPSRFSGSGSRTDFTLTISSLQPEDVATYYCQQSNEDPYT |
| | FGQGTKLEIK (SEQ ID NO: 59) |
| VL | DIQLTQSPSSLSASVGDRVTITCRASESVDSYVNSFLHWYQQKPGKAPKL |
| + | LIYRASNLQSGVPSRFSGSGSRTDFTLTISSLQPEDVATYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 60) |

Antibody No. D6DD3D016 (HU1127-hVH5/hVL3)

| | |
|---|---|
| VH | QVQLQESGPGLVKPSQTLSLTCTVSGYSITSDSAWNWIRQPPGKGLEWIG |
| | YISYSGSTSYNPSLKSRVTISRDTSKNQFSLKLSSVTAADTAVYYCVRGL |
| | RFAYWGQGTLVTVSS (SEQ ID NO: 61) |
| VH | QVQLQESGPGLVKPSQTLSLTCTVSGYSITSDSAWNWIRQPPGKGLEWIG |
| + | YISYSGSTSYNPSLKSRVTISRDTSKNQFSLKLSSVTAADTAVYYCVRGL |
| CH | RFAYWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK (SEQ ID NO: 62) |
| VL | DIQLTQSPSSLSASVGDRVTITCRASESVDSYVNSFLHWYQQKPGKAPKL |
| | LIYRASNLQSGVPSRFSGSGSRTDFTLTISSLQPEDVATYYCQQSNEDPYT |
| | FGQGTKLEIK (SEQ ID NO: 63) |
| VL | DIQLTQSPSSLSASVGDRVTITCRASESVDSYVNSFLHWYQQKPGKAPKL |
| + | LIYRASNLQSGVPSRFSGSGSRTDFTLTISSLQPEDVATYYCQQSNEDPYT |
| CL | FGQGTKLEIKRTVAAPSVFIFPPSDEQLKSGTASVVCLLNNFYPREAKVQ |
| | WKVDNALQSGNSQESVTEQDSKDSTYSLSSTLTLSKADYEKHKVYACE |
| | VTHQGLSSPVTKSFNRGEC (SEQ ID NO: 64) |

Heavy chain and light chain amino acid sequences can include a signal peptide having an amino acid sequence as set forth in SEQ ID NO: 129 (MHSSALLCCLVLLTGVRA).

### 5.2.2. Antibody Expression and Purification

Fifteen humanized antibodies were obtained by pairing the light and heavy chains, and together with the parental antibody, a total of 16 pairs of paired antibodies were obtained. About 145 million cells were centrifuged to remove the supernatant, and about 0.5 mL of electroporation solution was added to the cells. After mixing, an appropriate amount of paired plasmids (concentration 500 ng/μL) were added. After thorough mixing, 1 mL of the above cell and plasmid suspension was added to a 1 ml electroporation cup, which was then placed in an electroporator for electroporation. After electroporation, the cells in the electroporation cup were transferred into a shaking flask containing 20 mL of pre-prepared culture medium, and incubated for 40 minutes. After incubation, the shaking flask was placed in a 37° C., 270 rpm, 8% CO2 incubator and cultured for 4 days. The supernatant was collected by centrifugation (8000 rpm, 5 min), and the obtained supernatant was purified by Protein A affinity chromatography.

### 5.2.3. Experimental Results

The purity of 15 humanized Atibuclimab antibodies (numbered D6DD3D002-D6DD3D0016) and mouse-derived antibody (numbered D6DD3D001) were detected by size exclusion chromatography (SEC) and sodium dodecyl sulfate-polyacrylamide gel electrophoresis (SDS-PAGE). By SDS-Page, the antibodies had a purity of >95%. Table 5-2 shows selected results such as concentration (Conc.), purity, and back mutation number (BMN). In Table 5-2, purity by SEC was detected at an absorbance of 214 nm and at an absorbance of 280 nm. Purity values in Table 5-2 are in units of percent, %.

TABLE 5-2

| | | Purity | Purity | BMN |
|---|---|---|---|---|
| | Conc., | (SEC, | (SEC, | (H + |
| Antibody No. (Name) | mg/mL | 214 nm) | 280 nm) | L) |
| D6DD3D001 (HU1127-mVH/mVL) | 1.2 | 98.99 | 98.686 | — |
| D6DD3D002 (HU1127-hVH1/hVL1) | 0.33 | 98.956 | 99.199 | 2 + 2 |
| D6DD3D003 (HU1127-hVH2/hVL1) | 0.47 | 98.332 | 98.319 | 2 + 2 |
| D6DD3D004 (HU1127-hVH3/hVL1) | 0.61 | 98.151 | 98.091 | 2 + 2 |
| D6DD3D005 (HU1127-hVH4/hVL1) | 0.44 | 100 | 100 | 4 + 2 |
| D6DD3D006 (HU1127-hVH5/hVL1) | 0.5 | 100 | 100 | 4 + 2 |
| D6DD3D007 (HU1127-hVH1/hVL2) | 0.49 | 99.167 | 99.306 | 2 + 2 |
| D6DD3D008 (HU1127-hVH2/hVL2) | 0.6 | 98.772 | 98.805 | 2 + 2 |
| D6DD3D009 (HU1127-hVH3/hVL2) | 0.54 | 98.955 | 99.011 | 2 + 2 |
| D6DD3D010 (HU1127-hVH4/hVL2) | 0.44 | 99.113 | 99.065 | 4 + 2 |
| D6DD3D011 (HU1127-hVH5/hVL2) | 0.53 | 99.726 | 99.864 | 4 + 2 |
| D6DD3D012 (HU1127-hVH1/hVL3) | 1.2 | 100 | 99.656 | 2 + 3 |
| D6DD3D013 (HU1127-hVH2/hVL3) | 1.53 | 98.965 | 99.098 | 2 + 3 |
| D6DD3D014 (HU1127-hVH3/hVL3) | 1.25 | 99.079 | 98.977 | 2 + 3 |
| D6DD3D015 (HU1127-hVH4/hVL3) | 1.26 | 99.723 | 99.421 | 4 + 3 |
| D6DD3D016 (HU1127-hVH5/hVL3) | 1.46 | 100 | 100 | 4 + 3 |

The data in Table 5-2 indicates that the samples are ready for further investigations such as binding affinity and other experiments.

### 5.3. Example Nucleotide Sequences

Generation of the genetic construct for the antibodies was performed as follows, with IgG1 as an example.

### 5.3.1. Example: IgG1 Recombinant Antibody Preparation

### CHO Electroporation Expression

Experimental materials included a shaker, centrifuge, water bath, electroporator, 1 mL electroporation cuvette, various sizes of shaking flasks, various sizes of pipettes, electroporation buffer.

### Experimental Method

Cell culture operations are shown in Table 5-3.

TABLE 5-3

| | | Density (before | Density (after |
|---|---|---|---|
| | | passaging), | passaging), |
| Operation | Conditions | cells/mL | cells/mL |
| Cell Revival | 120 rpm; 8% CO₂; | 0 | 0.3 × 10⁶ |
| | 37° C. culture | | |
| First cell | 120 rpm; 8% CO₂; | 4.5 × 10⁶ | 0.3 × 10⁶ |
| passage | 37° C. culture | | |
| Second cell | 120 rpm; 8% CO₂; | 5.85 × 10⁶ | 0.3 × 10⁶ |
| passage | 37° C. culture | | |
| Third cell | 120 rpm; 8% CO₂; | 6.52 × 10⁶ | 0.3 × 10⁶ |
| passage | 37° C. culture | | |
| Transfection | 270/120 rpm; 8% CO₂; | 6.85 × 10⁶ | 0.3 × 10⁶ |
| | 37° C. culture | | |

Transfection and expression was conducted with 20 mL expression according to the following procedure: (a) Take 145 million cells, centrifuge and discard the supernatant. (b) Add about 0.5 mL of transfection buffer to the cells, mix well and then add an appropriate amount of plasmid (concentration 500 ng/μL). (c) After the cell-plasmid suspension is well mixed, take 1 mL and add it to a 1 mL electroporation tube, and place the electroporation tube into the electroporator and perform electroporation. (d) After the electroporation is completed, distribute the cells inside the electroporation tube into a prepared flask containing 20 mL culture medium and incubate for 40 minutes without shaking. (e) After incubation, put the flask in a 37° C., 270 rpm, 8% CO₂ culture, add additional medium/butyric acid/split antibiotics after 24 hours, and continue to culture for 3-6 days.

### Antibody Purification:

Experimental materials included a stirrer, 1×PBS, sodium citrate, Protein A pre-packed column, various sizes of centrifuge tubes.

Experimental Method: Protein A Affinity Chromatography Column Purification.

Equilibrating the column: 1×PBS, flow rate of 1 mL/min, 20 mL.

Sample application: flow rate of 1 mL/min.

Washing impurities: 1×PBS, flow rate of 1 mL/min, 20 mL.

Elution: Citrate buffer (pH 3.2), 1 mL/min, collection by tubes, approximately 500 μL per tube. Collect 10 tubes in total and read the absorbance at 280 nm with a NanoDrop instrument.

Dialysis: Absorb the high concentration protein into a dialysis bag and dialyze it in a beaker with 1×PBS.

As stated above, the genes of the humanized heavy and light chain antibodies were separately cloned into plasmids. To generate the genetic construct for the remaining antibodies, a similar procedure as described above was performed.

The nucleotide sequences that encode selected antibodies (or fragments thereof) described herein are shown in Table 5-4. VH refers to the nucleotide sequence encoding the heavy chain variable region, VH+CH refers to the nucleotide sequence encoding the heavy chain, VL refers to the nucleotide sequence encoding the light chain variable region, and VL+CL refers to the nucleotide sequence encoding the light chain.

TABLE 5-4
Example nucleotide sequences encoding example antibodies
Antibody No. D6DD3D001 (HU1127-mVH/mVL)

| | |
|---|---|
| VH | GATGTGCAGCTGCAGCAGAGCGGACCTGGATTGGTGAAGCCTTCCCA |
| | GTCCCTGAGCCTGACCTGTACCGTGACCGGATATTCTATTACTTCCGA |
| | CTCCGCTTGGAACTGGATTAGGCAGTTCCCCGGCAACAGGCTGGAAT |
| | GGATGGGATATATCAGCTACTCCGGCTCCACCTCCTACAACCCCTCCT |
| | TGAAGTCCCGGATCTCCATCACCCGCGATACCTCCAAGAACCAGTTC |
| | TTCCTGCAGCTCAACTCTGTGACCACCGAGGACACCGCCACCTACTA |
| | CTGCGTGAGGGGACTGCGATTCGCCTACTGGGGACAGGGTACCCTCG |
| | TTACCGTGTCTAGC (SEQ ID NO: 65) |
| VH | GATGTGCAGCTGCAGCAGAGCGGACCTGGATTGGTGAAGCCTTCCCA |
| + | GTCCCTGAGCCTGACCTGTACCGTGACCGGATATTCTATTACTTCCGA |
| CH | CTCCGCTTGGAACTGGATTAGGCAGTTCCCCGGCAACAGGCTGGAAT |
| | GGATGGGATATATCAGCTACTCCGGCTCCACCTCCTACAACCCCTCCT |
| | TGAAGTCCCGGATCTCCATCACCCGCGATACCTCCAAGAACCAGTTC |
| | TTCCTGCAGCTCAACTCTGTGACCACCGAGGACACCGCCACCTACTA |
| | CTGCGTGAGGGGACTGCGATTCGCCTACTGGGGACAGGGTACCCTCG |
| | TTACCGTGTCTAGCGCTAGCACCAAGGGCCCATCGGTCTTCCCCCTGG |
| | CACCCTCCTCCAAGAGCACCTCTGGGGGCACAGCGGCCCTGGGCTGC |
| | CTGGTCAAGGACTACTTCCCCGAACCGGTGACGGTGTCGTGGAACTC |
| | AGGCGCCCTGACCAGCGGCGTGCACACCTTCCCGGCTGTCCTACAGT |
| | CCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 66) |
| VL | GATATCGTGCTGACCCAGTCCCCCGCTTCCTTGGCTGTTTCCCTGGGA |
| | CAGAGGGCCACCATCTCCTGTAGGGCTTCCGAGTCCGTGGACTCCTA |
| | CGTGAACAGCTTTTTGCACTGGTACCAGCAGAAGCCCGGCCAGCCTC |
| | CTAAGTTGCTCATCTACAGGGCTTCCAACCTGCAGTCCGGCATTCCAG |
| | CCCGTTTTTCCGGGTCCGGTTCCAGAACCGACTTCACCCTCACCATCA |
| | ACCCCGTCGAGGCAGACGACGTCGCTACTTACTACTGCCAGCAGTCC |
| | AACGAAGATCCTTATACCTTCGGAGGCGGCACCAAGCTCGAAATCAA |
| | A (SEQ ID NO: 67) |
| VL | GATATCGTGCTGACCCAGTCCCCCGCTTCCTTGGCTGTTTCCCTGGGA |
| + | CAGAGGGCCACCATCTCCTGTAGGGCTTCCGAGTCCGTGGACTCCTA |
| CL | CGTGAACAGCTTTTTGCACTGGTACCAGCAGAAGCCCGGCCAGCCTC |
| | CTAAGTTGCTCATCTACAGGGCTTCCAACCTGCAGTCCGGCATTCCAG |
| | CCCGTTTTTCCGGGTCCGGTTCCAGAACCGACTTCACCCTCACCATCA |
| | ACCCCGTCGAGGCAGACGACGTCGCTACTTACTACTGCCAGCAGTCC |
| | AACGAAGATCCTTATACCTTCGGAGGCGGCACCAAGCTCGAAATCAA |
| | ACGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 68) |

Antibody No. D6DD3D002 (HU1127-hVH1/hVL1)

| | |
|---|---|
| VH | CAGGTTCAGCTCCAGGAGTCAGGCCCAGGACTCGTTAAACCCTCCCA |
| | GACCCTCTCCCTCACCTGCACAGTCTCCGGTGGAAGCATCTCCTCCGA |
| | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCC (SEQ ID NO: 69) |
| VH | CAGGTTCAGCTCCAGGAGTCAGGCCCAGGACTCGTTAAACCCTCCCA |
| + | GACCCTCTCCCTCACCTGCACAGTCTCCGGTGGAAGCATCTCCTCCGA |
| CH | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCCGCTAGCACCAAGGGCCCATCGGTCTTCCCCCTGG |
| | CACCCTCCTCCAAGAGCACCTCTGGGGGCACAGCGGCCCTGGGCTGC |
| | CTGGTCAAGGACTACTTCCCCGAACCGGTGACGGTGTCGTGGAACTC |
| | AGGCGCCCTGACCAGCGGCGTGCACACCTTCCCGGCTGTCCTACAGT |
| | CCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 70) |
| VL | GAAATCGTGCTGACCCAGTCCCCCGCTACCTTGAGTCTGTCCCCTGGA |
| | GAGAGAGCCACCCTGTCTTGTAGGGCTAGTGAGAGCGTGGACTCCTA |
| | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | G (SEQ ID NO: 71) |
| VL | GAAATCGTGCTGACCCAGTCCCCCGCTACCTTGAGTCTGTCCCCTGGA |
| + | GAGAGAGCCACCCTGTCTTGTAGGGCTAGTGAGAGCGTGGACTCCTA |
| CL | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 72) |

Antibody No. D6DD3D003 (HU1127-hVH2/hVL1)

| | |
|---|---|
| VH | CAGGTTCAGCTCCAGGAGTCAGGCCCAGGACTCGTTAAACCCTCCGA |
| | GACACTCTCCCTGACATGCACAGTCTCCGGCGGAAGCATCTCCTCCG |
| | ACTCAGCTTGGAACTGGATCAGGCAGCCCCCCGGAAAGGGTTTGGAG |
| | TGGATTGGCTATATCTCCTACTCCGGCTCCACCTCCTACAACCCAAGC |
| | CTGAAAAGCCGAGTGACCATCTCTAGGGACACCTCCAAGAACCAGTT |
| | CTCCCTCAAACTGAGCAGCGTGACCGCCGCTGACACCGCTGTTTACT |
| | ACTGCGTCCGCGGCCTGAGGTTCGCTTACTGGGGACAGGGAACCCTC |
| | GTCACCGTGTCTAGC (SEQ ID NO: 73) |
| VH | CAGGTTCAGCTCCAGGAGTCAGGCCCAGGACTCGTTAAACCCTCCGA |
| + | GACACTCTCCCTGACATGCACAGTCTCCGGCGGAAGCATCTCCTCCG |
| CH | ACTCAGCTTGGAACTGGATCAGGCAGCCCCCCGGAAAGGGTTTGGAG |
| | TGGATTGGCTATATCTCCTACTCCGGCTCCACCTCCTACAACCCAAGC |
| | CTGAAAAGCCGAGTGACCATCTCTAGGGACACCTCCAAGAACCAGTT |
| | CTCCCTCAAACTGAGCAGCGTGACCGCCGCTGACACCGCTGTTTACT |
| | ACTGCGTCCGCGGCCTGAGGTTCGCTTACTGGGGACAGGGAACCCTC |
| | GTCACCGTGTCTAGCGCTAGCACCAAGGGCCCATCGGTCTTCCCCCT |
| | GGCACCCTCCTCCAAGAGCACCTCTGGGGGCACAGCGGCCCTGGGCT |
| | GCCTGGTCAAGGACTACTTCCCCGAACCGGTGACGGTGTCGTGGAAC |
| | TCAGGCGCCCTGACCAGCGGCGTGCACACCTTCCCGGCTGTCCTACA |
| | GTCCTCAGGACTCTACTCCCTCAGCAGCGTGGTGACCGTGCCCTCCA |
| | GCAGCTTGGGCACCCAGACCTACATCTGCAACGTGAATCACAAGCCC |
| | AGCAACACCAAGGTGGACAAGAAAGTTGAGCCCAAATCTTGTGACA |
| | AAACTCACACATGCCCACCGTGCCCAGCACCTGAACTCCTGGGGGGA |
| | CCGTCAGTCTTCCTCTTCCCCCCAAAACCCAAGGACACCCTCATGATC |
| | TCCCGGACCCCCGAGGTCACATGCGTGGTGGTGGACGTGAGCCACGA |
| | AGACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCC |
| | ATCGAGAAAACCATCTCCAAAGCCAAAGGGCAGCCCCGAGAACCAC |
| | AGGTGTACACCCTGCCCCCATCCCGGGAGGAGATGACCAAGAACCA |
| | GGTCAGCCTGACCTGCCTGGTCAAAGGCTTCTATCCCAGCGACATCG |
| | CCGTGGAGTGGGAGAGCAATGGGCAGCCGGAGAACAACTACAAGAC |
| | CACGCCTCCCGTGCTGGACTCCGACGGCTCCTTCTTCCTCTACAGCAA |
| | GCTCACCGTGGACAAGAGCAGGTGGCAGCAGGGGAACGTCTTCTCAT |
| | GCTCCGTGATGCATGAGGCTCTGCACAACCACTACACGCAGAAGAGC |
| | CTCTCCCTGTCTCCGGGTAAA (SEQ ID NO: 74) |
| VL | GAAATCGTGCTGACCCAGTCCCCCGCTACCTTGAGTCTGTCCCCTGGA |
| | GAGAGAGCCACCCTGTCTTGTAGGGCTAGTGAGAGCGTGGACTCCTA |
| | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | G (SEQ ID NO: 75) |
| VL | GAAATCGTGCTGACCCAGTCCCCCGCTACCTTGAGTCTGTCCCCTGGA |
| + | GAGAGAGCCACCCTGTCTTGTAGGGCTAGTGAGAGCGTGGACTCCTA |
| CL | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 76) |

Antibody No. D6DD3D004 (HU1127-hVH3/hVL1)

| | |
|---|---|
| VH | CAGTTGCAGCTCCAGGAGTCCGGTCCAGGACTCGTTAAACCCTCCGA |
| | GACACTCTCCCTCACCTGCACAGTCTCCGGCGGAAGTATCTCCTCCGA |
| | CTCCGCTTGGAACTGGATCAGGCAGCCTCCCGGTAAGGGGTTGGAGT |
| | GGATCGGATATATCTCCTACAGCGGCTCCACCTCCTACAATCCCAGTC |
| | TGAAGTCCAGAGTGACCATCTCCCGCGACACCTCCAAGAACCAGTTC |
| | TCCCTCAAACTGTCCAGCGTGACCGCCGCAGACACCGCTGTTTACTA |
| | CTGCGTGCGCGGCCTGAGATTTGCCTACTGGGGACAGGGCACCCTCG |
| | TGACAGTGTCTAGC (SEQ ID NO: 77) |
| VH | CAGTTGCAGCTCCAGGAGTCCGGTCCAGGACTCGTTAAACCCTCCGA |
| + | GACACTCTCCCTCACCTGCACAGTCTCCGGCGGAAGTATCTCCTCCGA |
| CH | CTCCGCTTGGAACTGGATCAGGCAGCCTCCCGGTAAGGGGTTGGAGT |
| | GGATCGGATATATCTCCTACAGCGGCTCCACCTCCTACAATCCCAGTC |
| | TGAAGTCCAGAGTGACCATCTCCCGCGACACCTCCAAGAACCAGTTC |
| | TCCCTCAAACTGTCCAGCGTGACCGCCGCAGACACCGCTGTTTACTA |
| | CTGCGTGCGCGGCCTGAGATTTGCCTACTGGGGACAGGGCACCCTCG |
| | TGACAGTGTCTAGCGCTAGCACCAAGGGCCCATCGGTCTTCCCCCTG |
| | GCACCCTCCTCCAAGAGCACCTCTGGGGGCACAGCGGCCCTGGGCTG |
| | CCTGGTCAAGGACTACTTCCCCGAACCGGTGACGGTGTCGTGGAACT |
| | CAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 78) |
| VL | GAAATCGTGCTGACCCAGTCCCCCGCTACCTTGAGTCTGTCCCCTGGA |
| | GAGAGAGCCACCCTGTCTTGTAGGGCTAGTGAGAGCGTGGACTCCTA |
| | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | G (SEQ ID NO: 79) |
| VL | GAAATCGTGCTGACCCAGTCCCCCGCTACCTTGAGTCTGTCCCCTGGA |
| + | GAGAGAGCCACCCTGTCTTGTAGGGCTAGTGAGAGCGTGGACTCCTA |
| CL | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 80) |

Antibody No. D6DD3D005 (HU1127-hVH4/hVL1)

| | |
|---|---|
| VH | CAGGTTCAGCTCCAGGAGTCAGGCCCAGGACTCGTTAAACCCTCCGA |
| | GACACTCTCCCTGACATGCACAGTCTCCGGCTACTCCATCACATCAG |
| | ACTCCGCCTGGAACTGGATCAGACAGCCCCCAGGAAAGGGGCTCGA |
| | ATGGATTGGCTATATCTCCTACTCCGGCTCTACCTCCTACAACCCCTC |
| | CCTGAAGTCCCGCGTCACCATCTCTCGCGACACCTCTAAGAACCAGT |
| | TCTCCCTCAAACTGTCCTCCGTGACCGCCGCTGACACCGCTGTTTACT |
| | ACTGCGTGCGCGGCCTGAGATTTGCCTACTGGGGACAGGGCACCCTC |
| | GTGACAGTGTCTAGC (SEQ ID NO: 81) |
| VH | CAGGTTCAGCTCCAGGAGTCAGGCCCAGGACTCGTTAAACCCTCCGA |
| + | GACACTCTCCCTGACATGCACAGTCTCCGGCTACTCCATCACATCAG |
| CH | ACTCCGCCTGGAACTGGATCAGACAGCCCCCAGGAAAGGGGCTCGA |
| | ATGGATTGGCTATATCTCCTACTCCGGCTCTACCTCCTACAACCCCTC |
| | CCTGAAGTCCCGCGTCACCATCTCTCGCGACACCTCTAAGAACCAGT |
| | TCTCCCTCAAACTGTCCTCCGTGACCGCCGCTGACACCGCTGTTTACT |
| | ACTGCGTGCGCGGCCTGAGATTTGCCTACTGGGGACAGGGCACCCTC |
| | GTGACAGTGTCTAGCGCTAGCACCAAGGGCCCATCGGTCTTCCCCCT |
| | GGCACCCTCCTCCAAGAGCACCTCTGGGGGCACAGCGGCCCTGGGCT |
| | GCCTGGTCAAGGACTACTTCCCCGAACCGGTGACGGTGTCGTGGAAC |
| | TCAGGCGCCCTGACCAGCGGCGTGCACACCTTCCCGGCTGTCCTACA |
| | GTCCTCAGGACTCTACTCCCTCAGCAGCGTGGTGACCGTGCCCTCCA |
| | GCAGCTTGGGCACCCAGACCTACATCTGCAACGTGAATCACAAGCCC |
| | AGCAACACCAAGGTGGACAAGAAAGTTGAGCCCAAATCTTGTGACA |
| | AAACTCACACATGCCCACCGTGCCCAGCACCTGAACTCCTGGGGGGA |
| | CCGTCAGTCTTCCTCTTCCCCCCAAAACCCAAGGACACCCTCATGATC |
| | TCCCGGACCCCCGAGGTCACATGCGTGGTGGTGGACGTGAGCCACGA |
| | AGACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCC |
| | ATCGAGAAAACCATCTCCAAAGCCAAAGGGCAGCCCCGAGAACCAC |
| | AGGTGTACACCCTGCCCCCATCCCGGGAGGAGATGACCAAGAACCA |
| | GGTCAGCCTGACCTGCCTGGTCAAAGGCTTCTATCCCAGCGACATCG |
| | CCGTGGAGTGGGAGAGCAATGGGCAGCCGGAGAACAACTACAAGAC |
| | CACGCCTCCCGTGCTGGACTCCGACGGCTCCTTCTTCCTCTACAGCAA |
| | GCTCACCGTGGACAAGAGCAGGTGGCAGCAGGGGAACGTCTTCTCAT |
| | GCTCCGTGATGCATGAGGCTCTGCACAACCACTACACGCAGAAGAGC |
| | CTCTCCCTGTCTCCGGGTAAA (SEQ ID NO: 82) |
| VL | GAAATCGTGCTGACCCAGTCCCCCGCTACCTTGAGTCTGTCCCCTGGA |
| | GAGAGAGCCACCCTGTCTTGTAGGGCTAGTGAGAGCGTGGACTCCTA |
| | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | G (SEQ ID NO: 83) |
| VL | GAAATCGTGCTGACCCAGTCCCCCGCTACCTTGAGTCTGTCCCCTGGA |
| + | GAGAGAGCCACCCTGTCTTGTAGGGCTAGTGAGAGCGTGGACTCCTA |
| CL | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 84) |

Antibody No. D6DD3D006 (HU1127-hVH5/hVL1)

| | |
|---|---|
| VH | CAGGTTCAGCTCCAGGAGTCAGGCCCAGGACTCGTTAAACCCTCCCA |
| | GACCCTCTCCCTCACCTGCACAGTCTCCGGTTACTCCATCACCTCCGA |
| | CAGCGCATGGAACTGGATCAGACAGCCCCCCGGAAAAGGCCTCGAA |
| | TGGATTGGCTATATCTCCTACTCCGGCTCCACCTCCTACAACCCCTCC |
| | TTGAAGTCCCGCGTGACCATCAGCCGGGACACATCAAAGAACCAGTT |
| | CTCCCTCAAGCTGTCTAGCGTGACTGCCGCCGACACCGCTGTTTACTA |
| | CTGCGTGCGGGGCCTGAGATTTGCCTACTGGGGACAGGGCACCCTGG |
| | TGACAGTGTCTAGC (SEQ ID NO: 85) |
| VH | CAGGTTCAGCTCCAGGAGTCAGGCCCAGGACTCGTTAAACCCTCCCA |
| + | GACCCTCTCCCTCACCTGCACAGTCTCCGGTTACTCCATCACCTCCGA |
| CH | CAGCGCATGGAACTGGATCAGACAGCCCCCCGGAAAAGGCCTCGAA |
| | TGGATTGGCTATATCTCCTACTCCGGCTCCACCTCCTACAACCCCTCC |
| | TTGAAGTCCCGCGTGACCATCAGCCGGGACACATCAAAGAACCAGTT |
| | CTCCCTCAAGCTGTCTAGCGTGACTGCCGCCGACACCGCTGTTTACTA |
| | CTGCGTGCGGGGCCTGAGATTTGCCTACTGGGGACAGGGCACCCTGG |
| | TGACAGTGTCTAGCGCTAGCACCAAGGGCCCATCGGTCTTCCCCCTG |
| | GCACCCTCCTCCAAGAGCACCTCTGGGGGCACAGCGGCCCTGGGCTG |
| | CCTGGTCAAGGACTACTTCCCCGAACCGGTGACGGTGTCGTGGAACT |
| | CAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 86) |
| VL | GAAATCGTGCTGACCCAGTCCCCCGCTACCTTGAGTCTGTCCCCTGGA |
| | GAGAGAGCCACCCTGTCTTGTAGGGCTAGTGAGAGCGTGGACTCCTA |
| | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | G (SEQ ID NO: 87) |
| VL | GAAATCGTGCTGACCCAGTCCCCCGCTACCTTGAGTCTGTCCCCTGGA |
| + | GAGAGAGCCACCCTGTCTTGTAGGGCTAGTGAGAGCGTGGACTCCTA |
| CL | CGTGAACTCCTTTCTGCACTGGTACCAGCAGAAGCCAGGCCAGGCTC |
| | CTCGATTGTTGATCTACAGAGCCAGCAACTTGCAGTCCGGGATTCCT |
| | GCCAGGTTTTCCGGCTCCGGTTCCAGAACCGACTTTACCCTCACCATC |
| | TCCTCCCTGGAGCCCGAAGATGTCGCCGTTTACTACTGCCAGCAGTCC |
| | AACGAGGACCCCTACACCTTCGGTCAGGGCACTAAGCTCGAAATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 88) |

Antibody No. D6DD3D007 (HU1127-hVH1/hVL2)

| | |
|---|---|
| VH | CAGGTTCAGCTCCAGGAGTCAGGCCCAGGACTCGTTAAACCCTCCCA |
| | GACCCTCTCCCTCACCTGCACAGTCTCCGGTGGAAGCATCTCCTCCGA |
| | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCC (SEQ ID NO: 89) |
| VH | CAGGTTCAGCTCCAGGAGTCAGGCCCAGGACTCGTTAAACCCTCCCA |
| + | GACCCTCTCCCTCACCTGCACAGTCTCCGGTGGAAGCATCTCCTCCGA |
| CH | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCCGCTAGCACCAAGGGCCCATCGGTCTTCCCCCTGG |
| | CACCCTCCTCCAAGAGCACCTCTGGGGGCACAGCGGCCCTGGGCTGC |
| | CTGGTCAAGGACTACTTCCCCGAACCGGTGACGGTGTCGTGGAACTC |
| | AGGCGCCCTGACCAGCGGCGTGCACACCTTCCCGGCTGTCCTACAGT |
| | CCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 90) |
| VL | GATATCGTGCTGACCCAGTCCCCCGACTCCTTGGCTGTTTCCCTGGGA |
| | GAGAGGGCCACCATCAACTGTAGGGCCTCCGAGTCCGTGGACTCCTA |
| | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | G (SEQ ID NO: 91) |
| VL | GATATCGTGCTGACCCAGTCCCCCGACTCCTTGGCTGTTTCCCTGGGA |
| + | GAGAGGGCCACCATCAACTGTAGGGCCTCCGAGTCCGTGGACTCCTA |
| CL | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 92) |

Antibody No. D6DD3D008 (HU1127-hVH2/hVL2)

| | |
|---|---|
| VH | CAGGTTCAGCTCCAGGAGTCAGGCCCAGGACTCGTTAAACCCTCCGA |
| | GACACTCTCCCTGACATGCACAGTCTCCGGCGGAAGCATCTCCTCCG |
| | ACTCAGCTTGGAACTGGATCAGGCAGCCCCCCGGAAAGGGTTTGGAG |
| | TGGATTGGCTATATCTCCTACTCCGGCTCCACCTCCTACAACCCAAGC |
| | CTGAAAAGCCGAGTGACCATCTCTAGGGACACCTCCAAGAACCAGTT |
| | CTCCCTCAAACTGAGCAGCGTGACCGCCGCTGACACCGCTGTTTACT |
| | ACTGCGTCCGCGGCCTGAGGTTCGCTTACTGGGGACAGGGAACCCTC |
| | GTCACCGTGTCTAGC (SEQ ID NO: 93) |
| VH | CAGGTTCAGCTCCAGGAGTCAGGCCCAGGACTCGTTAAACCCTCCGA |
| + | GACACTCTCCCTGACATGCACAGTCTCCGGCGGAAGCATCTCCTCCG |
| CH | ACTCAGCTTGGAACTGGATCAGGCAGCCCCCCGGAAAGGGTTTGGAG |
| | TGGATTGGCTATATCTCCTACTCCGGCTCCACCTCCTACAACCCAAGC |
| | CTGAAAAGCCGAGTGACCATCTCTAGGGACACCTCCAAGAACCAGTT |
| | CTCCCTCAAACTGAGCAGCGTGACCGCCGCTGACACCGCTGTTTACT |
| | ACTGCGTCCGCGGCCTGAGGTTCGCTTACTGGGGACAGGGAACCCTC |
| | GTCACCGTGTCTAGCGCTAGCACCAAGGGCCCATCGGTCTTCCCCCT |
| | GGCACCCTCCTCCAAGAGCACCTCTGGGGGCACAGCGGCCCTGGGCT |
| | GCCTGGTCAAGGACTACTTCCCCGAACCGGTGACGGTGTCGTGGAAC |
| | TCAGGCGCCCTGACCAGCGGCGTGCACACCTTCCCGGCTGTCCTACA |
| | GTCCTCAGGACTCTACTCCCTCAGCAGCGTGGTGACCGTGCCCTCCA |
| | GCAGCTTGGGCACCCAGACCTACATCTGCAACGTGAATCACAAGCCC |
| | AGCAACACCAAGGTGGACAAGAAAGTTGAGCCCAAATCTTGTGACA |
| | AAACTCACACATGCCCACCGTGCCCAGCACCTGAACTCCTGGGGGGA |
| | CCGTCAGTCTTCCTCTTCCCCCCAAAACCCAAGGACACCCTCATGATC |
| | TCCCGGACCCCCGAGGTCACATGCGTGGTGGTGGACGTGAGCCACGA |
| | AGACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCC |
| | ATCGAGAAAACCATCTCCAAAGCCAAAGGGCAGCCCCGAGAACCAC |
| | AGGTGTACACCCTGCCCCCATCCCGGGAGGAGATGACCAAGAACCA |
| | GGTCAGCCTGACCTGCCTGGTCAAAGGCTTCTATCCCAGCGACATCG |
| | CCGTGGAGTGGGAGAGCAATGGGCAGCCGGAGAACAACTACAAGAC |
| | CACGCCTCCCGTGCTGGACTCCGACGGCTCCTTCTTCCTCTACAGCAA |
| | GCTCACCGTGGACAAGAGCAGGTGGCAGCAGGGGAACGTCTTCTCAT |
| | GCTCCGTGATGCATGAGGCTCTGCACAACCACTACACGCAGAAGAGC |
| | CTCTCCCTGTCTCCGGGTAAA (SEQ ID NO: 94) |
| VL | GATATCGTGCTGACCCAGTCCCCCGACTCCTTGGCTGTTTCCCTGGGA |
| | GAGAGGGCCACCATCAACTGTAGGGCCTCCGAGTCCGTGGACTCCTA |
| | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | G (SEQ ID NO: 95) |
| VL | GATATCGTGCTGACCCAGTCCCCCGACTCCTTGGCTGTTTCCCTGGGA |
| + | GAGAGGGCCACCATCAACTGTAGGGCCTCCGAGTCCGTGGACTCCTA |
| CL | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID NO: |
| | 96) |

Antibody No. D6DD3D009 (HU1127-hVH3/hVL2)

| | |
|---|---|
| VH | CAGTTGCAGCTCCAGGAGTCCGGTCCAGGACTCGTTAAACCCTCCGA |
| | GACACTCTCCCTCACCTGCACAGTCTCCGGCGGAAGTATCTCCTCCGA |
| | CTCCGCTTGGAACTGGATCAGGCAGCCTCCCGGTAAGGGGTTGGAGT |
| | GGATCGGATATATCTCCTACAGCGGCTCCACCTCCTACAATCCCAGTC |
| | TGAAGTCCAGAGTGACCATCTCCCGCGACACCTCCAAGAACCAGTTC |
| | TCCCTCAAACTGTCCAGCGTGACCGCCGCAGACACCGCTGTTTACTA |
| | CTGCGTGCGCGGCCTGAGATTTGCCTACTGGGGACAGGGCACCCTCG |
| | TGACAGTGTCTAGC (SEQ ID NO: 97) |
| VH | CAGTTGCAGCTCCAGGAGTCCGGTCCAGGACTCGTTAAACCCTCCGA |
| + | GACACTCTCCCTCACCTGCACAGTCTCCGGCGGAAGTATCTCCTCCGA |
| CH | CTCCGCTTGGAACTGGATCAGGCAGCCTCCCGGTAAGGGGTTGGAGT |
| | GGATCGGATATATCTCCTACAGCGGCTCCACCTCCTACAATCCCAGTC |
| | TGAAGTCCAGAGTGACCATCTCCCGCGACACCTCCAAGAACCAGTTC |
| | TCCCTCAAACTGTCCAGCGTGACCGCCGCAGACACCGCTGTTTACTA |
| | CTGCGTGCGCGGCCTGAGATTTGCCTACTGGGGACAGGGCACCCTCG |
| | TGACAGTGTCTAGCGCTAGCACCAAGGGCCCATCGGTCTTCCCCCTG |
| | GCACCCTCCTCCAAGAGCACCTCTGGGGGCACAGCGGCCCTGGGCTG |
| | CCTGGTCAAGGACTACTTCCCCGAACCGGTGACGGTGTCGTGGAACT |
| | CAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 98) |
| VL | GATATCGTGCTGACCCAGTCCCCCGACTCCTTGGCTGTTTCCCTGGGA |
| | GAGAGGGCCACCATCAACTGTAGGGCCTCCGAGTCCGTGGACTCCTA |
| | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | G (SEQ ID NO: 99) |
| VL | GATATCGTGCTGACCCAGTCCCCCGACTCCTTGGCTGTTTCCCTGGGA |
| + | GAGAGGGCCACCATCAACTGTAGGGCCTCCGAGTCCGTGGACTCCTA |
| CL | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 100) |

Antibody No. D6DD3D010 (HU1127-hVH4/hVL2)

| | |
|---|---|
| VH | CAGGTTCAGCTCCAGGAGTCAGGCCCAGGACTCGTTAAACCCTCCGA |
| | GACACTCTCCCTGACATGCACAGTCTCCGGCTACTCCATCACATCAG |
| | ACTCCGCCTGGAACTGGATCAGACAGCCCCCAGGAAAGGGGCTCGA |
| | ATGGATTGGCTATATCTCCTACTCCGGCTCTACCTCCTACAACCCCTC |
| | CCTGAAGTCCCGCGTCACCATCTCTCGCGACACCTCTAAGAACCAGT |
| | TCTCCCTCAAACTGTCCTCCGTGACCGCCGCTGACACCGCTGTTTACT |
| | ACTGCGTGCGCGGCCTGAGATTTGCCTACTGGGGACAGGGCACCCTC |
| | GTGACAGTGTCTAGC (SEQ ID NO: 101) |
| VH | CAGGTTCAGCTCCAGGAGTCAGGCCCAGGACTCGTTAAACCCTCCGA |
| + | GACACTCTCCCTGACATGCACAGTCTCCGGCTACTCCATCACATCAG |
| CH | ACTCCGCCTGGAACTGGATCAGACAGCCCCCAGGAAAGGGGCTCGA |
| | ATGGATTGGCTATATCTCCTACTCCGGCTCTACCTCCTACAACCCCTC |
| | CCTGAAGTCCCGCGTCACCATCTCTCGCGACACCTCTAAGAACCAGT |
| | TCTCCCTCAAACTGTCCTCCGTGACCGCCGCTGACACCGCTGTTTACT |
| | ACTGCGTGCGCGGCCTGAGATTTGCCTACTGGGGACAGGGCACCCTC |
| | GTGACAGTGTCTAGCGCTAGCACCAAGGGCCCATCGGTCTTCCCCCT |
| | GGCACCCTCCTCCAAGAGCACCTCTGGGGGCACAGCGGCCCTGGGCT |
| | GCCTGGTCAAGGACTACTTCCCCGAACCGGTGACGGTGTCGTGGAAC |
| | TCAGGCGCCCTGACCAGCGGCGTGCACACCTTCCCGGCTGTCCTACA |
| | GTCCTCAGGACTCTACTCCCTCAGCAGCGTGGTGACCGTGCCCTCCA |
| | GCAGCTTGGGCACCCAGACCTACATCTGCAACGTGAATCACAAGCCC |
| | AGCAACACCAAGGTGGACAAGAAAGTTGAGCCCAAATCTTGTGACA |
| | AAACTCACACATGCCCACCGTGCCCAGCACCTGAACTCCTGGGGGGA |
| | CCGTCAGTCTTCCTCTTCCCCCCAAAACCCAAGGACACCCTCATGATC |
| | TCCCGGACCCCCGAGGTCACATGCGTGGTGGTGGACGTGAGCCACGA |
| | AGACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCC |
| | ATCGAGAAAACCATCTCCAAAGCCAAAGGGCAGCCCCGAGAACCAC |
| | AGGTGTACACCCTGCCCCCATCCCGGGAGGAGATGACCAAGAACCA |
| | GGTCAGCCTGACCTGCCTGGTCAAAGGCTTCTATCCCAGCGACATCG |
| | CCGTGGAGTGGGAGAGCAATGGGCAGCCGGAGAACAACTACAAGAC |
| | CACGCCTCCCGTGCTGGACTCCGACGGCTCCTTCTTCCTCTACAGCAA |
| | GCTCACCGTGGACAAGAGCAGGTGGCAGCAGGGGAACGTCTTCTCAT |
| | GCTCCGTGATGCATGAGGCTCTGCACAACCACTACACGCAGAAGAGC |
| | CTCTCCCTGTCTCCGGGTAAA (SEQ ID NO: 102) |
| VL | GATATCGTGCTGACCCAGTCCCCCGACTCCTTGGCTGTTTCCCTGGGA |
| | GAGAGGGCCACCATCAACTGTAGGGCCTCCGAGTCCGTGGACTCCTA |
| | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | G (SEQ ID NO: 103) |
| VL | GATATCGTGCTGACCCAGTCCCCCGACTCCTTGGCTGTTTCCCTGGGA |
| + | GAGAGGGCCACCATCAACTGTAGGGCCTCCGAGTCCGTGGACTCCTA |
| CL | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 104) |

Antibody No. D6DD3D011 (HU1127-hVH5/hVL2)

| | |
|---|---|
| VH | CAGGTTCAGCTCCAGGAGTCAGGCCCAGGACTCGTTAAACCCTCCCA |
| | GACCCTCTCCCTCACCTGCACAGTCTCCGGTTACTCCATCACCTCCGA |
| | CAGCGCATGGAACTGGATCAGACAGCCCCCCGGAAAAGGCCTCGAA |
| | TGGATTGGCTATATCTCCTACTCCGGCTCCACCTCCTACAACCCCTCC |
| | TTGAAGTCCCGCGTGACCATCAGCCGGGACACATCAAAGAACCAGTT |
| | CTCCCTCAAGCTGTCTAGCGTGACTGCCGCCGACACCGCTGTTTACTA |
| | CTGCGTGCGGGGCCTGAGATTTGCCTACTGGGGACAGGGCACCCTGG |
| | TGACAGTGTCTAGC (SEQ ID NO: 105) |
| VH | CAGGTTCAGCTCCAGGAGTCAGGCCCAGGACTCGTTAAACCCTCCCA |
| + | GACCCTCTCCCTCACCTGCACAGTCTCCGGTTACTCCATCACCTCCGA |
| CH | CAGCGCATGGAACTGGATCAGACAGCCCCCCGGAAAAGGCCTCGAA |
| | TGGATTGGCTATATCTCCTACTCCGGCTCCACCTCCTACAACCCCTCC |
| | TTGAAGTCCCGCGTGACCATCAGCCGGGACACATCAAAGAACCAGTT |
| | CTCCCTCAAGCTGTCTAGCGTGACTGCCGCCGACACCGCTGTTTACTA |
| | CTGCGTGCGGGGCCTGAGATTTGCCTACTGGGGACAGGGCACCCTGG |
| | TGACAGTGTCTAGCGCTAGCACCAAGGGCCCATCGGTCTTCCCCCTG |
| | GCACCCTCCTCCAAGAGCACCTCTGGGGGCACAGCGGCCCTGGGCTG |
| | CCTGGTCAAGGACTACTTCCCCGAACCGGTGACGGTGTCGTGGAACT |
| | CAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 106) |
| VL | CAGGTTCAGCTCCAGGAGTCAGGCCCAGGACTCGTTAAACCCTCCCA |
| | GACCCTCTCCCTCACCTGCACAGTCTCCGGTGGAAGCATCTCCTCCGA |
| | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCC (SEQ ID NO: 107) |
| VL | GATATCGTGCTGACCCAGTCCCCCGACTCCTTGGCTGTTTCCCTGGGA |
| + | GAGAGGGCCACCATCAACTGTAGGGCCTCCGAGTCCGTGGACTCCTA |
| CL | CGTTAACAGCTTTCTGCATTGGTACCAGCAGAAGCCCGGCCAGCCTC |
| | CTAAGTTGCTCATTTACAGGGCTTCCAACCTCCAGTCCGGCGTGCCTG |
| | ATCGTTTCTCTGGCTCCGGTTCCCGCACCGATTTCACCTTGACCATCT |
| | CCTCCCTGCAGGCTGAGGACGTCGCTGTTTACTACTGCCAGCAGTCC |
| | AACGAAGATCCTTATACCTTTGGACAGGGCACCAAACTCGAAATTAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 108) |

Antibody No. D6DD3D012 (HU1127-hVH1/hVL3)

| | |
|---|---|
| VH | CAGGTTCAGCTCCAGGAGTCAGGCCCAGGACTCGTTAAACCCTCCCA |
| | GACCCTCTCCCTCACCTGCACAGTCTCCGGTGGAAGCATCTCCTCCGA |
| | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCC (SEQ ID NO: 109) |
| VH | CAGGTTCAGCTCCAGGAGTCAGGCCCAGGACTCGTTAAACCCTCCCA |
| + | GACCCTCTCCCTCACCTGCACAGTCTCCGGTGGAAGCATCTCCTCCGA |
| CH | CTCCGCATGGAACTGGATCAGACAGCACCCCGGCAAAGGCCTCGAAT |
| | GGATTGGCTATATCTCCTACTCCGGCTCCACTTCCTACAATCCCTCCC |
| | TCAAGTCCAGAGTGACCATCTCCCGCGACACCTCAAAGAACCAGTTC |
| | TCCCTCAAACTGAGCAGCGTGACCGCCGCTGACACCGCTGTTTACTA |
| | CTGCGTTCGTGGCCTCCGCTTCGCCTACTGGGGACAGGGAACACTCG |
| | TGACCGTGTCCTCCGCTAGCACCAAGGGCCCATCGGTCTTCCCCCTGG |
| | CACCCTCCTCCAAGAGCACCTCTGGGGGCACAGCGGCCCTGGGCTGC |
| | CTGGTCAAGGACTACTTCCCCGAACCGGTGACGGTGTCGTGGAACTC |
| | AGGCGCCCTGACCAGCGGCGTGCACACCTTCCCGGCTGTCCTACAGT |
| | CCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 110) |
| VL | GATATCCAGCTGACCCAGAGCCCCTCCTCCTTGTCTGCATCCGTGGGA |
| | GACAGAGTGACCATTACCTGCCGGGCCTCCGAGTCTGTGGACTCTTA |
| | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | G (SEQ ID NO: 111) |
| VL | GATATCCAGCTGACCCAGAGCCCCTCCTCCTTGTCTGCATCCGTGGGA |
| + | GACAGAGTGACCATTACCTGCCGGGCCTCCGAGTCTGTGGACTCTTA |
| CL | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 112) |

Antibody No. D6DD3D013 (HU1127-hVH2/hVL3

| | |
|---|---|
| VH | CAGGTTCAGCTCCAGGAGTCAGGCCCAGGACTCGTTAAACCCTCCGA |
| | GACACTCTCCCTGACATGCACAGTCTCCGGCGGAAGCATCTCCTCCG |
| | ACTCAGCTTGGAACTGGATCAGGCAGCCCCCCGGAAAGGGTTTGGAG |
| | TGGATTGGCTATATCTCCTACTCCGGCTCCACCTCCTACAACCCAAGC |
| | CTGAAAAGCCGAGTGACCATCTCTAGGGACACCTCCAAGAACCAGTT |
| | CTCCCTCAAACTGAGCAGCGTGACCGCCGCTGACACCGCTGTTTACT |
| | ACTGCGTCCGCGGCCTGAGGTTCGCTTACTGGGGACAGGGAACCCTC |
| | GTCACCGTGTCTAGC (SEQ ID NO: 113) |
| VH | CAGGTTCAGCTCCAGGAGTCAGGCCCAGGACTCGTTAAACCCTCCGA |
| + | GACACTCTCCCTGACATGCACAGTCTCCGGCGGAAGCATCTCCTCCG |
| CH | ACTCAGCTTGGAACTGGATCAGGCAGCCCCCCGGAAAGGGTTTGGAG |
| | TGGATTGGCTATATCTCCTACTCCGGCTCCACCTCCTACAACCCAAGC |
| | CTGAAAAGCCGAGTGACCATCTCTAGGGACACCTCCAAGAACCAGTT |
| | CTCCCTCAAACTGAGCAGCGTGACCGCCGCTGACACCGCTGTTTACT |
| | ACTGCGTCCGCGGCCTGAGGTTCGCTTACTGGGGACAGGGAACCCTC |
| | GTCACCGTGTCTAGCGCTAGCACCAAGGGCCCATCGGTCTTCCCCCT |
| | GGCACCCTCCTCCAAGAGCACCTCTGGGGGCACAGCGGCCCTGGGCT |
| | GCCTGGTCAAGGACTACTTCCCCGAACCGGTGACGGTGTCGTGGAAC |
| | TCAGGCGCCCTGACCAGCGGCGTGCACACCTTCCCGGCTGTCCTACA |
| | GTCCTCAGGACTCTACTCCCTCAGCAGCGTGGTGACCGTGCCCTCCA |
| | GCAGCTTGGGCACCCAGACCTACATCTGCAACGTGAATCACAAGCCC |
| | AGCAACACCAAGGTGGACAAGAAAGTTGAGCCCAAATCTTGTGACA |
| | AAACTCACACATGCCCACCGTGCCCAGCACCTGAACTCCTGGGGGGA |
| | CCGTCAGTCTTCCTCTTCCCCCCAAAACCCAAGGACACCCTCATGATC |
| | TCCCGGACCCCCGAGGTCACATGCGTGGTGGTGGACGTGAGCCACGA |
| | AGACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCC |
| | ATCGAGAAAACCATCTCCAAAGCCAAAGGGCAGCCCCGAGAACCAC |
| | AGGTGTACACCCTGCCCCCATCCCGGGAGGAGATGACCAAGAACCA |
| | GGTCAGCCTGACCTGCCTGGTCAAAGGCTTCTATCCCAGCGACATCG |
| | CCGTGGAGTGGGAGAGCAATGGGCAGCCGGAGAACAACTACAAGAC |
| | CACGCCTCCCGTGCTGGACTCCGACGGCTCCTTCTTCCTCTACAGCAA |
| | GCTCACCGTGGACAAGAGCAGGTGGCAGCAGGGGAACGTCTTCTCAT |
| | GCTCCGTGATGCATGAGGCTCTGCACAACCACTACACGCAGAAGAGC |
| | CTCTCCCTGTCTCCGGGTAAA (SEQ ID NO: 114) |
| VL | GATATCCAGCTGACCCAGAGCCCCTCCTCCTTGTCTGCATCCGTGGGA |
| | GACAGAGTGACCATTACCTGCCGGGCCTCCGAGTCTGTGGACTCTTA |
| | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | G (SEQ ID NO: 115) |
| VL | GATATCCAGCTGACCCAGAGCCCCTCCTCCTTGTCTGCATCCGTGGGA |
| + | GACAGAGTGACCATTACCTGCCGGGCCTCCGAGTCTGTGGACTCTTA |
| CL | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 116) |

Antibody No. D6DD3D014 (HU1127-hVH3/hVL3)

| | |
|---|---|
| VH | CAGTTGCAGCTCCAGGAGTCCGGTCCAGGACTCGTTAAACCCTCCGA |
| | GACACTCTCCCTCACCTGCACAGTCTCCGGCGGAAGTATCTCCTCCGA |
| | CTCCGCTTGGAACTGGATCAGGCAGCCTCCCGGTAAGGGGTTGGAGT |
| | GGATCGGATATATCTCCTACAGCGGCTCCACCTCCTACAATCCCAGTC |
| | TGAAGTCCAGAGTGACCATCTCCCGCGACACCTCCAAGAACCAGTTC |
| | TCCCTCAAACTGTCCAGCGTGACCGCCGCAGACACCGCTGTTTACTA |
| | CTGCGTGCGCGGCCTGAGATTTGCCTACTGGGGACAGGGCACCCTCG |
| | TGACAGTGTCTAGC (SEQ ID NO: 117) |
| VH | CAGTTGCAGCTCCAGGAGTCCGGTCCAGGACTCGTTAAACCCTCCGA |
| + | GACACTCTCCCTCACCTGCACAGTCTCCGGCGGAAGTATCTCCTCCGA |
| CH | CTCCGCTTGGAACTGGATCAGGCAGCCTCCCGGTAAGGGGTTGGAGT |
| | GGATCGGATATATCTCCTACAGCGGCTCCACCTCCTACAATCCCAGTC |
| | TGAAGTCCAGAGTGACCATCTCCCGCGACACCTCCAAGAACCAGTTC |
| | TCCCTCAAACTGTCCAGCGTGACCGCCGCAGACACCGCTGTTTACTA |
| | CTGCGTGCGCGGCCTGAGATTTGCCTACTGGGGACAGGGCACCCTCG |
| | TGACAGTGTCTAGCGCTAGCACCAAGGGCCCATCGGTCTTCCCCCTG |
| | GCACCCTCCTCCAAGAGCACCTCTGGGGGCACAGCGGCCCTGGGCTG |
| | CCTGGTCAAGGACTACTTCCCCGAACCGGTGACGGTGTCGTGGAACT |
| | CAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 118) |
| VL | GATATCCAGCTGACCCAGAGCCCCTCCTCCTTGTCTGCATCCGTGGGA |
| | GACAGAGTGACCATTACCTGCCGGGCCTCCGAGTCTGTGGACTCTTA |
| | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | G (SEQ ID NO: 119) |
| VL | GATATCCAGCTGACCCAGAGCCCCTCCTCCTTGTCTGCATCCGTGGGA |
| + | GACAGAGTGACCATTACCTGCCGGGCCTCCGAGTCTGTGGACTCTTA |
| CL | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 120) |

Antibody No. D6DD3D015 (HU1127-hVH4/hVL3)

| | |
|---|---|
| VH | CAGGTTCAGCTCCAGGAGTCAGGCCCAGGACTCGTTAAACCCTCCGA |
| | GACACTCTCCCTGACATGCACAGTCTCCGGCTACTCCATCACATCAG |
| | ACTCCGCCTGGAACTGGATCAGACAGCCCCCAGGAAAGGGGCTCGA |
| | ATGGATTGGCTATATCTCCTACTCCGGCTCTACCTCCTACAACCCCTC |
| | CCTGAAGTCCCGCGTCACCATCTCTCGCGACACCTCTAAGAACCAGT |
| | TCTCCCTCAAACTGTCCTCCGTGACCGCCGCTGACACCGCTGTTTACT |
| | ACTGCGTGCGCGGCCTGAGATTTGCCTACTGGGGACAGGGCACCCTC |
| | GTGACAGTGTCTAGC (SEQ ID NO: 121) |
| VH | CAGGTTCAGCTCCAGGAGTCAGGCCCAGGACTCGTTAAACCCTCCGA |
| + | GACACTCTCCCTGACATGCACAGTCTCCGGCTACTCCATCACATCAG |
| CH | ACTCCGCCTGGAACTGGATCAGACAGCCCCCAGGAAAGGGGCTCGA |
| | ATGGATTGGCTATATCTCCTACTCCGGCTCTACCTCCTACAACCCCTC |
| | CCTGAAGTCCCGCGTCACCATCTCTCGCGACACCTCTAAGAACCAGT |
| | TCTCCCTCAAACTGTCCTCCGTGACCGCCGCTGACACCGCTGTTTACT |
| | ACTGCGTGCGCGGCCTGAGATTTGCCTACTGGGGACAGGGCACCCTC |
| | GTGACAGTGTCTAGCGCTAGCACCAAGGGCCCATCGGTCTTCCCCCT |
| | GGCACCCTCCTCCAAGAGCACCTCTGGGGGCACAGCGGCCCTGGGCT |
| | GCCTGGTCAAGGACTACTTCCCCGAACCGGTGACGGTGTCGTGGAAC |
| | TCAGGCGCCCTGACCAGCGGCGTGCACACCTTCCCGGCTGTCCTACA |
| | GTCCTCAGGACTCTACTCCCTCAGCAGCGTGGTGACCGTGCCCTCCA |
| | GCAGCTTGGGCACCCAGACCTACATCTGCAACGTGAATCACAAGCCC |
| | AGCAACACCAAGGTGGACAAGAAAGTTGAGCCCAAATCTTGTGACA |
| | AAACTCACACATGCCCACCGTGCCCAGCACCTGAACTCCTGGGGGGA |
| | CCGTCAGTCTTCCTCTTCCCCCCAAAACCCAAGGACACCCTCATGATC |
| | TCCCGGACCCCCGAGGTCACATGCGTGGTGGTGGACGTGAGCCACGA |
| | AGACCCTGAGGTCAAGTTCAACTGGTACGTGGACGGCGTGGAGGTGC |
| | ATAATGCCAAGACAAAGCCGCGGGAGGAGCAGTACAACAGCACGTA |
| | CCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCC |
| | ATCGAGAAAACCATCTCCAAAGCCAAAGGGCAGCCCCGAGAACCAC |
| | AGGTGTACACCCTGCCCCCATCCCGGGAGGAGATGACCAAGAACCA |
| | GGTCAGCCTGACCTGCCTGGTCAAAGGCTTCTATCCCAGCGACATCG |
| | CCGTGGAGTGGGAGAGCAATGGGCAGCCGGAGAACAACTACAAGAC |
| | CACGCCTCCCGTGCTGGACTCCGACGGCTCCTTCTTCCTCTACAGCAA |
| | GCTCACCGTGGACAAGAGCAGGTGGCAGCAGGGGAACGTCTTCTCAT |
| | GCTCCGTGATGCATGAGGCTCTGCACAACCACTACACGCAGAAGAGC |
| | CTCTCCCTGTCTCCGGGTAAA (SEQ ID NO: 122) |
| VL | GATATCCAGCTGACCCAGAGCCCCTCCTCCTTGTCTGCATCCGTGGGA |
| | GACAGAGTGACCATTACCTGCCGGGCCTCCGAGTCTGTGGACTCTTA |
| | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | G (SEQ ID NO: 123) |
| VL | GATATCCAGCTGACCCAGAGCCCCTCCTCCTTGTCTGCATCCGTGGGA |
| + | GACAGAGTGACCATTACCTGCCGGGCCTCCGAGTCTGTGGACTCTTA |
| CL | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 124) |

Antibody No. D6DD3D016 (HU1127-hVH5/hVL3)

| | |
|---|---|
| VH | CAGGTTCAGCTCCAGGAGTCAGGCCCAGGACTCGTTAAACCCTCCCA |
| | GACCCTCTCCCTCACCTGCACAGTCTCCGGTTACTCCATCACCTCCGA |
| | CAGCGCATGGAACTGGATCAGACAGCCCCCCGGAAAAGGCCTCGAA |
| | TGGATTGGCTATATCTCCTACTCCGGCTCCACCTCCTACAACCCCTCC |
| | TTGAAGTCCCGCGTGACCATCAGCCGGGACACATCAAAGAACCAGTT |
| | CTCCCTCAAGCTGTCTAGCGTGACTGCCGCCGACACCGCTGTTTACTA |
| | CTGCGTGCGGGGCCTGAGATTTGCCTACTGGGGACAGGGCACCCTGG |
| | TGACAGTGTCTAGC (SEQ ID NO: 125) |
| VH | CAGGTTCAGCTCCAGGAGTCAGGCCCAGGACTCGTTAAACCCTCCCA |
| + | GACCCTCTCCCTCACCTGCACAGTCTCCGGTTACTCCATCACCTCCGA |
| CH | CAGCGCATGGAACTGGATCAGACAGCCCCCCGGAAAAGGCCTCGAA |
| | TGGATTGGCTATATCTCCTACTCCGGCTCCACCTCCTACAACCCCTCC |
| | TTGAAGTCCCGCGTGACCATCAGCCGGGACACATCAAAGAACCAGTT |
| | CTCCCTCAAGCTGTCTAGCGTGACTGCCGCCGACACCGCTGTTTACTA |
| | CTGCGTGCGGGGCCTGAGATTTGCCTACTGGGGACAGGGCACCCTGG |
| | TGACAGTGTCTAGCGCTAGCACCAAGGGCCCATCGGTCTTCCCCCTG |
| | GCACCCTCCTCCAAGAGCACCTCTGGGGGCACAGCGGCCCTGGGCTG |
| | CCTGGTCAAGGACTACTTCCCCGAACCGGTGACGGTGTCGTGGAACT |
| | CAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGC |
| | AGCTTGGGCACCCAGACCTACATCTGCAACGTGAATCACAAGCCCAG |
| | CAACACCAAGGTGGACAAGAAAGTTGAGCCCAAATCTTGTGACAAA |
| | ACTCACACATGCCCACCGTGCCCAGCACCTGAACTCCTGGGGGGACC |
| | GTCAGTCTTCCTCTTCCCCCCAAAACCCAAGGACACCCTCATGATCTC |
| | CCGGACCCCCGAGGTCACATGCGTGGTGGTGGACGTGAGCCACGAA |
| | GACCCTGAGGTCAAGTTCAACTGGTACGTGGACGGCGTGGAGGTGCA |
| | TAATGCCAAGACAAAGCCGCGGGAGGAGCAGTACAACAGCACGTAC |
| | CGTGTGGTCAGCGTCCTCACCGTCCTGCACCAGGACTGGCTGAATGG |
| | CAAGGAGTACAAGTGCAAGGTCTCCAACAAAGCCCTCCCAGCCCCCA |
| | TCGAGAAAACCATCTCCAAAGCCAAAGGGCAGCCCCGAGAACCACA |
| | GGTGTACACCCTGCCCCCATCCCGGGAGGAGATGACCAAGAACCAG |
| | GTCAGCCTGACCTGCCTGGTCAAAGGCTTCTATCCCAGCGACATCGC |
| | CGTGGAGTGGGAGAGCAATGGGCAGCCGGAGAACAACTACAAGACC |
| | ACGCCTCCCGTGCTGGACTCCGACGGCTCCTTCTTCCTCTACAGCAAG |
| | CTCACCGTGGACAAGAGCAGGTGGCAGCAGGGGAACGTCTTCTCATG |
| | CTCCGTGATGCATGAGGCTCTGCACAACCACTACACGCAGAAGAGCC |
| | TCTCCCTGTCTCCGGGTAAA (SEQ ID NO: 126) |
| VL | GATATCCAGCTGACCCAGAGCCCCTCCTCCTTGTCTGCATCCGTGGGA |
| | GACAGAGTGACCATTACCTGCCGGGCCTCCGAGTCTGTGGACTCTTA |
| | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | G (SEQ ID NO: 127) |
| VL | GATATCCAGCTGACCCAGAGCCCCTCCTCCTTGTCTGCATCCGTGGGA |
| + | GACAGAGTGACCATTACCTGCCGGGCCTCCGAGTCTGTGGACTCTTA |
| CL | CGTGAACAGCTTCTTGCACTGGTACCAGCAGAAACCCGGCAAAGCCC |
| | CCAAGCTGCTCATTTACAGGGCCTCCAACCTGCAGTCCGGCGTTCCTT |
| | CTCGCTTTTCCGGGTCCGGTTCCAGGACCGATTTCACCCTCACCATCT |
| | CCTCCCTGCAGCCCGAAGATGTTGCTACCTACTACTGCCAGCAGTCC |
| | AACGAGGACCCCTACACCTTCGGCCAGGGTACTAAGCTGGAGATCAA |
| | GCGTACGGTGGCTGCACCATCTGTCTTCATCTTCCCGCCATCTGATGA |
| | GCAGTTGAAATCTGGAACTGCCTCTGTTGTGTGCCTGCTGAATAACTT |
| | CTATCCCAGAGAGGCCAAAGTACAGTGGAAGGTGGATAACGCCCTCC |
| | AATCGGGTAACTCCCAGGAGAGTGTCACAGAGCAGGACAGCAAGGA |
| | CAGCACCTACAGCCTCAGCAGCACCCTGACGCTGAGCAAAGCAGACT |
| | ACGAGAAACACAAAGTCTACGCCTGCGAAGTCACCCATCAGGGCCTG |
| | AGTTCGCCCGTCACAAAGAGCTTCAACAGGGGAGAGTGT (SEQ ID |
| | NO: 128) |

Nucleotide sequences shown in Table 5-4 may include the following nucleotide sequence at the 5′ end:

(SEQ ID NO: 156)
GCGGCCGCAAACTACAAGACAGACTTGCAAAAGAAGGCATGCACA
GCTCAGCACTGCTCTGTTGCCTGGTCCTCCTGACTGGGGTGAGGGCC.

SEQ ID NO: 156 includes the restriction site for enzyme Not1 (GCGGCCGC).

Nucleotide sequences shown in Table 5-4 may include the following nucleotide sequence at the 3′ end: TGATTCTAGA (SEQ ID NO: 157). SEQ ID NO: 157 includes the restriction site for enzyme Xba1 (TCTAGA)).

### Example 6. Anti-CD14 Monoclonal Antibody Mediated Killing Against Cancer Cell Line Derived Xenograft: IgG1 and IgG4 Efficiency for Tumor Treatment

It was identified that the malignant recurrent bladder cancer from patients still had highly expressed CD14 tumor cells after passaging in NOD mice (FIG. 2).

The efficiency for tumor treatment of the IgG1 version (D6DD3D001) was evaluated. For these experiments, eight (8) treatment groups for PDX mice were set up: control IgG group (C.Ex. 1), single cisplatin group (4 mg/kg; C.Ex. 2), single anti-CD14-IgG1 group (5 mg/kg; Ex. 1), single anti-CD14-IgG4 (5 mg/kg; Ex. 2), combined cisplatin and anti-CD14-IgG1 group (4 mg/kg+1.5 mg/kg; Ex. 3), combined cisplatin and anti-CD14-IgG4 group (4 mg/kg+1.5 mg/kg; Ex. 4), combined cisplatin and anti-CD14-IgG1 group (4 mg/kg+5 mg/kg; Ex. 5), and combined cisplatin and anti-CD14-IgG4 group (4 mg/kg+5 mg/kg; Ex. 6). The IgG4 tested is the original version of Atibuclimab. When cisplatin was used in these experiments, the cisplatin was used at a dose of 4 mg/kg. “C.Ex.” refers to comparative examples and “Ex.” refers to examples of embodiments of the present disclosure. The control is a non-specific IgG1 having two identical heavy chains and two identical light chains. The non-specific IgG1 has no binding affinity to the antigen.

Drug administration on the PDX mice was performed intravenously. Drug administration was performed on days 0, 4, 7, 11, 14, 18, 21, 25, and 28. Measurements of length, width, volume, and weight were performed prior to the drug administration on day 0, 4, 7, 11, 14, 18, 21, 25, and 28. The tumors were present on the skin of the mice. A ruler was used to measure the length and the width. The tumor volume was calculated by volume=½(length×width\hat{ }2). The weight of the mouse was measured by placing the mouse on a scale and weighing. Tumor weight was measured by removing the tumor from the mouse and weighing on a scale.

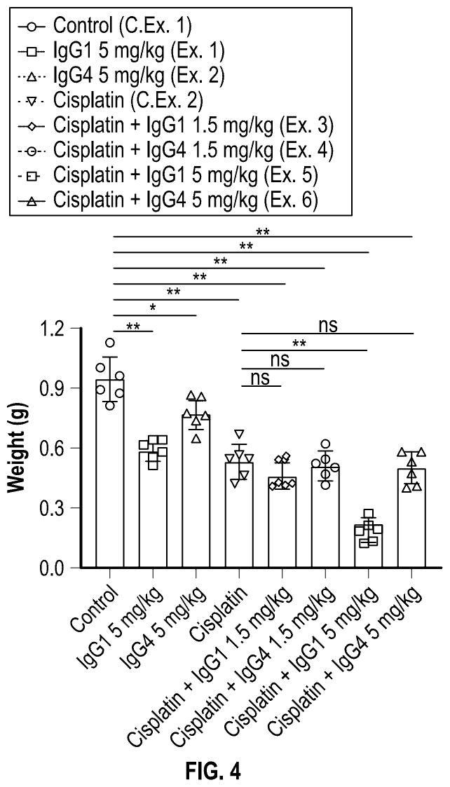

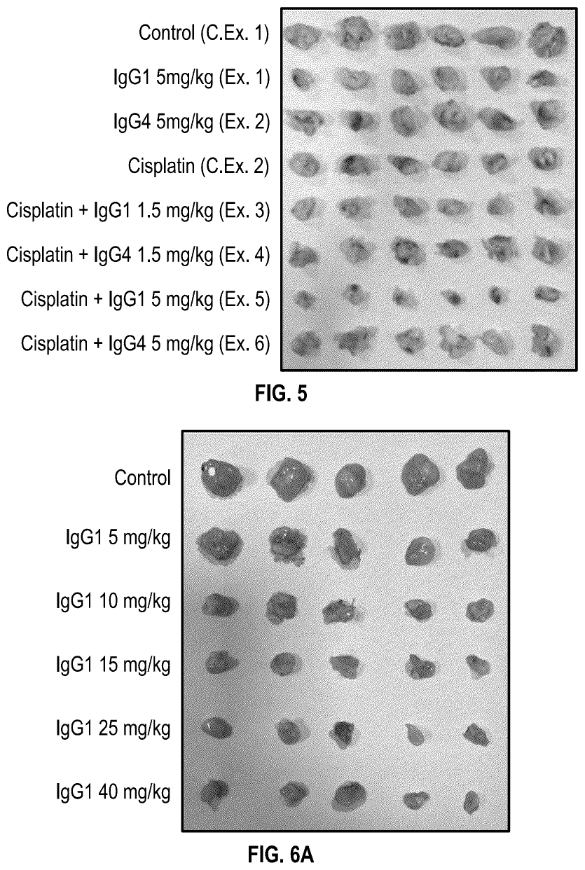

Results of the experiments are shown in FIGS. 3-5. Specifically, FIG. 3 shows exemplary data for tumor volume (mm³) under the different treatment regimens according to at least one embodiment of the present disclosure. FIG. 4 shows exemplary data for tumor weight after 28 days of the different treatment regimens according to at least one embodiment of the present disclosure.

Additionally, tables 6-1 to 6-8 (below) show the weights of the mice under different treatments, indicating that the weights are similar and stable regardless of the dosage (ranging from 5 mg/kg to 40 mg/kg). These findings indicate that there are no safety concerns, even with a 40 mg/kg dosage of the antibody.

All examples outperformed the control regimen (C.Ex. 1). After 28 days of experimentation (drug administration on alternating 4 days and 3 days of day 0, 4, 7, 11, 14, 18, 21, 25, and 28), the single cisplatin group (C.Ex. 2) and the single anti-CD14-IgG1 group (Ex. 1) had similar effects, with a tumor suppression rate of about 50% compared to the control group. The combined cisplatin and anti-CD14-IgG4 (Ex. 4), the combined cisplatin and anti-CD14-IgG1 (Ex. 3), and the combined cisplatin and anti-CD14-IgG4 (Ex. 6) groups had a tumor suppression rate of about 60% after 28 days (FIG. 3 and FIG. 4). The effect of using the low cytotoxicity IgG4 antibody alone was generally not significant, with a tumor suppression rate of about 25% (Ex. 2). The best effect was observed in the combined cisplatin and anti-CD14-IgG1 group (Ex. 5), which provided an 80% tumor suppression rate on day 28. It was also noted that Ex. 3 and Ex. 5 performed better than cisplatin alone. The weight of all mice under the different treatments were similar and stable regardless of drug administered, indicating that there are no safety concerns under the different treatment regimens.

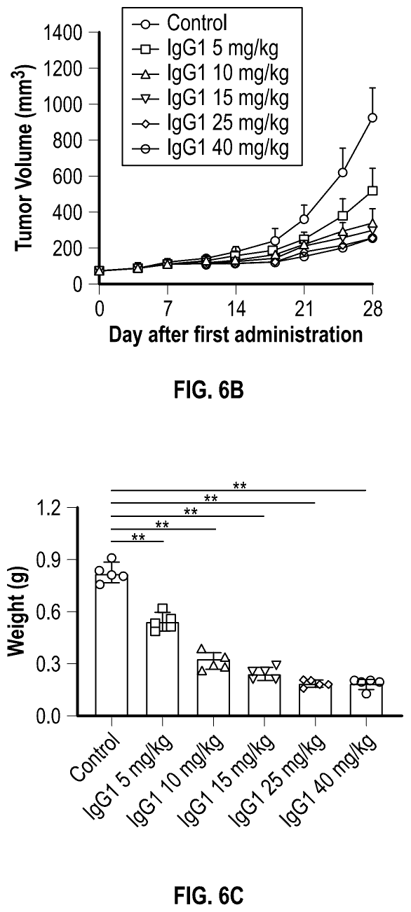

FIG. 5 shows exemplary images of the tumors at 28 days according to at least one embodiment of the present disclosure. The tumors were collected from the PDX mice on day 28 and the weight of the mice were determined. No significant differences in body weight changes between the various treatment groups of mice was found. Overall, the exemplary data indicated that embodiments described herein can have strong cytotoxicity against CD14 positive tumors. The results shown in FIGS. 3-5 confirmed that the use of anti-CD14 antibodies (especially for IgG1 instead of IgG4, with or without combination with cisplatin) had strong cytotoxicity against CD14 positive tumors. Here, the results suggests that ADCC is triggered by specific binding of the example IgG1 antibodies to CD14 positive tumor cells. This demonstrates that the example IgG1 antibodies described herein possess substantial capability of inducing ADCC killing against CD14 positive tumor cells.

Tables 6-1 to 6-8 show selected raw data utilized for FIG. 3. In Tables 6-1 to 6-8, the length, width, and volume shown are the tumor dimensions, and the weight is the mouse weight.

TABLE 6-1
Control Group (C. Ex. 1)

| Number | 1 | 2 | 3 | 4 | 5 | 6 |
|---|---|---|---|---|---|---|
| Day 0 | | | | | | |
| Tumor Length, mm | 5.52 | 7.44 | 6.99 | 7.28 | 6.15 | 6.29 |
| Tumor Width, mm | 4.73 | 4.57 | 5.12 | 5.26 | 4.91 | 6.21 |
| Tumor Volume, mm³ | 64.63 | 81.32 | 95.89 | 105.41 | 77.59 | 126.94 |
| Mouse Weight, g | 20.41 | 20.9 | 19.93 | 20.45 | 20.51 | 20.67 |
| Day 4 | | | | | | |
| Tumor Length, mm | 7.52 | 7.76 | 6.89 | 6.68 | 5.74 | 6.59 |
| Tumor Width, mm | 5.35 | 5.04 | 6.35 | 5.7 | 5.08 | 5.9 |
| Tumor Volume, mm³ | 112.64 | 103.16 | 145.39 | 113.58 | 77.52 | 120.05 |
| Mouse Weight, g | 20.82 | 20.96 | 20.84 | 20.53 | 19.89 | 20.42 |
| Day 7 | | | | | | |
| Tumor Length, mm | 6.15 | 6.66 | 6.57 | 8.37 | 8.94 | 7.17 |
| Tumor Width, mm | 6.06 | 6.35 | 6.35 | 5.95 | 5.66 | 7.08 |
| Tumor Volume, mm³ | 118.19 | 140.54 | 138.64 | 155.07 | 149.88 | 188.09 |
| Mouse Weight, g | 20.91 | 21.5 | 20.73 | 19.51 | 20.17 | 20.4 |
| Day 11 | | | | | | |
| Tumor Length, mm | 8.7 | 7.97 | 7.44 | 7.95 | 8.79 | 7.11 |
| Tumor Width, mm | 7.34 | 6.41 | 7.08 | 6.34 | 8.54 | 6.73 |
| Tumor Volume, mm³ | 245.30 | 171.38 | 195.17 | 167.23 | 335.49 | 168.53 |
| Mouse Weight, g | 21.89 | 20.61 | 20.58 | 21.36 | 21.55 | 19.94 |
| Day 14 | | | | | | |
| Tumor Length, mm | 8.95 | 8.67 | 7.73 | 9.82 | 7.44 | 9.31 |
| Tumor Width, mm | 8.72 | 7.94 | 7.73 | 7.56 | 6.7 | 8.28 |
| Tumor Volume, mm³ | 356.15 | 286.05 | 241.72 | 293.72 | 174.78 | 334.03 |
| Mouse Weight, g | 20.96 | 21.78 | 21.46 | 22.3 | 20.76 | 21.81 |
| Day 18 | | | | | | |
| Tumor Length, mm | 9.84 | 8.93 | 11.13 | 8.62 | 9.52 | 9.97 |
| Tumor Width, mm | 8.36 | 8.71 | 9.18 | 10.08 | 8.84 | 7.88 |
| Tumor Volume, mm³ | 359.90 | 354.54 | 490.86 | 458.36 | 389.33 | 323.99 |
| Mouse Weight, g | 21.88 | 22.07 | 23.11 | 21.34 | 23.2 | 21.27 |
| Day 21 | | | | | | |
| Tumor Length, mm | 9.54 | 9.91 | 10.88 | 10.85 | 11.2 | 9.85 |
| Tumor Width, mm | 9.2 | 9.78 | 10.05 | 10.33 | 10.27 | 9.62 |
| Tumor Volume, mm³ | 422.57 | 496.05 | 575.09 | 605.91 | 618.21 | 477.05 |
| Mouse Weight, g | 23.9 | 23.68 | 21.92 | 22.04 | 23.83 | 22.72 |
| Day 25 | | | | | | |
| Tumor Length, mm | 12.97 | 11.47 | 11.08 | 12.59 | 11.89 | 12.23 |
| Tumor Width, mm | 11.5 | 10.06 | 9.96 | 10.69 | 10.94 | 11.97 |
| Tumor Volume, mm³ | 897.66 | 607.49 | 575.22 | 752.94 | 744.72 | 917.05 |
| Mouse Weight, g | 22.92 | 24.22 | 23.42 | 24.24 | 23.73 | 25.58 |
| Day 28 | | | | | | |
| Tumor Length, mm | 12.91 | 13.75 | 14.24 | 14.46 | 13.54 | 12.88 |
| Tumor Width, mm | 11.36 | 12.27 | 13.09 | 13.14 | 12.3 | 11.81 |
| Tumor Volume, mm³ | 871.89 | 1083.35 | 1276.93 | 1306.58 | 1072.03 | 940.14 |
| Mouse Weight, g | 26 | 25.07 | 25.86 | 26.24 | 25.9 | 24.66 |

TABLE 6-2
IgG1 (5 mg/kg) Group (Ex. 1)

| Number | 1 | 2 | 3 | 4 | 5 | 6 |
|---|---|---|---|---|---|---|
| Day 0 | | | | | | |
| Tumor Length, mm | 5.49 | 6.47 | 6.35 | 7.09 | 6.67 | 6.45 |
| Tumor Width, mm | 4.68 | 5.9 | 5.17 | 4.77 | 5.25 | 5.24 |
| Tumor Volume, mm³ | 62.93 | 117.87 | 88.82 | 84.42 | 96.21 | 92.68 |
| Mouse Weight, g | 20.87 | 20.55 | 20.17 | 20.26 | 20.09 | 20.14 |
| Day 4 | | | | | | |
| Tumor Length, mm | 7.07 | 6.57 | 5.62 | 7.08 | 6.02 | 6.24 |
| Tumor Width, mm | 5.98 | 5.95 | 5.01 | 5.53 | 5.58 | 6.18 |
| Tumor Volume, mm³ | 132.31 | 121.72 | 73.82 | 113.31 | 98.09 | 124.72 |
| Mouse Weight, g | 20.88 | 20.87 | 20.24 | 21 | 20.73 | 20.04 |
| Day 7 | | | | | | |
| Tumor Length, mm | 7.09 | 6.9 | 6.93 | 6.25 | 6.83 | 8.42 |
| Tumor Width, mm | 5.61 | 6.26 | 6.41 | 5.39 | 6.72 | 6.39 |
| Tumor Volume, mm³ | 116.78 | 141.51 | 149.01 | 95.02 | 161.41 | 179.93 |
| Mouse Weight, g | 20.22 | 19.91 | 19.71 | 20.83 | 20.65 | 21.49 |
| Day 11 | | | | | | |
| Tumor Length, mm | 7.21 | 8.38 | 8.42 | 8.88 | 7.93 | 6.52 |
| Tumor Width, mm | 5.85 | 6.91 | 7.5 | 7.03 | 6.5 | 5.39 |
| Tumor Volume, mm³ | 129.13 | 209.40 | 247.86 | 229.67 | 175.34 | 99.13 |
| Mouse Weight, g | 21.66 | 21.77 | 21.54 | 21.15 | 19.96 | 21.01 |
| Day 14 | | | | | | |
| Tumor Length, mm | 8.71 | 8.88 | 8.53 | 8.28 | 9.34 | 8.3 |
| Tumor Width, mm | 7.05 | 6.51 | 7.95 | 7.99 | 8.34 | 8.05 |
| Tumor Volume, mm³ | 226.56 | 196.95 | 282.14 | 276.63 | 339.98 | 281.48 |
| Mouse Weight, g | 20.51 | 21.74 | 21.43 | 20.48 | 22.33 | 20.76 |
| Day 18 | | | | | | |
| Tumor Length, mm | 8.97 | 8.58 | 10.62 | 9.13 | 10.97 | 10.6 |
| Tumor Width, mm | 8.41 | 8.21 | 9.09 | 8.09 | 8.03 | 8.83 |
| Tumor Volume, mm³ | 332.02 | 302.66 | 459.23 | 312.71 | 370.18 | 432.52 |
| Mouse Weight, g | 21.94 | 22.96 | 21.98 | 23.23 | 22.44 | 21.55 |
| Day 21 | | | | | | |
| Tumor Length, mm | 10.32 | 9.85 | 10.71 | 10.99 | 9.04 | 9.86 |
| Tumor Width, mm | 9.94 | 9.85 | 8.59 | 8.23 | 8.97 | 9.35 |
| Tumor Volume, mm³ | 533.62 | 500.13 | 413.57 | 389.56 | 380.66 | 451.11 |
| Mouse Weight, g | 21.52 | 23.5 | 22.6 | 22.44 | 22.72 | 23.54 |
| Day 25 | | | | | | |
| Tumor Length, mm | 10.94 | 11.6 | 9.44 | 10.91 | 11.63 | 11.67 |
| Tumor Width, mm | 10.71 | 8.34 | 9.23 | 9.83 | 10.03 | 9.39 |
| Tumor Volume, mm³ | 656.71 | 422.25 | 420.88 | 551.71 | 612.29 | 538.49 |
| Mouse Weight, g | 25.39 | 23.46 | 22.62 | 23.08 | 23.59 | 24.95 |
| Day 28 | | | | | | |
| Tumor Length, mm | 11.18 | 12.43 | 12.8 | 11.18 | 10.15 | 10.49 |
| Tumor Width, mm | 9.72 | 10.02 | 9.93 | 10.84 | 9.98 | 9.95 |
| Tumor Volume, mm³ | 552.78 | 653.11 | 660.52 | 687.51 | 529.06 | 543.50 |
| Mouse Weight, g | 25.77 | 25.3 | 25.77 | 25.38 | 26.19 | 24.2 |

TABLE 6-3
IgG4 (5 mg/kg) Group (Ex. 2)

| Number | 1 | 2 | 3 | 4 | 5 | 6 |
|---|---|---|---|---|---|---|
| Day 0 | | | | | | |
| Tumor Length, mm | 6.27 | 8.76 | 6.59 | 7.42 | 6.33 | 6.02 |
| Tumor Width, mm | 4.68 | 4.66 | 5.11 | 4.9 | 5.55 | 4.95 |
| Tumor Volume, mm³ | 71.87 | 99.55 | 90.05 | 93.23 | 102.04 | 77.19 |
| Mouse Weight, g | 20.36 | 20.87 | 20.94 | 20.91 | 20.47 | 20.01 |
| Day 4 | | | | | | |
| Tumor Length, mm | 6.09 | 6.4 | 7.29 | 6.97 | 6.34 | 6.38 |
| Tumor Width, mm | 5.98 | 5.82 | 5.05 | 5.85 | 5.61 | 4.83 |
| Tumor Volume, mm³ | 113.97 | 113.45 | 97.29 | 124.83 | 104.42 | 77.89 |
| Mouse Weight, g | 21.08 | 20.39 | 19.52 | 20.64 | 20.51 | 20.86 |
| Day 7 | | | | | | |
| Tumor Length, mm | 7.28 | 7.28 | 7.86 | 8.67 | 6.99 | 8.27 |
| Tumor Width, mm | 6.89 | 5.56 | 6.94 | 5.57 | 6.89 | 5.61 |
| Tumor Volume, mm³ | 180.86 | 117.78 | 198.12 | 140.77 | 173.66 | 136.21 |
| Mouse Weight, g | 20.12 | 20.89 | 20.46 | 20.95 | 20.41 | 20.14 |
| Day 11 | | | | | | |
| Tumor Length, mm | 8.11 | 8.29 | 6.98 | 8.22 | 7.53 | 8.23 |
| Tumor Width, mm | 5.81 | 7.35 | 5.72 | 8.04 | 7.15 | 7.08 |
| Tumor Volume, mm³ | 143.27 | 234.37 | 119.52 | 278.08 | 201.46 | 215.90 |
| Mouse Weight, g | 20.62 | 21.41 | 21.07 | 21.28 | 21.63 | 21.8 |
| Day 14 | | | | | | |
| Tumor Length, mm | 8.23 | 8.98 | 8.47 | 7.26 | 9.3 | 9.49 |
| Tumor Width, mm | 7.18 | 8.09 | 7.53 | 7.08 | 8.12 | 8.65 |
| Tumor Volume, mm³ | 222.04 | 307.58 | 251.33 | 190.45 | 320.90 | 371.60 |
| Mouse Weight, g | 21.45 | 21.54 | 21.59 | 20.77 | 20.99 | 20.79 |
| Day 18 | | | | | | |
| Tumor Length, mm | 8.68 | 10.39 | 9.13 | 9.05 | 9.45 | 9.95 |
| Tumor Width, mm | 8.45 | 9.93 | 8.96 | 8.53 | 8.23 | 8.35 |
| Tumor Volume, mm³ | 324.35 | 536.16 | 383.59 | 344.61 | 334.97 | 363.06 |
| Mouse Weight, g | 21.11 | 21.64 | 21.17 | 23.02 | 21.37 | 21.65 |
| Day 21 | | | | | | |
| Tumor Length, mm | 10.54 | 10.95 | 9.73 | 10.89 | 10.09 | 10.17 |
| Tumor Width, mm | 9.04 | 10.51 | 9.13 | 9.42 | 9.37 | 9.57 |
| Tumor Volume, mm³ | 450.77 | 632.99 | 424.46 | 505.72 | 463.61 | 487.44 |
| Mouse Weight, g | 22.1 | 21.89 | 21.53 | 22.19 | 22.05 | 22.31 |
| Day 25 | | | | | | |
| Tumor Length, mm | 12.37 | 11.71 | 10.99 | 10.99 | 12.36 | 12.63 |
| Tumor Width, mm | 10.58 | 11.04 | 9.93 | 9.56 | 9.94 | 10.28 |
| Tumor Volume, mm³ | 724.64 | 746.92 | 567.12 | 525.64 | 639.10 | 698.50 |
| Mouse Weight, g | 23.34 | 23.42 | 22.84 | 23.04 | 24.57 | 25.4 |
| Day 28 | | | | | | |
| Tumor Length, mm | 11.25 | 11.92 | 12.46 | 12.52 | 12.24 | 12.94 |
| Tumor Width, mm | 11.12 | 10.74 | 11.35 | 11.93 | 11.42 | 11.65 |
| Tumor Volume, mm³ | 728.02 | 719.55 | 840.02 | 932.53 | 835.40 | 919.10 |
| Mouse Weight, g | 25.62 | 23.8 | 26.56 | 23.98 | 25.66 | 24.9 |

TABLE 6-4
Cisplatin (4 mg/kg) Group (C. Ex. 2)

| Number | 1 | 2 | 3 | 4 | 5 | 6 |
|---|---|---|---|---|---|---|
| Day 0 | | | | | | |
| Tumor Length, mm | 6.73 | 6.19 | 6.27 | 6.28 | 7.76 | 7.8 |
| Tumor Width, mm | 4.15 | 5.35 | 5.12 | 4.74 | 4.83 | 5.18 |
| Tumor Volume, mm³ | 60.66 | 92.72 | 86.02 | 73.84 | 94.74 | 109.53 |
| Mouse Weight, g | 20.24 | 20.99 | 19.91 | 20.78 | 20.29 | 20.61 |
| Day 4 | | | | | | |
| Tumor Length, mm | 6.66 | 6.27 | 5.94 | 5.93 | 6.62 | 5.98 |
| Tumor Width, mm | 5.02 | 5.62 | 5.85 | 5.83 | 6.92 | 5.56 |
| Tumor Volume, mm³ | 87.83 | 103.64 | 106.38 | 105.48 | 165.90 | 96.75 |
| Mouse Weight, g | 20.91 | 20.32 | 20.42 | 20.81 | 20.93 | 19.35 |
| Day 7 | | | | | | |
| Tumor Length, mm | 6.33 | 6.99 | 6.68 | 7.3 | 7.66 | 7.34 |
| Tumor Width, mm | 5.42 | 5.52 | 6.31 | 6.16 | 6.79 | 6.19 |
| Tumor Volume, mm³ | 97.32 | 111.46 | 139.19 | 144.96 | 184.82 | 147.18 |
| Mouse Weight, g | 19.77 | 20.8 | 21.42 | 19.96 | 20.93 | 20.38 |
| Day 11 | | | | | | |
| Tumor Length, mm | 8.7 | 6.68 | 7.84 | 8.41 | 7.57 | 7.94 |
| Tumor Width, mm | 6.55 | 6.18 | 6.08 | 7.57 | 7.19 | 5.43 |
| Tumor Volume, mm³ | 195.34 | 133.52 | 151.67 | 252.21 | 204.80 | 122.52 |
| Mouse Weight, g | 21.17 | 21.76 | 20.58 | 20.08 | 21.59 | 19.94 |
| Day 14 | | | | | | |
| Tumor Length, mm | 7.16 | 8.08 | 7.68 | 8.19 | 9.8 | 9.87 |
| Tumor Width, mm | 7.15 | 7.04 | 7.16 | 8.09 | 7.64 | 7.05 |
| Tumor Volume, mm³ | 191.56 | 209.57 | 206.05 | 280.52 | 299.36 | 256.73 |
| Mouse Weight, g | 21.09 | 21.3 | 21.38 | 21.68 | 20.56 | 22.59 |
| Day 18 | | | | | | |
| Tumor Length, mm | 9.5 | 10.13 | 8.93 | 9.14 | 8.11 | 9.96 |
| Tumor Width, mm | 8.5 | 8.24 | 8.1 | 8.76 | 7.45 | 8.24 |
| Tumor Volume, mm³ | 359.20 | 359.95 | 306.62 | 367.06 | 235.57 | 353.91 |
| Mouse Weight, g | 21.17 | 21.02 | 21.25 | 21.23 | 21.03 | 21.58 |
| Day 21 | | | | | | |
| Tumor Length, mm | 9.64 | 9.79 | 9.77 | 10.69 | 9.27 | 9.57 |
| Tumor Width, mm | 9.56 | 8.08 | 8.83 | 9.91 | 8.83 | 8.93 |
| Tumor Volume, mm³ | 461.07 | 334.49 | 398.65 | 549.42 | 378.25 | 399.39 |
| Mouse Weight, g | 22.54 | 22.44 | 22.47 | 22.85 | 22.23 | 22.65 |
| Day 25 | | | | | | |
| Tumor Length, mm | 10.14 | 11.63 | 10.89 | 10.35 | 10.8 | 9.29 |
| Tumor Width, mm | 9.18 | 10.32 | 9.78 | 9.09 | 9.82 | 8.54 |
| Tumor Volume, mm³ | 447.20 | 648.21 | 545.11 | 447.56 | 545.04 | 354.58 |
| Mouse Weight, g | 25.05 | 24.06 | 22.26 | 23.7 | 25.49 | 22.66 |
| Day 28 | | | | | | |
| Tumor Length, mm | 10.94 | 11.95 | 11.36 | 9.95 | 11.27 | 11.26 |
| Tumor Width, mm | 9.08 | 10.15 | 10.19 | 8.76 | 10.29 | 10.75 |
| Tumor Volume, mm³ | 472.03 | 644.29 | 617.31 | 399.59 | 624.50 | 680.98 |
| Mouse Weight, g | 24.11 | 25.57 | 24.32 | 23.69 | 24.47 | 25.73 |

TABLE 6-5
Cisplatin (4 mg/kg) + IgG1 (1.5 mg/kg) Group (Ex. 3)

| Number | 1 | 2 | 3 | 4 | 5 | 6 |
|---|---|---|---|---|---|---|
| Day 0 | | | | | | |
| Tumor Length, mm | 6.33 | 6.5 | 6.91 | 6.49 | 6.77 | 6.01 |
| Tumor Width, mm | 4.54 | 4.66 | 5.29 | 4.46 | 5.33 | 5.96 |
| Tumor Volume, mm³ | 68.28 | 73.87 | 101.20 | 67.56 | 100.65 | 111.72 |
| Mouse Weight, g | 20.45 | 20.72 | 20.51 | 20.96 | 19.94 | 20.14 |
| Day 4 | | | | | | |
| Tumor Length, mm | 7.09 | 7.16 | 8.31 | 7.03 | 7.23 | 5.72 |
| Tumor Width, mm | 5.37 | 6.01 | 4.62 | 5.85 | 6.12 | 5.47 |
| Tumor Volume, mm³ | 107.00 | 135.34 | 92.82 | 125.91 | 141.72 | 89.57 |
| Mouse Weight, g | 20.17 | 19.95 | 19.56 | 20.53 | 20.45 | 20.24 |
| Day 7 | | | | | | |
| Tumor Length, mm | 6.23 | 6.35 | 7.2 | 7.57 | 7.08 | 7.86 |
| Tumor Width, mm | 5.66 | 6.27 | 6.65 | 5.71 | 6.38 | 6.61 |
| Tumor Volume, mm³ | 104.45 | 130.64 | 166.63 | 129.17 | 150.82 | 179.72 |
| Mouse Weight, g | 19.97 | 20.81 | 20.34 | 20.98 | 20.03 | 20.15 |
| Day 11 | | | | | | |
| Tumor Length, mm | 7.22 | 7.78 | 8.14 | 6.32 | 8.4 | 7.89 |
| Tumor Width, mm | 7.15 | 6.57 | 6.37 | 6.18 | 6.24 | 6.96 |
| Tumor Volume, mm³ | 193.16 | 175.75 | 172.85 | 126.32 | 171.17 | 200.02 |
| Mouse Weight, g | 20.63 | 21.27 | 21.79 | 21.43 | 20.27 | 20.64 |
| Day 14 | | | | | | |
| Tumor Length, mm | 9.05 | 9.23 | 7.87 | 8.23 | 8.32 | 8.07 |
| Tumor Width, mm | 7.16 | 7.32 | 7.02 | 6.55 | 6.51 | 7.38 |
| Tumor Volume, mm³ | 242.80 | 258.82 | 202.97 | 184.78 | 184.53 | 230.02 |
| Mouse Weight, g | 20.65 | 20.69 | 21.71 | 21.17 | 21.4 | 20.96 |
| Day 18 | | | | | | |
| Tumor Length, mm | 8.63 | 9.32 | 8.33 | 8.87 | 10.1 | 9.69 |
| Tumor Width, mm | 8.3 | 7.5 | 7.82 | 7.51 | 6.2 | 8.49 |
| Tumor Volume, mm³ | 311.13 | 274.36 | 266.59 | 261.81 | 203.18 | 365.53 |
| Mouse Weight, g | 21.06 | 21.11 | 22.85 | 21.93 | 22.2 | 21.22 |
| Day 21 | | | | | | |
| Tumor Length, mm | 9.8 | 9.15 | 9.36 | 9.66 | 9.26 | 9.73 |
| Tumor Width, mm | 8.71 | 9.11 | 8.2 | 8.55 | 7.96 | 8.15 |
| Tumor Volume, mm³ | 389.08 | 397.41 | 329.37 | 369.56 | 307.05 | 338.23 |
| Mouse Weight, g | 21.98 | 22.83 | 22.41 | 21.71 | 22.45 | 22.01 |
| Day 25 | | | | | | |
| Tumor Length, mm | 11.14 | 10.08 | 9.47 | 10.19 | 10.29 | 11.69 |
| Tumor Width, mm | 9.32 | 7.76 | 8.23 | 9.53 | 9.25 | 8.41 |
| Tumor Volume, mm³ | 506.40 | 317.66 | 335.68 | 484.33 | 460.76 | 432.70 |
| Mouse Weight, g | 25.07 | 24.82 | 22.21 | 23.39 | 25.95 | 21.71 |
| Day 28 | | | | | | |
| Tumor Length, mm | 10.88 | 9.83 | 11.97 | 10.6 | 9.28 | 10.38 |
| Tumor Width, mm | 9.38 | 9.46 | 9.28 | 9.21 | 8.15 | 9.17 |
| Tumor Volume, mm³ | 500.97 | 460.38 | 539.47 | 470.55 | 322.58 | 456.79 |
| Mouse Weight, g | 24.81 | 24.71 | 26.05 | 25.15 | 25.1 | 26.42 |

TABLE 6-6
Cisplatin (4 mg/kg) + IgG4 (1.5 mg/kg) Group (Ex. 4)

| Number | 1 | 2 | 3 | 4 | 5 | 6 |
|---|---|---|---|---|---|---|
| Day 0 | | | | | | |
| Tumor Length, mm | 6.63 | 6.96 | 7.24 | 6.11 | 6.6 | 5.42 |
| Tumor Width, mm | 5.09 | 5.32 | 5.49 | 4.43 | 4.92 | 5.06 |
| Tumor Volume, mm³ | 89.89 | 103.09 | 114.20 | 62.75 | 83.61 | 72.62 |
| Mouse Weight, g | 20.62 | 20.26 | 20.22 | 20.07 | 20.14 | 20.68 |
| Day 4 | | | | | | |
| Tumor Length, mm | 5.29 | 5.78 | 8.32 | 7.39 | 8.6 | 7.12 |
| Tumor Width, mm | 5.23 | 5.6 | 5.15 | 5.29 | 5.23 | 5.51 |
| Tumor Volume, mm³ | 75.72 | 94.86 | 115.48 | 108.23 | 123.11 | 113.13 |
| Mouse Weight, g | 19.65 | 20.31 | 19.82 | 20.81 | 20.05 | 20.69 |
| Day 7 | | | | | | |
| Tumor Length, mm | 6.88 | 6.56 | 7.11 | 6.97 | 7.34 | 7.17 |
| Tumor Width, mm | 6.69 | 6.41 | 5.66 | 6.48 | 5.62 | 6.5 |
| Tumor Volume, mm³ | 161.15 | 141.06 | 119.20 | 153.17 | 121.32 | 158.53 |
| Mouse Weight, g | 19.64 | 20.54 | 20.02 | 20.69 | 20.94 | 20.72 |
| Day 11 | | | | | | |
| Tumor Length, mm | 8.56 | 8.84 | 7.68 | 8.51 | 5.24 | 7.99 |
| Tumor Width, mm | 7.96 | 6.76 | 6.19 | 6.98 | 8.37 | 5.78 |
| Tumor Volume, mm³ | 283.84 | 211.41 | 154.00 | 216.98 | 192.11 | 139.69 |
| Mouse Weight, g | 21.51 | 21.02 | 20.39 | 20.93 | 20.35 | 21.08 |
| Day 14 | | | | | | |
| Tumor Length, mm | 8.05 | 8.56 | 8.06 | 8.5 | 9.97 | 8.25 |
| Tumor Width, mm | 6.81 | 7.17 | 6.54 | 6.6 | 7.83 | 7.31 |
| Tumor Volume, mm³ | 195.37 | 230.30 | 180.41 | 193.77 | 319.89 | 230.71 |
| Mouse Weight, g | 21.23 | 21.63 | 21.29 | 21.52 | 21.98 | 21.21 |
| Day 18 | | | | | | |
| Tumor Length, mm | 9.9 | 10.03 | 9.57 | 10.01 | 8.67 | 8.95 |
| Tumor Width, mm | 8.6 | 8.54 | 8.81 | 8.15 | 8.27 | 8.19 |
| Tumor Volume, mm³ | 383.19 | 382.82 | 388.72 | 347.96 | 310.32 | 314.17 |
| Mouse Weight, g | 22.6 | 21.08 | 22.87 | 22.36 | 21.35 | 22.8 |
| Day 21 | | | | | | |
| Tumor Length, mm | 10.78 | 10.68 | 8.98 | 9.96 | 9.29 | 10.64 |
| Tumor Width, mm | 8.59 | 8.96 | 8.06 | 8.07 | 8.88 | 9.94 |
| Tumor Volume, mm³ | 416.28 | 448.71 | 305.30 | 339.46 | 383.37 | 550.16 |
| Mouse Weight, g | 22.69 | 21.76 | 21.73 | 22.84 | 22.37 | 22.34 |
| Day 25 | | | | | | |
| Tumor Length, mm | 10.72 | 10.59 | 10.55 | 11.06 | 10.75 | 10.45 |
| Tumor Width, mm | 9.2 | 9.43 | 8.75 | 10.48 | 8.5 | 8.54 |
| Tumor Volume, mm³ | 474.84 | 492.83 | 422.71 | 635.71 | 406.47 | 398.85 |
| Mouse Weight, g | 24.93 | 23.07 | 25.7 | 24.69 | 24.5 | 22.57 |
| Day 28 | | | | | | |
| Tumor Length, mm | 10.24 | 11.59 | 10.69 | 10.85 | 11.56 | 10.65 |
| Tumor Width, mm | 9.64 | 10.3 | 8.13 | 9.52 | 10.19 | 9.66 |
| Tumor Volume, mm³ | 498.00 | 643.48 | 369.77 | 514.61 | 628.18 | 520.09 |
| Mouse Weight, g | 24.59 | 23.79 | 24.49 | 25.52 | 24.43 | 24.02 |

TABLE 6-7
Cisplatin (4 mg/kg) + IgG1 (5 mg/kg) Group (Ex. 5)

| Number | 1 | 2 | 3 | 4 | 5 | 6 |
|---|---|---|---|---|---|---|
| Day 0 | | | | | | |
| Tumor Length, mm | 6.03 | 5.33 | 7.28 | 6.56 | 7.82 | 7.66 |
| Tumor Width, mm | 5.52 | 5.32 | 4.95 | 4.41 | 5.55 | 4.93 |
| Tumor Volume, mm³ | 96.16 | 78.95 | 93.35 | 66.77 | 126.06 | 97.43 |
| Mouse Weight, g | 20.35 | 20.08 | 19.9 | 20.64 | 20.48 | 20.4 |
| Day 4 | | | | | | |
| Tumor Length, mm | 6.13 | 5.55 | 5.16 | 7.74 | 6.82 | 6.29 |
| Tumor Width, mm | 5.24 | 5.37 | 5.06 | 6.04 | 5.98 | 5.97 |
| Tumor Volume, mm³ | 88.08 | 83.76 | 69.14 | 147.77 | 127.63 | 117.32 |
| Mouse Weight, g | 19.87 | 20.97 | 20.47 | 20.3 | 19.79 | 20.53 |
| Day 7 | | | | | | |
| Tumor Length, mm | 7.57 | 6.88 | 8.3 | 8.1 | 6.93 | 6.34 |
| Tumor Width, mm | 5.33 | 5.54 | 6.62 | 5.45 | 5.84 | 5.04 |
| Tumor Volume, mm³ | 112.55 | 110.51 | 190.36 | 125.91 | 123.69 | 84.28 |
| Mouse Weight, g | 20.37 | 20.99 | 19.93 | 21.57 | 20 | 19.83 |
| Day 11 | | | | | | |
| Tumor Length, mm | 8.32 | 6.73 | 8.81 | 7.22 | 6.54 | 6.22 |
| Tumor Width, mm | 6.28 | 6.55 | 6.82 | 5.81 | 6.09 | 5.83 |
| Tumor Volume, mm³ | 171.72 | 151.10 | 214.45 | 127.55 | 126.94 | 110.64 |
| Mouse Weight, g | 21.5 | 20.47 | 20.11 | 20.65 | 21.77 | 20.19 |
| Day 14 | | | | | | |
| Tumor Length, mm | 8.36 | 8.38 | 9.82 | 7.47 | 8.3 | 7.86 |
| Tumor Width, mm | 8.03 | 5.34 | 5.18 | 5.54 | 7.36 | 6.76 |
| Tumor Volume, mm³ | 282.11 | 125.06 | 137.90 | 119.98 | 235.29 | 187.97 |
| Mouse Weight, g | 21.25 | 21.39 | 21.49 | 21.62 | 20.77 | 22.45 |
| Day 18 | | | | | | |
| Tumor Length, mm | 8.96 | 7.98 | 9.73 | 8.1 | 8.32 | 9.69 |
| Tumor Width, mm | 7.07 | 6.6 | 7.12 | 6.73 | 6.76 | 6.27 |
| Tumor Volume, mm³ | 234.38 | 181.92 | 258.14 | 192.00 | 198.97 | 199.36 |
| Mouse Weight, g | 21 | 21.15 | 21.19 | 21.7 | 21.35 | 21.46 |
| Day 21 | | | | | | |
| Tumor Length, mm | 9.64 | 9.94 | 8.82 | 8.67 | 9.81 | 7.34 |
| Tumor Width, mm | 6.41 | 7.34 | 7.37 | 8.21 | 7.04 | 7.1 |
| Tumor Volume, mm³ | 207.29 | 280.26 | 250.72 | 305.83 | 254.44 | 193.64 |
| Mouse Weight, g | 21.96 | 22.95 | 22.58 | 21.77 | 22.07 | 22.31 |
| Day 25 | | | | | | |
| Tumor Length, mm | 8.13 | 9.8 | 9.75 | 9.85 | 8.51 | 8.51 |
| Tumor Width, mm | 7.33 | 8.15 | 7.77 | 8.39 | 7.85 | 6.51 |
| Tumor Volume, mm³ | 228.60 | 340.66 | 308.05 | 362.86 | 274.44 | 188.74 |
| Mouse Weight, g | 23.44 | 22.64 | 24.58 | 22.38 | 24.56 | 25.09 |
| Day 28 | | | | | | |
| Tumor Length, mm | 9.64 | 10.49 | 10.66 | 9.18 | 9.17 | 9.64 |
| Tumor Width, mm | 7.35 | 8.19 | 7.76 | 6.91 | 8.3 | 7.03 |
| Tumor Volume, mm³ | 272.54 | 368.23 | 335.94 | 229.39 | 330.60 | 249.33 |
| Mouse Weight, g | 26.29 | 25.63 | 25 | 24.01 | 26.01 | 25.66 |

TABLE 6-8
Cisplatin (4 mg/kg) + IgG4 (1.5 mg/kg) Group (Ex. 6)

| Number | 1 | 2 | 3 | 4 | 5 | 6 |
|---|---|---|---|---|---|---|
| Day 0 | | | | | | |
| Tumor Length, mm | 6.73 | 6.6 | 6.66 | 5.17 | 6.85 | 6.64 |
| Tumor Width, mm | 4.95 | 5.43 | 4.92 | 5.06 | 4.95 | 5.48 |
| Tumor Volume, mm³ | 86.30 | 101.84 | 84.37 | 69.27 | 87.84 | 104.35 |
| Mouse Weight, g | 20.51 | 20.02 | 20.23 | 20.86 | 20.67 | 20.86 |
| Day 4 | | | | | | |
| Tumor Length, mm | 6.9 | 7.08 | 7.13 | 6.16 | 7.27 | 6.44 |
| Tumor Width, mm | 6.15 | 5.14 | 5.44 | 5.29 | 6.07 | 5.38 |
| Tumor Volume, mm³ | 136.58 | 97.89 | 110.42 | 90.21 | 140.18 | 97.55 |
| Mouse Weight, g | 19.47 | 20.27 | 20.42 | 20.34 | 20.51 | 20.02 |
| Day 7 | | | | | | |
| Tumor Length, mm | 6.65 | 6.86 | 7.52 | 6.52 | 7.27 | 6.92 |
| Tumor Width, mm | 6.37 | 5.84 | 6.05 | 5.98 | 7.13 | 6.49 |
| Tumor Volume, mm³ | 141.21 | 122.44 | 144.05 | 122.02 | 193.42 | 152.54 |
| Mouse Weight, g | 20.18 | 20.94 | 20.23 | 20.88 | 20.52 | 20.38 |
| Day 11 | | | | | | |
| Tumor Length, mm | 7.63 | 8.83 | 8.69 | 6.95 | 8.92 | 6.65 |
| Tumor Width, mm | 6.55 | 5.75 | 6.59 | 5.99 | 7.66 | 6.23 |
| Tumor Volume, mm³ | 171.31 | 152.78 | 197.50 | 130.50 | 273.91 | 135.08 |
| Mouse Weight, g | 20.93 | 21.25 | 21.06 | 21.58 | 20.3 | 21.03 |
| Day 14 | | | | | | |
| Tumor Length, mm | 9.14 | 8.97 | 8.65 | 8.86 | 7.12 | 8.29 |
| Tumor Width, mm | 7.3 | 7.63 | 7.15 | 7.3 | 6.29 | 7.77 |
| Tumor Volume, mm³ | 254.90 | 273.29 | 231.42 | 247.09 | 147.42 | 261.92 |
| Mouse Weight, g | 20.54 | 20.32 | 20.81 | 20.2 | 20.29 | 21.71 |
| Day 18 | | | | | | |
| Tumor Length, mm | 8.76 | 9.52 | 8.55 | 9.57 | 9.11 | 9.69 |
| Tumor Width, mm | 7.91 | 8.59 | 8.18 | 8.09 | 8.47 | 7.59 |
| Tumor Volume, mm³ | 286.84 | 367.62 | 299.40 | 327.78 | 342.03 | 292.14 |
| Mouse Weight, g | 21.08 | 21.83 | 21.15 | 21.08 | 21.6 | 21.11 |
| Day 21 | | | | | | |
| Tumor Length, mm | 10.31 | 9.64 | 9.12 | 10.86 | 10.06 | 9.22 |
| Tumor Width, mm | 9.43 | 8.47 | 8.25 | 8.98 | 8.4 | 8.24 |
| Tumor Volume, mm³ | 479.80 | 361.93 | 324.85 | 458.31 | 371.48 | 327.61 |
| Mouse Weight, g | 22.25 | 22.3 | 22.8 | 22.05 | 23.41 | 22.36 |
| Day 25 | | | | | | |
| Tumor Length, mm | 10.83 | 9.86 | 10.24 | 9.14 | 10.54 | 10.68 |
| Tumor Width, mm | 9.97 | 9.48 | 8.91 | 8.99 | 8.44 | 9.63 |
| Tumor Volume, mm³ | 563.37 | 463.74 | 425.44 | 386.58 | 392.92 | 518.33 |
| Mouse Weight, g | 24.76 | 25.14 | 22.21 | 23.42 | 24.63 | 25.78 |
| Day 28 | | | | | | |
| Tumor Length, mm | 11.28 | 10.87 | 10.35 | 11.22 | 12.93 | 10.39 |
| Tumor Width, mm | 10.13 | 9.07 | 8.53 | 8.94 | 9.76 | 9.22 |
| Tumor Volume, mm³ | 605.77 | 467.97 | 394.11 | 469.30 | 644.58 | 462.23 |
| Mouse Weight, g | 23.64 | 26.53 | 23.45 | 24.81 | 26.35 | 26.44 |

Table 6-9 shows selected raw data utilized for FIG. 4.

TABLE 6-9
Tumor weight (in grams) after 28 days

| Exam- | | | | | | | |
|---|---|---|---|---|---|---|---|
| ple | Group | 1 | 2 | 3 | 4 | 5 | 6 |
| C. | Control IgG | 0.875 | 1.007 | 0.814 | 0.895 | 0.964 | 1.127 |
| Ex. 1 | | | | | | | |
| C. | Cisplatin | 0.556 | 0.459 | 0.538 | 0.669 | 0.566 | 0.419 |
| Ex. 2 | (4 mg/kg) | | | | | | |
| Ex. 1 | IgG1 (5 mg/kg) | 0.645 | 0.585 | 0.501 | 0.618 | 0.641 | 0.555 |
| Ex. 2 | IgG4 (5 mg/kg) | 0.779 | 0.769 | 0.739 | 0.863 | 0.852 | 0.649 |
| Ex. 3 | Cisplatin + IgG1 (4 | 0.543 | 0.406 | 0.422 | 0.427 | 0.559 | 0.414 |
| | mg/kg + 1.5 mg/kg) | | | | | | |
| Ex. 4 | Cisplatin + IgG4 (4 | 0.526 | 0.546 | 0.402 | 0.474 | 0.624 | 0.501 |
| | mg/kg + 1.5 mg/kg) | | | | | | |
| Ex. 5 | Cisplatin + IgG1 (4 | 0.131 | 0.281 | 0.213 | 0.123 | 0.187 | 0.188 |
| | mg/kg + 5 mg/kg) | | | | | | |
| Ex. 6 | Cisplatin + IgG4 (4 | 0.405 | 0.585 | 0.483 | 0.413 | 0.541 | 0.581 |
| | mg/kg + 5 mg/kg) | | | | | | |

### Example 7. Amount of Anti-CD14-19G1 for Tumor Treatment

To evaluate concentrations for the anti-CD14-IgG1 antibody, PDX mice were treated with five different concentrations of the IgG1 antibody over a period of 28 days. For these experiments, drug administration on the PDX mice was performed intravenously on alternating 4 days and 3 days. Drug administration was performed on days on day 0, 4, 7, 11, 14, 18, 21, 25, and 28. Measurements of length, width, volume, and weight were performed prior to the drug administration on day 0, 4, 7, 11, 14, 18, 21, 25, and 28. The tumors were present on the skin of the mice. A ruler was used to measure the length and the width. The tumor volume was calculated by volume=½(length×width\hat{ }2). The weight of the mouse was measured by placing the mouse on a scale and weighing. Tumor weight was measured by removing the tumor from the mouse and weighing on a scale.

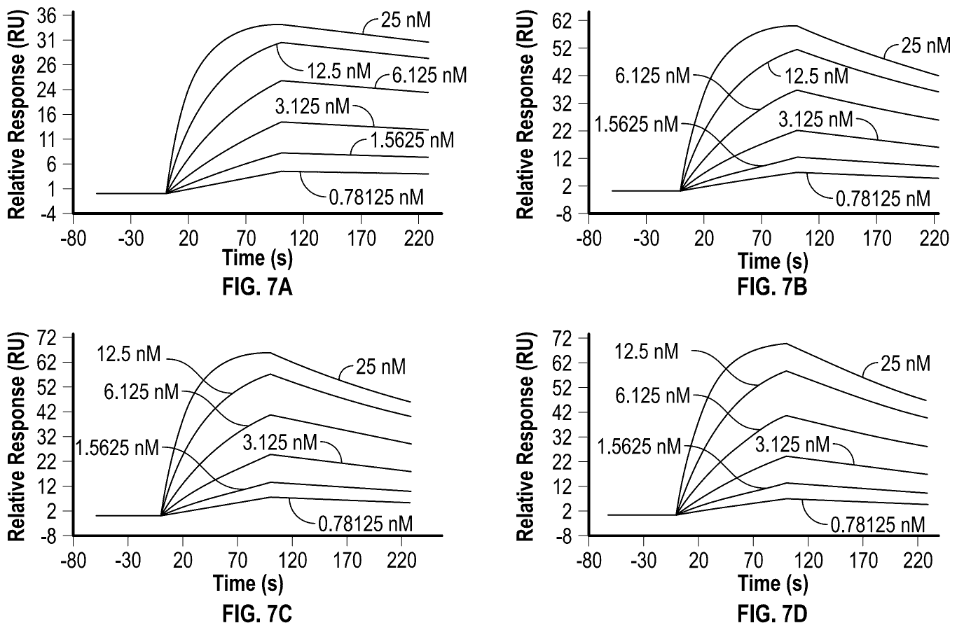

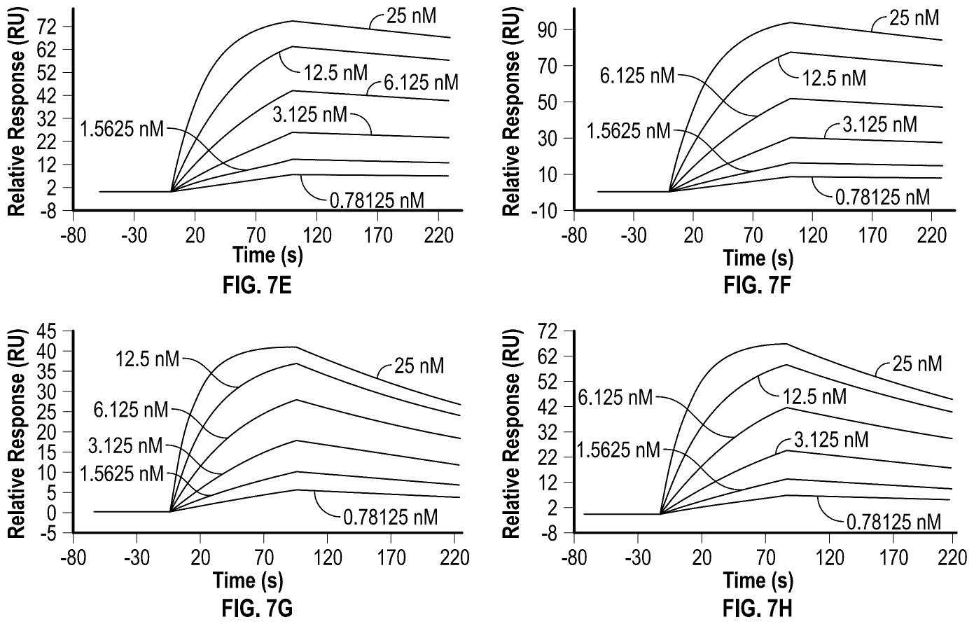

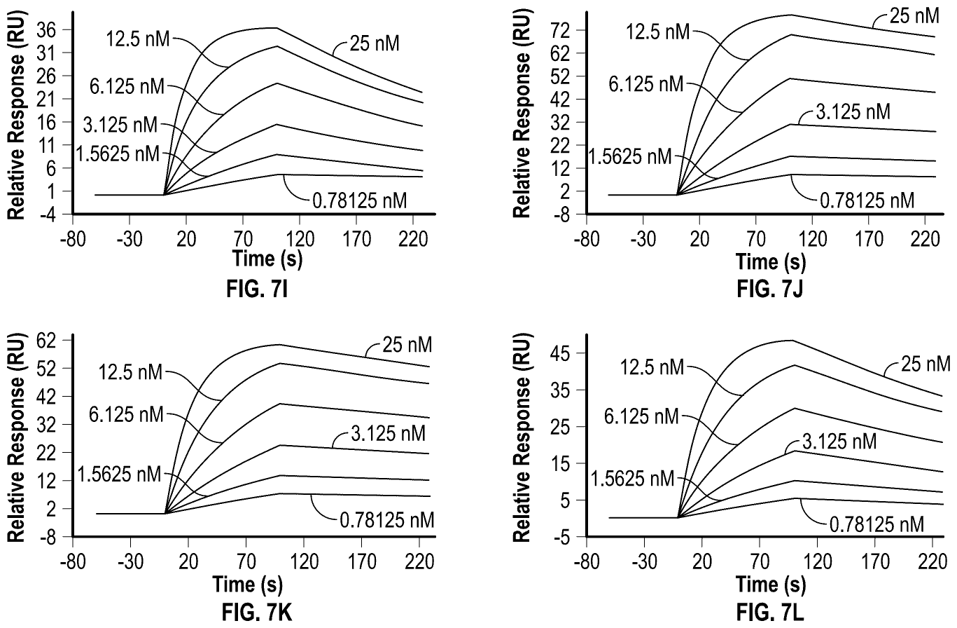

The five different concentrations of the anti-CD14-IgG1 antibody tested include 5 mg/kg, 10 mg/kg, 15 mg/kg, 25 mg/kg, and 40 mg/kg. A control regimen was utilized as a comparative example. The control is a non-specific IgG1 having two identical heavy chains and two identical light chains. The non-specific IgG1 has no binding affinity to antigen. Exemplary, but non-limiting, results of the experiments are shown in FIGS. 6A-6C. Specifically, FIG. 6A shows images of tumors collected from the PDX mice on day 28, FIG. 6B shows data for the tumor volume over the 28-day treatment, and FIG. 6C shows data for tumor weight after the 28-day treatment. Tumor weight and tumor volume were calculated as described above.

The results indicated that as the concentration of the antibody increased, there was a corresponding increase in TGI (tumor growth inhibition). Tumor inhibition level was found to significantly increase with the dosage of the anti-CD14-IgG1 antibody, ranging from 5 mg/kg to 15 mg/kg. There is an increase in tumor inhibition from 15 mg/kg to 40 mg/kg, with the tumor inhibition rate remaining the same across the three groups.

Tables 7-1 to 7-6 (below) show selected raw data used for FIG. 6B. In Tables 7-1 to 7-6, the length, width, and volume shown are the tumor dimensions, and the weight is the mouse weight. The weights of all mice under different treatments are shown. The data showed that the weights are similar and stable regardless of the dosage (ranging from 5 mg/kg to 40 mg/kg). These findings indicate that there are no safety concerns, even with a 40 mg/kg dosage of the antibody.

TABLE 7-1
Control Group

| Number | 1 | 2 | 3 | 4 | 5 |
|---|---|---|---|---|---|
| Day 0 | | | | | |
| Tumor Length, mm | 5.74 | 5.81 | 5.45 | 6.56 | 5.75 |
| Tumor Width, mm | 4.22 | 4.52 | 5.12 | 4.72 | 5.05 |
| Tumor Volume, mm³ | 53.50 | 62.12 | 74.77 | 76.48 | 76.74 |
| Mouse Weight, g | 20.15 | 20.07 | 19.07 | 20.2 | 19.22 |
| Day 4 | | | | | |
| Tumor Length, mm | 5.56 | 6.17 | 6.36 | 6.08 | 6.25 |
| Tumor Width, mm | 5.42 | 5.16 | 5.12 | 5.61 | 5.77 |
| Tumor Volume, mm³ | 85.48 | 85.97 | 87.25 | 100.14 | 108.90 |
| Mouse Weight, g | 20.94 | 20.31 | 19.99 | 20.94 | 20.25 |
| Day 7 | | | | | |
| Tumor Length, mm | 6.81 | 6.55 | 6.52 | 7.29 | 6.98 |
| Tumor Width, mm | 5.47 | 5.65 | 5.74 | 5.79 | 6.45 |
| Tumor Volume, mm³ | 106.64 | 109.43 | 112.42 | 127.90 | 151.97 |
| Mouse Weight, g | 20.55 | 20.64 | 20.76 | 20.94 | 20.77 |
| Day 11 | | | | | |
| Tumor Length, mm | 7.14 | 8.47 | 7.52 | 7.15 | 7.2 |
| Tumor Width, mm | 5.72 | 5.44 | 6.16 | 6.47 | 6.56 |
| Tumor Volume, mm³ | 122.26 | 131.18 | 149.33 | 156.64 | 162.15 |
| Mouse Weight, g | 20.18 | 20.85 | 21.34 | 20.22 | 20.89 |
| Day 14 | | | | | |
| Tumor Length, mm | 7.42 | 7.55 | 7.96 | 8.69 | 7.62 |
| Tumor Width, mm | 6.16 | 6.29 | 6.69 | 6.42 | 7.42 |
| Tumor Volume, mm³ | 147.35 | 156.32 | 186.44 | 187.44 | 219.55 |
| Mouse Weight, g | 20.94 | 20.42 | 20.78 | 21.04 | 21.72 |
| Day 18 | | | | | |
| Tumor Length, mm | 8.67 | 8.07 | 9.47 | 8.92 | 8.97 |
| Tumor Width, mm | 6.07 | 7.05 | 7.02 | 7.34 | 8.56 |
| Tumor Volume, mm³ | 167.18 | 209.91 | 244.23 | 251.50 | 343.97 |
| Mouse Weight, g | 21.63 | 20.25 | 21.37 | 20.89 | 21.69 |
| Day 21 | | | | | |
| Tumor Length, mm | 8.56 | 8.89 | 9.58 | 9.59 | 9.93 |
| Tumor Width, mm | 8.03 | 8.05 | 8.06 | 8.67 | 9.75 |
| Tumor Volume, mm³ | 288.86 | 301.49 | 325.70 | 377.26 | 494.01 |
| Mouse Weight, g | 21.26 | 21.23 | 21.83 | 22.24 | 22.1 |
| Day 25 | | | | | |
| Tumor Length, mm | 9.83 | 10.82 | 10.88 | 11.52 | 12.28 |
| Tumor Width, mm | 9.37 | 9.98 | 10.31 | 10.46 | 11.27 |
| Tumor Volume, mm³ | 451.66 | 563.98 | 605.24 | 659.62 | 816.25 |
| Mouse Weight, g | 22.27 | 21.83 | 21.65 | 22.28 | 22.71 |
| Day 28 | | | | | |
| Tumor Length, mm | 13.93 | 13.14 | 12.65 | 13.62 | 14.66 |
| Tumor Width, mm | 10.19 | 10.89 | 11.74 | 11.33 | 12.5 |
| Tumor Volume, mm³ | 756.97 | 815.51 | 912.44 | 914.99 | 1198.76 |
| Mouse Weight, g | 21.52 | 23.45 | 22.66 | 23.98 | 24.01 |

TABLE 7-2
IgG1 (5 mg/kg) Group

| Number | 1 | 2 | 3 | 4 | 5 |
|---|---|---|---|---|---|
| Day 0 | 5.77 | 6.07 | 6.17 | 5.74 | 6.27 |
| Tumor Length, mm | 4.41 | 4.54 | 4.71 | 5.12 | 4.93 |
| Tumor Width, mm | 58.73 | 65.48 | 71.63 | 78.75 | 79.75 |
| Tumor Volume, mm³ | 20.01 | 19.41 | 19.14 | 19.44 | 19.33 |
| Mouse Weight, g | | | | | |
| Day 4 | | | | | |
| Tumor Length, mm | 5.58 | 6.11 | 6.53 | 5.89 | 6.64 |
| Tumor Width, mm | 5.22 | 5.24 | 5.19 | 5.53 | 5.51 |
| Tumor Volume, mm³ | 79.57 | 87.80 | 92.05 | 94.26 | 105.50 |
| Mouse Weight, g | 20.33 | 20.63 | 19.58 | 19.86 | 20.45 |
| Day 7 | | | | | |
| Tumor Length, mm | 7.84 | 6.11 | 6.37 | 6.61 | 6.56 |
| Tumor Width, mm | 5.03 | 5.73 | 5.73 | 5.92 | 6.22 |
| Tumor Volume, mm³ | 103.81 | 104.99 | 109.45 | 121.23 | 132.82 |
| Mouse Weight, g | 20.95 | 20.07 | 20.36 | 20.83 | 20.56 |
| Day 11 | | | | | |
| Tumor Length, mm | 6.48 | 6.63 | 6.74 | 6.44 | 7.52 |
| Tumor Width, mm | 5.22 | 5.45 | 6.04 | 6.23 | 6.35 |
| Tumor Volume, mm³ | 92.40 | 103.06 | 128.68 | 130.81 | 158.69 |
| Mouse Weight, g | 20.85 | 20.72 | 20.22 | 21.21 | 20.94 |
| Day 14 | | | | | |
| Tumor Length, mm | 7.14 | 6.88 | 7.56 | 7.02 | 8.46 |
| Tumor Width, mm | 5.86 | 6.13 | 6.56 | 6.84 | 6.46 |
| Tumor Volume, mm³ | 128.31 | 135.30 | 170.26 | 171.88 | 184.76 |
| Mouse Weight, g | 20.75 | 21.06 | 20.89 | 21.69 | 20.67 |
| Day 18 | | | | | |
| Tumor Length, mm | 7.91 | 7.24 | 8.45 | 8.16 | 8.08 |
| Tumor Width, mm | 6.04 | 6.69 | 6.47 | 7.15 | 7.4 |
| Tumor Volume, mm³ | 151.02 | 169.58 | 185.12 | 218.31 | 231.55 |
| Mouse Weight, g | 21.11 | 21.36 | 20.44 | 20.69 | 21.33 |
| Day 21 | | | | | |
| Tumor Length, mm | 8.92 | 8.5 | 8.53 | 8.19 | 8.49 |
| Tumor Width, mm | 6.41 | 7.26 | 7.65 | 7.96 | 8.07 |
| Tumor Volume, mm³ | 191.80 | 234.46 | 261.25 | 271.57 | 289.36 |
| Mouse Weight, g | 20.92 | 21.85 | 21.42 | 21.87 | 21.66 |
| Day 25 | | | | | |
| Tumor Length, mm | 8.73 | 9.67 | 9.91 | 9.97 | 9.69 |
| Tumor Width, mm | 7.17 | 8.13 | 8.63 | 9.45 | 9.61 |
| Tumor Volume, mm³ | 234.87 | 334.49 | 386.25 | 465.95 | 468.33 |
| Mouse Weight, g | 22.45 | 21.06 | 21.98 | 22.14 | 22.16 |
| Day 28 | | | | | |
| Tumor Length, mm | 9.76 | 9.79 | 10.39 | 11.02 | 10.87 |
| Tumor Width, mm | 8.08 | 9.47 | 10.11 | 10.23 | 10.59 |
| Tumor Volume, mm³ | 333.47 | 459.47 | 555.77 | 603.55 | 637.97 |
| Mouse Weight, g | 22.42 | 23.8 | 22.04 | 21.81 | 21.72 |

TABLE 7-3
IgG1 (10 mg/kg) Group

| Number | 1 | 2 | 3 | 4 | 5 |
|---|---|---|---|---|---|
| Day 0 | | | | | |
| Tumor Length, mm | 5.76 | 6.11 | 5.85 | 5.83 | 6.02 |
| Tumor Width, mm | 4.45 | 4.63 | 5.02 | 5.09 | 5.01 |
| Tumor Volume, mm³ | 59.69 | 68.55 | 77.15 | 79.05 | 79.08 |
| Mouse Weight, g | 19.83 | 20.73 | 20.13 | 20.77 | 19.27 |
| Day 4 | | | | | |
| Tumor Length, mm | 5.95 | 6.55 | 5.55 | 5.64 | 6.22 |
| Tumor Width, mm | 5.12 | 4.92 | 5.42 | 5.49 | 5.77 |
| Tumor Volume, mm³ | 81.63 | 82.98 | 85.32 | 88.96 | 108.37 |
| Mouse Weight, g | 19.89 | 20.39 | 19.36 | 20.74 | 20.98 |
| Day 7 | | | | | |
| Tumor Length, mm | 6.53 | 6.12 | 7.25 | 6.45 | 6.83 |
| Tumor Width, mm | 5.75 | 5.95 | 5.66 | 6.02 | 6.11 |
| Tumor Volume, mm³ | 112.99 | 113.39 | 121.55 | 122.33 | 133.44 |
| Mouse Weight, g | 20.81 | 19.35 | 19.89 | 20.23 | 21.08 |
| Day 11 | | | | | |
| Tumor Length, mm | 6.43 | 6.69 | 6.91 | 7.13 | 7.12 |
| Tumor Width, mm | 5.47 | 5.48 | 5.58 | 6.21 | 6.76 |
| Tumor Volume, mm³ | 100.68 | 105.14 | 112.60 | 143.90 | 170.28 |
| Mouse Weight, g | 20.77 | 20.5 | 20.97 | 21.03 | 20.94 |
| Day 14 | | | | | |
| Tumor Length, mm | 6.88 | 6.37 | 7.11 | 7.11 | 7.2 |
| Tumor Width, mm | 5.52 | 5.77 | 6.02 | 6.42 | 6.63 |
| Tumor Volume, mm³ | 109.71 | 110.99 | 134.85 | 153.36 | 165.63 |
| Mouse Weight, g | 20.15 | 20.69 | 21.19 | 21.53 | 20.71 |
| Day 18 | | | | | |
| Tumor Length, mm | 6.81 | 7.05 | 7.23 | 7.74 | 7.84 |
| Tumor Width, mm | 6.09 | 6.2 | 6.58 | 6.89 | 7.06 |
| Tumor Volume, mm³ | 132.18 | 141.82 | 163.82 | 192.29 | 204.50 |
| Mouse Weight, g | 21.27 | 20.66 | 21.04 | 20.85 | 21.37 |
| Day 21 | | | | | |
| Tumor Length, mm | 8.72 | 8.62 | 8.24 | 8.55 | 8.36 |
| Tumor Width, mm | 6.92 | 6.55 | 7.17 | 7.07 | 7.83 |
| Tumor Volume, mm³ | 218.53 | 193.54 | 221.69 | 223.66 | 268.23 |
| Mouse Weight, g | 21.25 | 21.16 | 20.97 | 21.13 | 21.28 |
| Day 25 | | | | | |
| Tumor Length, mm | 8.35 | 9.09 | 9.11 | 9.54 | 9.39 |
| Tumor Width, mm | 7.22 | 7.71 | 7.94 | 8.12 | 8.28 |
| Tumor Volume, mm³ | 227.79 | 282.78 | 300.56 | 329.18 | 336.90 |
| Mouse Weight, g | 22.27 | 22.35 | 21.64 | 20.37 | 22.93 |
| Day 28 | | | | | |
| Tumor Length, mm | 8.61 | 9.13 | 8.88 | 9.92 | 9.39 |
| Tumor Width, mm | 7.16 | 8.1 | 8.54 | 8.8 | 9.24 |
| Tumor Volume, mm³ | 231.00 | 313.49 | 338.93 | 402.03 | 419.55 |
| Mouse Weight, g | 22.71 | 23.8 | 21.44 | 22.24 | 21.16 |

TABLE 7-4
IgG1 (15 mg/kg) Group

| Number | 1 | 2 | 3 | 4 | 5 |
|---|---|---|---|---|---|
| Day 0 | | | | | |
| Tumor Length, mm | 5.23 | 5.95 | 5.17 | 6.14 | 5.87 |
| Tumor Width, mm | 4.84 | 4.54 | 5.04 | 4.95 | 5.22 |
| Tumor Volume, mm³ | 64.12 | 64.18 | 68.73 | 78.73 | 83.71 |
| Mouse Weight, g | 20.37 | 19.02 | 20.34 | 19.62 | 19.24 |
| Day 4 | | | | | |
| Tumor Length, mm | 5.87 | 5.58 | 5.95 | 6.41 | 6.55 |
| Tumor Width, mm | 4.98 | 5.16 | 5.13 | 5.11 | 5.14 |
| Tumor Volume, mm³ | 76.19 | 77.75 | 81.95 | 87.59 | 90.56 |
| Mouse Weight, g | 20.83 | 19.54 | 20.87 | 20.44 | 20.68 |
| Day 7 | | | | | |
| Tumor Length, mm | 6.46 | 6.21 | 6.99 | 6.95 | 7.11 |
| Tumor Width, mm | 5.42 | 5.91 | 5.69 | 5.92 | 6.41 |
| Tumor Volume, mm³ | 99.31 | 113.51 | 118.44 | 127.47 | 152.88 |
| Mouse Weight, g | 19.65 | 20.62 | 20.38 | 20.56 | 20.71 |
| Day 11 | | | | | |
| Tumor Length, mm | 6.09 | 6.86 | 6.98 | 6.96 | 7.01 |
| Tumor Width, mm | 5.61 | 5.58 | 5.66 | 6.13 | 6.24 |
| Tumor Volume, mm³ | 100.30 | 111.78 | 117.02 | 136.87 | 142.85 |
| Mouse Weight, g | 20.35 | 20.89 | 21.32 | 21.08 | 20.47 |
| Day 14 | | | | | |
| Tumor Length, mm | 6.39 | 6.51 | 6.29 | 7.66 | 7.45 |
| Tumor Width, mm | 5.25 | 5.76 | 6.26 | 6.32 | 6.66 |
| Tumor Volume, mm³ | 92.17 | 113.03 | 129.00 | 160.12 | 172.94 |
| Mouse Weight, g | 20.47 | 21.16 | 21.65 | 20.87 | 21.13 |
| Day 18 | | | | | |
| Tumor Length, mm | 6.23 | 7.25 | 6.63 | 7.04 | 7.02 |
| Tumor Width, mm | 6.21 | 5.96 | 6.62 | 6.69 | 6.75 |
| Tumor Volume, mm³ | 125.73 | 134.77 | 152.06 | 164.89 | 167.39 |
| Mouse Weight, g | 20.73 | 21.69 | 20.31 | 20.9 | 21.29 |
| Day 21 | | | | | |
| Tumor Length, mm | 7.34 | 8.07 | 8.37 | 8.27 | 8.98 |
| Tumor Width, mm | 6.12 | 6.75 | 7.31 | 7.46 | 7.33 |
| Tumor Volume, mm³ | 143.87 | 192.42 | 234.07 | 240.86 | 252.50 |
| Mouse Weight, g | 20.6 | 21.12 | 21.33 | 20.4 | 21.98 |
| Day 25 | | | | | |
| Tumor Length, mm | 9.07 | 8.03 | 8.84 | 8.33 | 9.1 |
| Tumor Width, mm | 7.02 | 7.55 | 7.23 | 7.51 | 8.14 |
| Tumor Volume, mm³ | 233.92 | 239.55 | 241.83 | 245.87 | 315.55 |
| Mouse Weight, g | 22.28 | 21.01 | 22.42 | 20.52 | 22.73 |
| Day 28 | | | | | |
| Tumor Length, mm | 8.58 | 8.23 | 9.8 | 9.32 | 8.7 |
| Tumor Width, mm | 7.38 | 7.75 | 7.94 | 8.18 | 8.5 |
| Tumor Volume, mm³ | 244.56 | 258.69 | 323.33 | 326.36 | 328.95 |
| Mouse Weight, g | 22.57 | 21.49 | 23.11 | 21.72 | 23.44 |

TABLE 7-5
IgG1 (25 mg/kg) Group

| Number | 1 | 2 | 3 | 4 | 5 |
|---|---|---|---|---|---|
| Day 0 | | | | | |
| Tumor Length, mm | 5.42 | 5.13 | 6.41 | 5.72 | 6.07 |
| Tumor Width, mm | 4.76 | 5.04 | 4.75 | 5.03 | 5.11 |
| Tumor Volume, mm³ | 64.27 | 68.20 | 75.69 | 75.74 | 82.95 |
| Mouse Weight, g | 19.63 | 20.3 | 19.71 | 20.07 | 20.02 |
| Day 4 | | | | | |
| Tumor Length, mm | 6.23 | 6.18 | 5.59 | 6.01 | 5.99 |
| Tumor Width, mm | 4.75 | 5.04 | 5.44 | 5.32 | 5.35 |
| Tumor Volume, mm³ | 73.56 | 82.15 | 86.57 | 89.02 | 89.72 |
| Mouse Weight, g | 20.75 | 19.2 | 20.99 | 20.16 | 20.55 |
| Day 7 | | | | | |
| Tumor Length, mm | 6.41 | 7.46 | 6.71 | 6.92 | 6.57 |
| Tumor Width, mm | 5.41 | 5.28 | 5.68 | 5.78 | 6.24 |
| Tumor Volume, mm³ | 98.18 | 108.84 | 113.29 | 120.99 | 133.88 |
| Mouse Weight, g | 20.86 | 20.2 | 20.76 | 21.38 | 20.06 |
| Day 11 | | | | | |
| Tumor Length, mm | 5.98 | 5.91 | 6.63 | 6.6 | 6.66 |
| Tumor Width, mm | 5.59 | 5.73 | 5.66 | 5.91 | 6.57 |
| Tumor Volume, mm³ | 97.79 | 101.55 | 111.15 | 120.64 | 150.45 |
| Mouse Weight, g | 20.11 | 20.4 | 19.5 | 20.84 | 21.5 |
| Day 14 | | | | | |
| Tumor Length, mm | 6.35 | 6.38 | 6.99 | 7.13 | 6.22 |
| Tumor Width, mm | 5.39 | 5.58 | 6.08 | 6.02 | 6.54 |
| Tumor Volume, mm³ | 96.54 | 103.96 | 135.23 | 135.23 | 139.23 |
| Mouse Weight, g | 20.13 | 21.05 | 20.39 | 21.39 | 21.62 |
| Day 18 | | | | | |
| Tumor Length, mm | 7.53 | 6.38 | 6.61 | 6.49 | 7.39 |
| Tumor Width, mm | 5.24 | 5.83 | 5.96 | 6.41 | 6.78 |
| Tumor Volume, mm³ | 108.20 | 113.48 | 122.88 | 139.55 | 177.78 |
| Mouse Weight, g | 21.14 | 21.03 | 20.46 | 21.57 | 21.77 |
| Day 21 | | | | | |
| Tumor Length, mm | 8.55 | 8.21 | 7.14 | 7.86 | 8.04 |
| Tumor Width, mm | 6.04 | 6.19 | 6.92 | 6.95 | 7.16 |
| Tumor Volume, mm³ | 163.24 | 164.63 | 178.93 | 198.69 | 215.71 |
| Mouse Weight, g | 21.3 | 22.05 | 21.54 | 20.62 | 21.57 |
| Day 25 | | | | | |
| Tumor Length, mm | 7.56 | 7.69 | 7.92 | 8.39 | 8.82 |
| Tumor Width, mm | 6.92 | 7.01 | 7.12 | 7.07 | 7.91 |
| Tumor Volume, mm³ | 189.46 | 197.76 | 210.12 | 219.47 | 288.80 |
| Mouse Weight, g | 22.17 | 21.73 | 22.39 | 21.5 | 20.56 |
| Day 28 | | | | | |
| Tumor Length, mm | 8.86 | 8.91 | 8.29 | 8.79 | 9.42 |
| Tumor Width, mm | 7.03 | 7.43 | 7.78 | 7.69 | 8.01 |
| Tumor Volume, mm³ | 229.15 | 257.41 | 262.60 | 272.03 | 316.30 |
| Mouse Weight, g | 22.62 | 23.62 | 22.69 | 21.64 | 23.4 |

TABLE 7-6
IgG1 (40 mg/kg) Group

| Number | 1 | 2 | 3 | 4 | 5 |
|---|---|---|---|---|---|
| Day 0 | | | | | |
| Tumor Length, mm | 5.12 | 6.69 | 6.92 | 5.44 | 5.75 |
| Tumor Width, mm | 5.04 | 4.54 | 4.53 | 5.12 | 5.21 |
| Tumor Volume, mm³ | 68.06 | 72.16 | 74.32 | 74.63 | 81.68 |
| Mouse Weight, g | 20.64 | 20.79 | 20.06 | 20.43 | 19.66 |
| Day 4 | | | | | |
| Tumor Length, mm | 5.42 | 6.1 | 6.72 | 6.42 | 6.01 |
| Tumor Width, mm | 5.35 | 5.16 | 5.13 | 5.29 | 5.69 |
| Tumor Volume, mm³ | 81.19 | 85.00 | 92.55 | 94.02 | 101.83 |
| Mouse Weight, g | 20.37 | 20.71 | 19.79 | 20.56 | 20.73 |
| Day 7 | | | | | |
| Tumor Length, mm | 6.02 | 6.26 | 6.16 | 6.82 | 7.03 |
| Tumor Width, mm | 5.16 | 5.31 | 5.45 | 5.66 | 6.18 |
| Tumor Volume, mm³ | 83.88 | 92.37 | 95.75 | 114.34 | 140.51 |
| Mouse Weight, g | 20.29 | 20.01 | 20.53 | 21.04 | 20.66 |
| Day 11 | | | | | |
| Tumor Length, mm | 6.42 | 6.47 | 6.35 | 6.47 | 7.66 |
| Tumor Width, mm | 5.08 | 5.42 | 5.59 | 5.61 | 6.61 |
| Tumor Volume, mm³ | 86.70 | 99.47 | 103.84 | 106.56 | 175.15 |
| Mouse Weight, g | 20.49 | 21.74 | 20.21 | 21.3 | 20.92 |
| Day 14 | | | | | |
| Tumor Length, mm | 6.17 | 6.63 | 6.32 | 6.26 | 7.23 |
| Tumor Width, mm | 5.28 | 5.26 | 6.11 | 6.17 | 6.39 |
| Tumor Volume, mm³ | 90.02 | 96.00 | 123.47 | 124.72 | 154.50 |
| Mouse Weight, g | 21.19 | 20.75 | 20.83 | 20.91 | 20.61 |
| Day 18 | | | | | |
| Tumor Length, mm | 6.75 | 6.14 | 7.02 | 6.67 | 7.56 |
| Tumor Width, mm | 5.49 | 5.98 | 5.82 | 6.31 | 6.34 |
| Tumor Volume, mm³ | 106.47 | 114.91 | 124.44 | 138.98 | 159.03 |
| Mouse Weight, g | 21.32 | 21.28 | 21.36 | 20.36 | 21.14 |
| Day 21 | | | | | |
| Tumor Length, mm | 6.21 | 7.34 | 7.81 | 7.37 | 8.88 |
| Tumor Width, mm | 6.18 | 5.7 | 6.37 | 6.65 | 6.61 |
| Tumor Volume, mm³ | 124.12 | 124.80 | 165.85 | 170.56 | 203.05 |
| Mouse Weight, g | 21.75 | 20.47 | 21.5 | 21.64 | 21.77 |
| Day 25 | | | | | |
| Tumor Length, mm | 6.85 | 7.91 | 8.84 | 8.46 | 8.15 |
| Tumor Width, mm | 6.63 | 6.41 | 6.11 | 7.54 | 8.07 |
| Tumor Volume, mm³ | 157.58 | 170.09 | 172.71 | 251.70 | 277.77 |
| Mouse Weight, g | 22.33 | 21.23 | 22.07 | 21.9 | 22.06 |
| Day 28 | | | | | |
| Tumor Length, mm | 8.81 | 8.58 | 8.17 | 8.17 | 8.11 |
| Tumor Width, mm | 7.43 | 7.79 | 8.08 | 8.11 | 6.87 |
| Tumor Volume, mm³ | 254.53 | 272.48 | 279.14 | 281.22 | 200.31 |
| Mouse Weight, g | 23.59 | 23.7 | 22.4 | 21.39 | 22.91 |

Table 7-7 shows selected raw data utilized for FIG. 6C.

TABLE 7-7
Tumor weight (in grams) after 28 days

| Group | 1 | 2 | 3 | 4 | 5 |
|---|---|---|---|---|---|
| Control | 0.799 | 0.756 | 0.815 | 0.846 | 0.913 |
| IgG1 5 mg/kg | 0.486 | 0.535 | 0.567 | 0.502 | 0.625 |
| IgG1 10 mg/kg | 0.341 | 0.302 | 0.267 | 0.285 | 0.386 |
| IgG1 15 mg/kg | 0.215 | 0.202 | 0.246 | 0.253 | 0.291 |
| IgG1 25 mg/kg | 0.158 | 0.184 | 0.191 | 0.204 | 0.211 |
| IgG1 40 mg/kg | 0.128 | 0.195 | 0.199 | 0.205 | 0.206 |

### Example 8. Measurement of Binding Affinity of the Humanized Antibodies to Recombinant Human CD14

The binding affinity of example humanized antibodies to recombinant human CD14 were measured using ELISA. Antibody affinity is a measure of the strength of the binding interaction between an antigenic determinant (epitope) and the binding site (paratope) on the antibody. Antibody affinity can directly influence the efficacy and specificity of an antibody in both diagnostic and therapeutic applications.

High-affinity antibodies can ensure specific and robust binding to their target antigen, even in the presence of competing molecules, leading to more precise and reliable outcomes. In therapeutic contexts, antibodies with high affinity can engage their target more effectively, enhancing the therapeutic impact while potentially reducing off-target effects. Therefore, accurate measurement of antibody affinity can help ensure the functionality and effectiveness of the final product.

### 8.1. Experimental Materials

Experimental reagents and consumables included Protein A chip (Cytiva, 29127556). Main instruments and equipment included Biacore 8K (Cytiva), shaker (Taicang Huamei), ELISA system (Molecular Devices), centrifuge (Baiyang), ultrapure water system (Millipore), and thermostatic incubator (BLUE PARD).

The antigen used for the binding affinity measurements is B294801. This antigen is a recombinant human CD14 antigen that is histidine tagged and has a molecular weight of 36 kDa. B294801 was purchased from Shanghai BaiYing Biotechnology Co., Ltd (name: Recombinant Human CD14/His Protein; Batch number 20200518A22). D6DD3D001 is a mouse antibody, and D6DD3D002-D6DD3D0016 are humanized antibodies. The antibodies are recombinant, human (hFc Tag). Selected parameters of the experimental samples are shown in Table 8-1, with MW referring to molecular weight in kiloDaltons (kDa).

TABLE 8-1

| | Conc., | MW, | | |
|---|---|---|---|---|
| Name | mg/mL | kDa | Tag | Sample type |
| D6DD3D001 | 1.2 | 146 | hFc | Original IgG1 antibody |
| D6DD3D002 | 0.33 | 144 | hFc | Antibody |
| D6DD3D003 | 0.47 | 144 | hFc | Antibody |
| D6DD3D004 | 0.61 | 144 | hFc | Antibody |
| D6DD3D005 | 0.44 | 146 | hFc | Antibody |
| D6DD3D006 | 0.5 | 146 | hFc | Antibody |
| D6DD3D007 | 0.49 | 144 | hFc | Antibody |
| D6DD3D008 | 0.6 | 144 | hFc | Antibody |
| D6DD3D009 | 0.54 | 144 | hFc | Antibody |
| D6DD3D010 | 0.44 | 146 | hFc | Antibody |
| D6DD3D011 | 0.53 | 146 | hFc | Antibody |
| D6DD3D012 | 1.2 | 144 | hFc | Antibody |
| D6DD3D013 | 1.53 | 144 | hFc | Antibody |
| D6DD3D014 | 1.25 | 144 | hFc | Antibody |
| D6DD3D015 | 1.26 | 146 | hFc | Antibody |
| D6DD3D016 | 1.46 | 146 | hFc | Antibody |
| B294801 | 17.24 | 36 | His | Antigen |

### 8.2. Experimental Method

The Biacore 8K platform was used to test the kinetic and affinity properties of the antibody. The operation was carried out according to the system instructions and indications. The experimental parameters used are shown in Table 8-2.

TABLE 8-2
Detection parameters

| Parameter | Selected details |
|---|---|
| Capture ligand | 1 μg/mL antibody in Running Buffer, |
| | 10 μL/min |
| Running buffer | HBS-EP + Buffer (Cytiva) |
| Association and | 30 μL/min, Association 100 s, |
| dissociation flow rate | Dissociation 200 s |
| Regeneration buffer | 10 mM pH 1.5 Glycine-HCl (Gly-HCl) |
| Regeneration flow rate | 30 μL/min, 30 s |

### 8.3. Experimental Results

The equilibrium dissociation constant (KD) reflects the affinity of the antibody to the target, with a smaller value indicating a stronger affinity. KD (in units of molarity) is determined by the ratio of the dissociation rate constant (k_{off}) and the association rate constant (kₒₙ), that is, KD=K_{off}/kₒₙ.

15 humanized antibodies (numbered D6DD3D002-D6DD3D0016) and 1 mouse antibody (number D6DD3D001) were tested. For the experiments, the analyte solution comprises the B294801 antigen with an analyte concentration of 0.78125 nM, 1.5625 nM, 3.125 nM, 6.25 nM, 12.5 nM, and 25 nM. The capture concentration was 1 μg/mL. Selected results are shown in Table 8-3. In Table 8-3, the change factor is the ratio of the equilibrium dissociation constant KD of the humanized to mouse antibody, and reflects the change in affinity after humanization. A change factor equal to 1 indicates that the affinity of the humanized antibody is on par with the mouse antibody. A change factor greater than 1 indicates that the equilibrium dissociation constant KD of the humanized antibody has increased, and accordingly, the affinity has decreased. A change factor less than 1 indicates that the affinity of the humanized antibody is higher than that of the mouse antibody.

TABLE 8-3
Affinity detection results at full concentration

| | 1:1 binding | | | Change | FIG. Ref. |
|---|---|---|---|---|---|
| Ligand | kₒₙ, 1/Ms | k_{off}, 1/s | KD, M | Factor | No. |
| D6DD3D001 | 1.77E+06 | 8.58E−04 | 4.85E−10 | 1.00 | 7A |
| D6DD3D002 | 1.69E+06 | 2.92E−03 | 1.73E−09 | 3.57 | 7B |
| D6DD3D003 | 1.82E+06 | 2.95E−03 | 1.62E−09 | 3.34 | 7C |
| D6DD3D004 | 1.64E+06 | 3.26E−03 | 1.99E−09 | 4.10 | 7D |
| D6DD3D005 | 1.51E+06 | 7.98E−04 | 5.29E−10 | 1.09 | 7E |
| D6DD3D006 | 1.43E+06 | 8.62E−04 | 6.02E−10 | 1.24 | 7F |
| D6DD3D007 | 2.11E+06 | 3.42E−03 | 1.62E−09 | 3.34 | 7G |
| D6DD3D008 | 2.09E+06 | 3.29E−03 | 1.57E−09 | 3.24 | 7H |
| D6DD3D009 | 2.01E+06 | 3.79E−03 | 1.89E−09 | 3.90 | 7I |
| D6DD3D010 | 1.90E+06 | 1.05E−03 | 5.55E−10 | 1.14 | 7J |
| D6DD3D011 | 1.71E+06 | 1.09E−03 | 6.39E−10 | 1.32 | 7K |
| D6DD3D012 | 1.64E+06 | 2.93E−03 | 1.79E−09 | 3.69 | 7L |
| D6DD3D013 | 1.76E+06 | 2.82E−03 | 1.61E−09 | 3.32 | 7M |
| D6DD3D014 | 1.77E+06 | 3.18E−03 | 1.80E−09 | 3.71 | 7N |
| D6DD3D015 | 1.59E+06 | 8.43E−04 | 5.32E−10 | 1.10 | 7O |
| D6DD3D016 | 1.35E+06 | 9.04E−04 | 6.72E−10 | 1.39 | 7P |

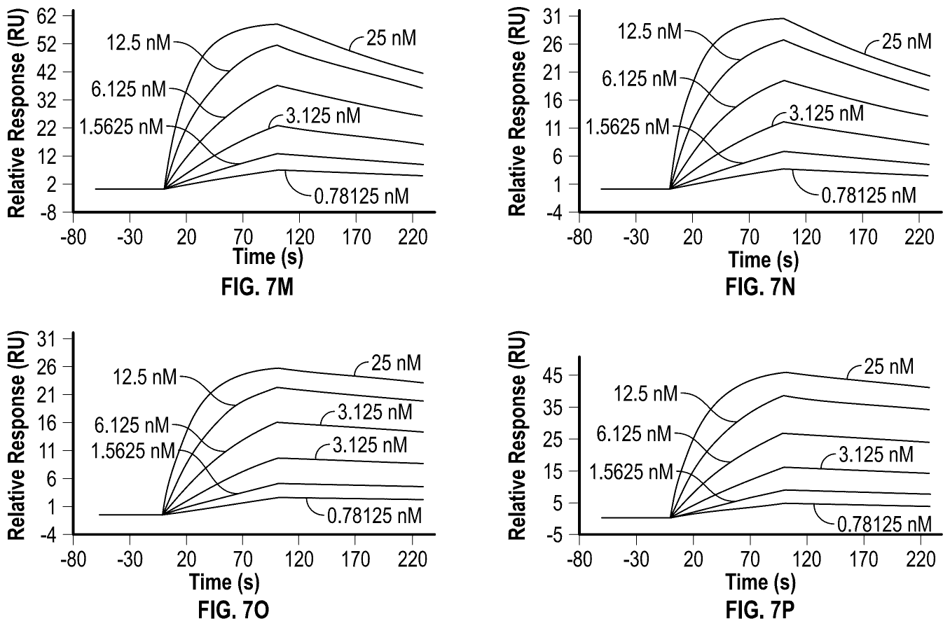

FIGS. 7A-7P show exemplary dynamic binding curves as a function of residence time with the antigen fully saturated (1:1 binding). Figure reference numbers are shown in Table 8-3. All humanized antibodies tested showed very good binding to the CD14 antigen. The affinity of humanized antibodies D6DD3D005, D6DD3D010, and D6DD3D015 are similar to the affinity of the mouse antibody D6DD3D001 (the original IgG1) prior to humanization, indicating that they have high binding affinity to CD14. Overall, the results indicate that antibodies of the present disclosure can bind with a CD14 antigen.

### Example 9. Expression and Affinity Testing of Antibodies Without PTM Sites

After the humanization of the antibodies, removal of post-translation modification (PTM) sites was performed as such sites can affect the stability of the antibody. Here, D6DD3D014 was selected for further investigations.

### 9.1. Experimental Materials

Experimental reagents and consumables included cell culture fluid, electroporation buffer, CHO cells, 1 mL electroporation cuvette, Protein A chip (Cytiva, 29127556), various sizes of shake flasks, and various sizes of pipettes. Main instruments and equipment included a shaker (Taicang Huamei), centrifuge (Baiyang), ultrapure water system (Millipore), constant temperature incubator (BLUE PARD), electroporator, water bath, Biacore 8K (Cytiva), and ELISA system (Molecular Devices).

The antigen used for the binding affinity measurements is B294801. This antigen is a human CD14 antigen that is histidine tagged. D6DD3D001 is a mouse antibody, and MPDDAD001-MPDDAD005 are humanized antibodies. The antibodies are recombinant, human (hFc Tag). Selected parameters of the experimental samples are shown in Table 9-1, with MW referring to molecular weight in kDa.

TABLE 9-1

| | Conc., | MW, | | |
|---|---|---|---|---|
| Name | mg/mL | kDa | Tag | Sample Type |
| D6DD3D001 | 1.2 | 146 | hFc | Original IgG1 antibody |
| MPDDAD001 | 1.29 | 144 | HFC | Antibody |
| MPDDAD002 | 1.44 | 144 | HFC | Antibody |
| MPDDAD003 | 1.62 | 144 | HFC | Antibody |
| MPDDAD004 | 1.33 | 144 | HFC | Antibody |
| MPDDAD005 | 1.16 | 144 | HFC | Antibody |
| B294801 | 17.24 | 36 | His | Antigen |

### 9.2. Experimental Method

### 9.2.1. Gene Construction

The heavy and light chain antibody genes of D6DD3D014 were cloned into a plasmid respectively. The amino acid sequences of the heavy and light chains are shown in Table 9-2 (antibody without PTM sites). Table 9-2 shows the amino acid sequences of heavy chain variable region (VH) and light chain variable region (VL) of various antibodies with PTM sites removed. The PTM removal sites (amino acid nos. 34, 35 of the VL) included: DS (MPDDAD001); SS (MPDDAD002); TS (MPDDAD003); NA (MPDDAD004); and QS (MPDDAD005).

TABLE 9-2
Antibody No. MPDDAD001

| | |
|---|---|
| VH | (SEQ ID NO: 13) |
| VL | DIQLTQSPSSLSASVGDRVTITCRASESVDSYVDSFLHWYQQKPGKA |
| | PKLLIYRASNLQSGVPSRFSGSGSRTDFTLTISSLQPEDVATYYCQQ |
| | SNEDPYTFGQGTKLEIK (SEQ ID NO: 151) |

Antibody No. MPDDAD002

| | |
|---|---|
| VH | (SEQ ID NO: 13) |
| VL | DIQLTQSPSSLSASVGDRVTITCRASESVDSYVSSFLHWYQQKPGKA |
| | PKLLIYRASNLQSGVPSRFSGSGSRTDFTLTISSLQPEDVATYYCQQ |
| | SNEDPYTFGQGTKLEIK (SEQ ID NO: 152) |

Antibody No. MPDDAD003

| | |
|---|---|
| VH | (SEQ ID NO: 13) |
| VL | DIQLTQSPSSLSASVGDRVTITCRASESVDSYVTSFLHWYQQKPGKA |
| | PKLLIYRASNLQSGVPSRFSGSGSRTDFTLTISSLQPEDVATYYCQQ |
| | SNEDPYTFGQGTKLEIK (SEQ ID NO: 153) |

Antibody No. MPDDAD004

| | |
|---|---|
| VH | (SEQ ID NO: 13) |
| VL | DIQLTQSPSSLSASVGDRVTITCRASESVDSYVNAFLHWYQQKPGKA |
| | PKLLIYRASNLQSGVPSRFSGSGSRTDFTLTISSLQPEDVATYYCQQ |
| | SNEDPYTFGQGTKLEIK (SEQ ID NO: 154) |

Antibody No. MPDDAD005

| | |
|---|---|
| VH | (SEQ ID NO: 13) |
| VL | DIQLTQSPSSLSASVGDRVTITCRASESVDSYVQSFLHWYQQKPGKA |
| | PKLLIYRASNLQSGVPSRFSGSGSRTDFTLTISSLQPEDVATYYCQQ |
| | SNEDPYTFGQGTKLEIK (SEQ ID NO: 155) |

### 9.2.2. Antibody Expression and Purification

Five modified antibodies with removed PTM sites were obtained by pairing light and heavy chains, yielding a total of five sets of plasmids after light and heavy chain pairing. About 145 million cells were centrifuged to remove the supernatant, and about 0.5 mL of electroporation solution was added to the cells. After thorough mixing, an appropriate amount of light and heavy chain paired plasmids (concentration 500 ng/μL) was added. After thoroughly mixing the above cell-plasmid suspension, 1 mL was taken and added to a 1 mL electroporation tube, and the electroporation tube was placed in the electroporation device for electroporation. After electroporation, the cells in the electroporation tube were divided into pre-prepared flasks containing 20 mL of culture medium and incubated for 40 minutes. After incubation, the flasks were placed in a 37° C., 270 rpm, 8% CO2 incubator. After 4 days of culture, the supernatant was collected by centrifugation (8000 rpm, 5 min) and purified using a Protein A affinity chromatography column.

### 9.3.3. Antibody Affinity Test

The Biacore 8K platform was used to test the kinetic and affinity properties of the antibody, operating according to system instructions and indications. The experimental parameters used are shown in Table 9-3.

TABLE 9-3
Detection parameters

| Parameter | Selected details |
|---|---|
| Capture ligand | 1 μg/mL antibody in Running Buffer, |
| | 10 μL/min |
| Running buffer | HBS-EP + Buffer (Cytiva) |
| Association and | 30 μL/min, Association 100 s, |
| dissociation flow rate | Dissociation 200 s |
| Regeneration buffer | 10 mM pH 1.5 Glycine-HCl (Gly-HCl) |
| Regeneration flow rate | 30 μL/min, 30 s |

### 9.3. Experimental Results

Results of Antibody Expression and Purification. Five antibodies with removed PTM sites (coded MPDDAD001-MPDDAD005) and the murine antibody (coded D6DD3D001) were tested using SEC and SDS-PAGE. By SDS-Page, the antibodies had a purity of >95%. Table 9-4 shows selected results such as concentration (Conc.) and purity. Purity by SEC was detected at an absorbance of 214 nm and at an absorbance of 280 nm. Purity values in Table 9-4 are in units of percent, %.

TABLE 9-4

| | | Purity | Purity |
|---|---|---|---|
| | Conc., | (SEC, | (SEC, |
| Antibody No. (Name) | mg/mL | 214 nm) | 280 nm) |
| D6DD3D001 (HU1127-mVH/mVL) | 1.2 | 98.99 | 98.686 |
| MPDDAD001 (HU1127-hVH3/hVL3-1) | 1.29 | 98.19 | 98.44 |
| MPDDAD002 (HU1127-hVH3/hVL3-2) | 1.44 | 98.82 | 98.99 |
| MPDDAD003 (HU1127-hVH3/hVL3-3) | 1.62 | 98.85 | 98.95 |
| MPDDAD004 (HU1127-hVH3/hVL3-4) | 1.33 | 97.04 | 97.2 |
| MPDDAD005 (HU1127-hVH3/hVL3-5) | 1.16 | 99.18 | 99.24 |

Results of Affinity Detection. The affinity of the six antibodies was measured at saturation concentration. For the experiments, the analyte solution comprises the B294801 antigen with an analyte concentration of 0.1953125 nM, 0.390625 nM, 0.78125 nM, 1.5625 nM, 3.125 nM, 6.25 nM, 12.5 nM, and 25 nM. Selected results are shown in Table 9-4. Change factor is described above.

TABLE 9-4
Affinity detection results at full concentration

| | Capture | | | | |
|---|---|---|---|---|---|
| | conc., | 1:1 binding | K_{off}, | KD, | Change |
| Ligand | μg/mL | kₒₙ, 1/Ms | 1/s | M | Factor |
| D6DD3D001 | 1 | 1.25E+06 | 1.08E−03 | 8.64E−10 | 1.00 |
| MPDDAD001 | 1 | 8.47E+05 | 6.33E−03 | 7.48E−09 | 8.66 |
| MPDDAD002 | 1 | 1.29E+06 | 3.62E−03 | 2.80E−09 | 3.24 |
| MPDDAD003 | 1 | 1.29E+06 | 2.60E−03 | 2.02E−09 | 2.34 |
| MPDDAD004 | 1 | 7.75E+05 | 4.60E−03 | 5.93E−09 | 6.86 |
| MPDDAD005 | 1 | 1.11E+06 | 5.44E−03 | 4.88E−09 | 5.65 |

The results indicate that the affinity of the antibody MPDDAD003, with removed PTM sites, is approximately equivalent to the affinity of the humanized antibody D6DD3D014, and it is within three times less than the affinity of the murine antibody D6DD3D001 (original IgG1). In this experiment, the high-risk PTM site (NS, in the light chain CDR1 of the humanized antibody D6DD3D014) was successfully removed.

The humanization design of the murine antibody adopted the CDR grafting method. Homologous proteins of the murine antibody V region were database searched, and 5 human heavy chain germline framework sequences and 3 human light chain germline framework sequences were selected as templates for humanization design. The CDR region of the murine antibody was transplanted into the human germline framework area, and several back mutation sites were then selected on the generated humanized sequences. After pairing the humanized heavy and light chains, 15 humanized antibody sequences (D6DD3D002-D6DD3D0016) were obtained.

After gene cloning and cell transfection, 15 humanized Atibuclimab antibodies and the murine antibody were expressed. After affinity detection with the Biacore platform, it was found that the affinity of most humanized antibodies decreased to varying degrees, and some humanized antibodies showed affinity that were approximately equivalent to the murine antibody. Among them, the affinities of the humanized antibodies D6DD3D005, D6DD3D010, and D6DD3D015 were approximately equivalent to the affinity of the antibody D6DD3D001 before humanization.

Five humanized antibodies with removed PTM sites were expressed after gene cloning and cell transfection. After affinity detection with the Biacore platform, it was found that the affinity of some humanized antibodies with removed PTM sites decreased to varying degrees, and some showed affinity approximately equivalent to the humanized antibodies. Among them, the affinity of the antibody with removed PTM sites, MPDDAD003, was approximately equivalent to the affinity of the humanized antibody D6DD3D014, and it was within three times less than the affinity of the murine antibody D6DD3D001.

### EMBODIMENTS LISTING

The present disclosure provides, among others, the following aspects, each of which can be considered as optionally including any alternate aspects:

Clause A1. An antibody specifically binding to CD14, or an antigen binding fragment thereof, comprising: a heavy chain variable region (VH) comprising HCDRs 1-3 having amino acid sequences as set forth in SEQ ID NOs: 131-133, respectively; and a light chain variable region (VL) comprising LCDRs 1-3 having amino acid sequences set forth in SEQ ID NOs: 134-136, respectively.

Clause A2. The antibody or antigen binding fragment thereof according to Clause A1, wherein: the VH comprises an amino acid sequence as set forth in one or more of SEQ ID NOs: 1, 17, 21, 37, 41, 57, or 61, or an amino acid sequence having at least 80% sequence identity to one or more of SEQ ID NOs: 1, 17, 21, 37, 41, 57, or 61; and the VL comprises an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63.

Clause A3. The antibody or antigen binding fragment thereof according to any one of Clauses A1 or A2, wherein the antibody is of an isotype selected from the group consisting of IgG, IgA, IgM, IgE, and IgD.

Clause A4. The antibody or the antigen binding fragment thereof according to any one of Clauses A1-A3, wherein the antibody is of a subtype selected from the group consisting of IgG1, IgG2, and IgG3.

Clause A5. The antibody or the antigen binding fragment thereof according to any one of Clauses A1-A4, wherein the antigen binding fragment is selected from the group consisting of Fab, Fab′, F(ab′)2, Fv, scFv, and ds-scFv.

Clause A6. The antibody or the antigen binding fragment thereof according to any one of Clauses A1-A5, wherein the antibody is a monoclonal antibody.

Clause A7. The antibody or the antigen binding fragment thereof according to any one of Clauses A1-A6, wherein the antibody or the antigen binding fragment thereof comprises: a heavy chain comprising an amino acid sequence as set forth in one or more of SEQ ID NOs: 2, 18, 22, 38, 42, 58, or 62, or an amino acid sequence having at least 80% sequence identity to one or more of SEQ ID NOs: 2, 18, 22, 38, 42, 58, or 62; and a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

Clause A8. A nucleic acid molecule comprising a nucleotide sequence encoding the antibody or the antigen binding fragment thereof according to any one of Clauses A1-A7.

Clause A9. A pharmaceutical composition comprising the antibody or the antigen binding fragment thereof according to any one of Clauses A1-A8.

Clause B1. An antibody specifically binding to CD14, or an antigen binding fragment thereof, comprising: a heavy chain variable region (VH) comprising HCDRs 1-3 having amino acid sequences as set forth in SEQ ID NOs: 158, 132, and 133, respectively; and a light chain variable region (VL) comprising LCDRs 1-3 having amino acid sequences set forth in SEQ ID NOs: 134-136, respectively.

Clause B2. The antibody or antigen binding fragment thereof according to Clause B1, wherein: the VH comprises an amino acid sequence as set forth in one or more of SEQ ID NOs: 5, 9, 13, 25, 29, 33, 45, 49, or 53, or an amino acid sequence having at least 80% sequence identity to one or more of SEQ ID NOs: 5, 9, 13, 25, 29, 33, 45, 49, or 53; the VL comprises an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63.

Clause B3. The antibody or antigen binding fragment thereof according to Clause B1 or Clause B2, wherein: the antibody is of an isotype selected from the group consisting of IgG, IgA, IgM, IgE, and IgD; the antibody is of a subtype selected from the group consisting of IgG1, IgG2, and IgG3; and/or combinations thereof.

Clause B4. The antibody or the antigen binding fragment thereof according to any one of Clauses B1-B3, wherein the antigen binding fragment is selected from the group consisting of Fab, Fab′, F(ab′)2, Fv, scFv, and ds-scFv

Clause B5. The antibody or the antigen binding fragment thereof according to any one of Clauses B1-B4, wherein the antibody is a monoclonal antibody.

Clause B6. The antibody or the antigen binding fragment thereof according to any one of Clauses B1-B5, wherein the antibody or the antigen binding fragment thereof comprises: a heavy chain comprising an amino acid sequence as set forth in one or more of SEQ ID NOs: 6, 10, 14, 26, 30, 34, 46, 50, or 54, or an amino acid sequence having at least 80% sequence identity to one or more of SEQ ID NOs: 6, 10, 14, 26, 30, 34, 46, 50, or 54; and a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

Clause B7. A nucleic acid molecule comprising a nucleotide sequence encoding the antibody or the antigen binding fragment thereof according to any one of Clauses B1-B6.

Clause B8. A pharmaceutical composition comprising the antibody or the antigen binding fragment thereof according any one of Clauses B1-B7.

Clause C1. A pharmaceutical composition, comprising: (i) an antibody specifically binding to CD14 or an antigen binding fragment thereof, comprising: a heavy chain variable region (VH) comprising HCDRs 1-3 having amino acid sequences as set forth in SEQ ID NOs: 131-133, respectively; and a light chain variable region (VL) comprising LCDRs 1-3 having amino acid sequences set forth in SEQ ID NOs: 134-136, respectively; and (ii) a pharmaceutically acceptable carrier or excipient.

Clause C2. The pharmaceutical composition according to Clause C1, further comprising a second therapeutic agent.

Clause C3. The pharmaceutical composition according to Clause C2, wherein the second therapeutic agent comprises a small molecule cytotoxic agent.

Clause C4. The pharmaceutical composition according to Clause C3, wherein the small molecule cytotoxic agent comprises cisplatin.

Clause C5. The pharmaceutical composition according to any one of Clauses C1-C4, wherein: the VH comprises an amino acid sequence as set forth in one or more of SEQ ID NOs: 1, 17, 21, 37, 41, 57, or 61, or an amino acid sequence having at least 80% sequence identity to one or more of SEQ ID NOs: 1, 17, 21, 37, 41, 57, or 61; and the VL comprises an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63.

Clause C6. The pharmaceutical composition according to any one of Clauses C1-C5, wherein the antibody or the antigen binding fragment thereof is of an isotype selected from the group consisting of IgG, IgA, IgM, IgE, and IgD.

Clause C7. The pharmaceutical composition according to any one of Clauses C1-C6, wherein the antibody or the antigen binding fragment thereof is of a subtype selected from the group consisting of IgG1, IgG2, IgG3, and IgG4.

Clause C8. The pharmaceutical composition according to any one of Clauses C1-C7, wherein the antibody or the antigen binding fragment thereof is selected from the group consisting of Fab, Fab′, F(ab′)2, Fv, scFv, and ds-scFv.

Clause C9. The pharmaceutical composition according to any one of Clauses C1-C8, wherein the antibody or the antigen binding fragment thereof is a monoclonal antibody.

Clause D1. A pharmaceutical composition, comprising: (i) an antibody specifically binding to CD14 or an antigen binding fragment thereof, comprising: a heavy chain variable region (VH) comprising HCDRs 1-3 having amino acid sequences as set forth in SEQ ID NOs: 158, 132, and 133, respectively; and a light chain variable region (VL) comprising LCDRs 1-3 having amino acid sequences set forth in SEQ ID NOs: 134-136, respectively; and (ii) a pharmaceutically acceptable carrier or excipient.

Clause D2. The pharmaceutical composition according to Clause D1, further comprising a second therapeutic agent.

Clause D3. The pharmaceutical composition according to Clause D2, wherein the second therapeutic agent comprises a small molecule cytotoxic agent.

Clause D4. The pharmaceutical composition according to Clause D3, wherein the small molecule cytotoxic agent comprises cisplatin.

Clause D5. The pharmaceutical composition according to any one of Clauses D1-D4, wherein: the VH comprises an amino acid sequence as set forth in one or more of SEQ ID NOs: 5, 9, 13, 25, 29, 33, 45, 49, or 53, or an amino acid sequence having at least 80% sequence identity to one or more of SEQ ID NOs: 5, 9, 13, 25, 29, 33, 45, 49, or 53; and the VL comprises an amino acid sequence as set forth in one or more of SEQ ID NOS: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63.

Clause D6. The pharmaceutical composition according to any one of Clauses D1-D5, wherein the antibody or the antigen binding fragment thereof is of an isotype selected from the group consisting of IgG, IgA, IgM, IgE, and IgD.

Clause D7. The pharmaceutical composition according to any one of Clauses D1-D6, wherein the antibody or the antigen binding fragment thereof is of a subtype selected from the group consisting of IgG1, IgG2, IgG3, and IgG4.

Clause D8. The pharmaceutical composition according to any one of Clauses D1-D7, wherein the antibody or the antigen binding fragment thereof is selected from the group consisting of Fab, Fab′, F(ab′)2, Fv, scFv, and ds-scFv.

Clause D9. The pharmaceutical composition according to any one of Clauses D1-D8, wherein the antibody or the antigen binding fragment thereof is a monoclonal antibody.

Clause E1. A method of treating bladder cancer or recurrent bladder cancer in a patient, comprising: administering to a patient a therapy comprising: an antibody specifically binding to CD14 or an antigen binding fragment thereof, comprising: a heavy chain variable region (VH) comprising HCDRs 1-3 having amino acid sequences as set forth in SEQ ID NOs: 131-133, respectively; and a light chain variable region (VL) comprising LCDRs 1-3 having amino acid sequences set forth in SEQ ID NOs: 134-136, respectively; wherein the therapy is administered over a 28-day cycle.

Clause E2. The method according to Clause E1, wherein the therapy further comprises a small molecule cytotoxic agent.

Clause E3. The method according to Clause E1 or E2, wherein the antibody or the antigen binding fragment thereof comprises: a heavy chain comprising an amino acid sequence as set forth in one or more of SEQ ID NOs: 2, 18, 22, 38, 42, 58, or 62, or an amino acid sequence having at least 80% sequence identity to one or more of SEQ ID NOS: 2, 18, 22, 38, 42, 58, or 62; and a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

Clause F1. A method of treating bladder cancer or recurrent bladder cancer in a patient, comprising: administering to a patient a therapy comprising: an antibody specifically binding to CD14 or an antigen binding fragment thereof, comprising: a heavy chain variable region (VH) comprising HCDRs 1-3 having amino acid sequences as set forth in SEQ ID NOs: 158, 132, and 133, respectively; and a light chain variable region (VL) comprising LCDRs 1-3 having amino acid sequences set forth in SEQ ID NOs: 134-136, respectively; wherein the therapy is administered over a 28-day cycle.

Clause F2. The method according to Clause F1, wherein the therapy further comprises a small molecule cytotoxic agent.

Clause F3. The method according to Clause F1 or Clause F2, wherein the antibody or the antigen binding fragment thereof comprises: a heavy chain comprising an amino acid sequence as set forth in one or more of SEQ ID NOs: 6, 10, 14, 26, 30, 34, 46, 50, or 54, or an amino acid sequence having at least 80% sequence identity to one or more of SEQ ID NOs: 6, 10, 14, 26, 30, 34, 46, 50, or 54; and a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

Embodiments described herein generally relate to antibodies against CD14, and uses of such antibodies, in particular their use in the treatment of cancer in a patient. Overall, antibodies of the present disclosure can be utilized to specifically and potently inhibit growth of tumor cells expressing CD14, suggesting their potential use for treating CD14 positive cancers.

All documents described herein are incorporated by reference herein, including any priority documents and/or testing procedures to the extent they are not inconsistent with this text. As is apparent from the foregoing general description and the specific embodiments, while forms of the embodiments have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element, a group of elements, or a method is preceded with the transitional phrase “comprising,” it is understood that we also contemplate the same composition, method, or group of elements with transitional phrases “consisting essentially of,” “consisting of,” “selected from the group of consisting of,” or “Is” preceding the recitation of the composition, element, elements, or method, and vice versa, such as the terms “comprising,” “consisting essentially of,” “consisting of” also include the product of the combinations of elements listed after the term.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter described herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly described herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, embodiments comprising “an antibody” include embodiments comprising one, two, or more antibodies, unless specified to the contrary or the context clearly indicates only one antibody is included.

While the foregoing is directed to embodiments of the present disclosure, other and further embodiments of the disclosure may be devised without departing from the basic scope thereof, and the scope thereof is determined by the claims that follow.

What is claimed is:

1. An antibody specifically binding to CD14, or an antigen binding fragment thereof, comprising:
a heavy chain variable region (VH) comprising HCDRs 1-3 having amino acid sequences as set forth in SEQ ID NOs: 131-133, respectively; and
a light chain variable region (VL) comprising LCDRs 1-3 having amino acid sequences set forth in SEQ ID NOs: 134-136, respectively.

2. The antibody or antigen binding fragment thereof according to claim 1, wherein:
the VH comprises an amino acid sequence as set forth in one or more of SEQ ID NOs: 1, 17, 21, 37, 41, 57, or 61, or an amino acid sequence having at least 80% sequence identity to one or more of SEQ ID NOs: 1, 17, 21, 37, 41, 57, or 61; and
the VL comprises an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63.

3. The antibody or antigen binding fragment thereof according to claim 1, wherein:
the antibody is of an isotype selected from the group consisting of IgG, IgA, IgM, IgE, and IgD.

4. The antibody or the antigen binding fragment thereof according to claim 1, wherein the antibody is of a subtype selected from the group consisting of IgG1, IgG2, and IgG3.

5. The antibody or the antigen binding fragment thereof according to claim 1, wherein the antigen binding fragment is selected from the group consisting of Fab, Fab′, F(ab′)2, Fv, scFv, and ds-scFv.

6. The antibody or the antigen binding fragment thereof according to claim 1, wherein the antibody is a monoclonal antibody.

7. The antibody or the antigen binding fragment thereof according to claim 1, wherein the antibody or the antigen binding fragment thereof comprises:
a heavy chain comprising an amino acid sequence as set forth in one or more of SEQ ID NOs: 2, 18, 22, 38, 42, 58, or 62, or an amino acid sequence having at least 80% sequence identity to one or more of SEQ ID NOs: 2, 18, 22, 38, 42, 58, or 62; and
a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

8. A nucleic acid molecule comprising a nucleotide sequence encoding the antibody or the antigen binding fragment thereof according to claim 1.

9. A pharmaceutical composition comprising the antibody or the antigen binding fragment thereof according to claim 1.

10. An antibody specifically binding to CD14, or an antigen binding fragment thereof, comprising:
a heavy chain variable region (VH) comprising HCDRs 1-3 having amino acid sequences as set forth in SEQ ID NOs: 158, 132, and 133, respectively; and
a light chain variable region (VL) comprising LCDRs 1-3 having amino acid sequences set forth in SEQ ID NOs: 134-136, respectively.

11. The antibody or antigen binding fragment thereof according to claim 10, wherein:
the VH comprises an amino acid sequence as set forth in one or more of SEQ ID NOs: 5, 9, 13, 25, 29, 33, 45, 49, or 53, or an amino acid sequence having at least 80% sequence identity to one or more of SEQ ID NOs: 5, 9, 13, 25, 29, 33, 45, 49, or 53; and
the VL comprises an amino acid sequence as set forth in one or more of SEQ ID NOs: 3, 7, 11, 15, 19, 23, 27, 31, 35, 39, 43, 47, 51, 55, 59, or 63, or an amino acid sequence having at least 80% sequence identity to one or more of SEQ ID NOs: 3, 7, 11, 15, 19, 23, 27, 31, 35, 39, 43, 47, 51, 55, 59, or 63.

12. The antibody or antigen binding fragment thereof according to claim 10, wherein:
the antibody is of an isotype selected from the group consisting of IgG, IgA, IgM, IgE, and IgD;
wherein the antibody is of a subtype selected from the group consisting of IgG1, IgG2, and IgG3;
or combinations thereof.

13. The antibody or the antigen binding fragment thereof according to claim 10, wherein the antigen binding fragment is selected from the group consisting of Fab, Fab′, F(ab′)2, Fv, scFv, and ds-scFv.

14. The antibody or the antigen binding fragment thereof according to claim 10, wherein the antibody is a monoclonal antibody.

15. The antibody or the antigen binding fragment thereof according to claim 10, wherein the antibody or the antigen binding fragment thereof comprises:
a heavy chain comprising an amino acid sequence as set forth in one or more of SEQ ID NOs: 6, 10, 14, 26, 30, 34, 46, 50, or 54, or an amino acid sequence having at least 80% sequence identity to one or more of SEQ ID NOs: 6, 10, 14, 26, 30, 34, 46, 50, or 54; and
a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.

16. A nucleic acid molecule comprising a nucleotide sequence encoding the antibody or the antigen binding fragment thereof according to claim 10.

17. A pharmaceutical composition comprising the antibody or the antigen binding fragment thereof according to claim 10.

18. A method of treating bladder cancer or recurrent bladder cancer in a patient, comprising:
administering to a patient a therapy comprising:
an antibody specifically binding to CD14 or an antigen binding fragment thereof, comprising:
a heavy chain variable region (VH) comprising HCDRs 1-3 having amino acid sequences as set forth in SEQ ID NOs: 131-133, respectively; and
a light chain variable region (VL) comprising LCDRs 1-3 having amino acid sequences set forth in SEQ ID NOs: 134-136, respectively;
wherein the therapy is administered over a 28-day cycle.

19. The method of claim 18, wherein the therapy further comprises a small molecule cytotoxic agent.

20. The method of claim 18, wherein the antibody or the antigen binding fragment thereof comprises:
a heavy chain comprising an amino acid sequence as set forth in one or more of SEQ ID NOs: 2, 6, 10, 14, 18, 22, 26, 30, 34, 38, 42, 46, 50, 54, 58, or 62, or an amino acid sequence having at least 80% sequence identity to one or more of SEQ ID NOs: 2, 6, 10, 14, 18, 22, 26, 30, 34, 38, 42, 46, 50, 54, 58, or 62; and
a light chain comprising an amino acid sequence as set forth in one or more of SEQ ID NOs: 4, 8, 12, 16, 20, 24, 28, 32, 36, 40, 44, 48, 52, 56, 60, or 64, or an amino acid sequence having at least 80% sequence identity to one or more of SEQ ID NOs: 4, 8, 12, 16, 20, 24, 28, 32, 36, 40, 44, 48, 52, 56, 60, or 64.
